(19) United States
(12) Patent Application Publication
(10) Pub. No.: US 2021/0255189 A1
(43) Pub. Date: Aug. 19, 2021

(54) BIOMARKER PANEL FOR OVARIAN CANCER

(71) Applicant: Olink Proteomics AB, Uppsala (SE)

(72) Inventors: Ulf GYLLENSTEN, Uppsala (SE); Stefan ENROTH, Uppsala (SE); Karin SUNDFELDT, Torslanda (SE); Martin LUNDBERG, Uppsala (SE); John BROBERG, Uppsala (SE)

(21) Appl. No.: 17/252,102

(22) PCT Filed: Jun. 14, 2019
(86) PCT No.: PCT/EP19/65730

§ 371 Date: Dec. 14, 2020

(30) Foreign Application Priority Data
Jun. 15, 2018 (GB) 1809854.1
Jun. 15, 2018 (GB) 1809858.2
Jun. 15, 2018 (GB) 1809862.4
Jun. 15, 2018 (GB) 1809868.1
Jun. 15, 2018 (GB) 1809869.9
Jun. 15, 2018 (GB) 1809872.3
Jun. 15, 2018 (GB) 1809873.1
Jun. 15, 2018 (GB) 1809875.6
Jun. 15, 2018 (GB) 1809876.4
Jun. 15, 2018 (GB) 1809878.0
Jun. 15, 2018 (GB) 1809881.4
Jun. 15, 2018 (GB) 1809882.2
Jun. 15, 2018 (GB) 1809885.5

(51) Int. Cl. G01N 33/574

(52) U.S. Cl. CPC G01N 33/57449; G01N 2800/52; G01N 2800/50

(57) ABSTRACT

The present invention relates to the identification of novel panels and combinations of biomarkers for ovarian cancer. An in vitro method is provided for detecting, predicting or monitoring ovarian cancer in a subject, wherein said method comprises determining in a sample from said subject the levels of the biomarkers in a panel comprising: TACSTD2, PROK1, MSMB, MUC-16, WFDC2, FR-alpha, and KRT19. More broadly, biomarkers may be selected and used from this list in variations combinations with each other and with other biomarkers. Also provided are sets of reagents for use in such methods.

The present invention relates to the field of biomarkers for the detection of cancer in a subject. More particularly, the present invention provides biomarkers and biomarker panels which may be used in methods of identifying a subject having ovarian cancer, and reagents therefor. Particularly, sets of reagents for the detection of panels of such biomarkers are provided.

Ovarian cancer is the seventh most common cancer in women worldwide (18 most common cancer overall), with 238,719 new cases diagnosed in 2012, representing 3.4% of all cancer. A major problem is the difficulty to identify women with early-stage ovarian cancer; ovarian cancer often has no symptoms at the early stages, so the disease is generally advanced when it is diagnosed. Approximately 70 to 75% of newly-diagnosed cases have late stage disease, and the five year survival rate is only 25 to 30%. Ovarian diseases require surgical sampling for proper diagnosis with potential effects on reproductive capacity. There is therefore a strong need to develop improved pre-operative diagnostic methods for women with pelvic symptoms.

Mucin-16 (MUC-16 or Cancer antigen 125, CA-125) was introduced as a biomarker for ovarian cancer in 1983 and is currently the most important single biomarker for epithelial ovarian cancer. Mucin-16 alone however, has low sensitivity for early stage cancer (50-62%) at a specificity of 94-98.5%. Combinations of mucin-16 and other biomarkers, including the human epididymal protein 4 (HE4), increases the sensitivity to 75% at similar specificity (90-95%). The low sensitivity for detection of early stage ovarian cancer prohibits population screening using the current biomarkers. A recent study in the UK suggests that the current tests are approaching sufficient accuracy from a health-economic stand-point to justify screening. However, tests with low specificity have a high false positive rate, and results in that many women will be unnecessarily examined causing anxiety and additional cost for the health-care system.

Reliable diagnostic tests to differentiate between benign and malignant lesions are also lacking. MUC-16 and HE4 are Federal Drug Administration (FDA) approved biomarkers for ovarian cancer, and are sometimes used in pre-operative diagnostics. The presently available biomarkers are mainly used to improve diagnosis of women that experience symptoms and when the transvaginal ultrasound (TVU) indicate adnexal ovarian mass. The tests then triage patients in need of surgery and those with benign conditions. Even in this context, identification of clinically useful biomarkers based on single or combination of proteins is challenging. The risk of malignancy index (RMI), a score based on CA125 plasma levels, menopause status and transvaginal ultrasound (TVU) criterion, was developed to triage women to specialized cancer units. A few algorithms based on biomarker panels assayed in blood have been FDA approved. The risk of ovarian malignancy algorithm (ROMA) uses CA125, HE4 and menopausal status to assign women with adnexal ovarian mass into a high-risk and low-risk group for ovarian malignancy (Moore, R. G., et al. 2011. Obstet Gynecol, 2011. 118(2 Pt 1): p. 280-8). The multivariate index assay (MIA) Overa® is based on CA125-II, HE4, apolipoprotein A-1, follicle stimulating hormone and transferrin, and was FDA approved in 2016. Overa® has a specificity of 0.91 and a sensitivity of 0.69 to distinguish between benign tumors and ovarian cancer (Coleman, R. L., et al. 2016. Am J Obstet Gynecol, 2016. 215(1): p. 82 e1-82 ell). Additional plasma protein markers have also been described (Boylan, K. L. M., et al. 2017. Clin Proteomics, 2017. 14: p. 34).

Longitudinal testing has been proposed as an alternative to the use of a single diagnostic test for screening in a healthy population. The Risk of Ovarian Cancer Algorithm (ROCA) is based on longitudinal (annual) measurements of CA125 in a screening population in order to identify women with high-risk scores and refer these to specialized units for TVU examination. Use of ROCA followed by TVU has been shown to result in a doubling of the number of women with OCs detected as compared to the use of a fixed cutoff for CA125 only. The United Kingdom Collaborative Trial of Ovarian Cancer Screening (UKCTOCS) reported a reduction in ovarian cancer deaths using annual multimodal screening, but only when prevalent cases were excluded.

Despite the recent advances in the testing for ovarian cancer, there are no generally applicable tests which can be used in general population screening in order to identify women who are suffering from ovarian cancer, with sufficient sensitivity and specificity. Moreover, tests which are currently available for detecting ovarian cancer are not capable of differentiating between ovarian cancer (i.e. malignant cancer) and benign ovarian tumours with sufficient sensitivity and/or specificity. Together, this means that current tests for ovarian cancer provide unacceptably high rates of both false negative test results (i.e. indicating that a woman does not have ovarian cancer when in fact they do), and false positive test results (i.e. indicating that a woman has ovarian cancer, when in fact she may be healthy or have only a benign, non-cancerous, tumour which does not require treatment). False negative results may result in a delay in providing treatment to a woman suffering from ovarian cancer, whereas false positive results may result in unnecessary invasive and costly further testing being undertaken.

There is therefore a need to identify additional diagnostic biomarkers which are associated with ovarian cancer (in particular, malignant cancer), in order to allow ovarian cancer in a subject to be detected, predicted or monitored with greater sensitivity and specificity.

The present invention provides new insights into biomarkers which are associated with ovarian cancer. Not only does the present invention identify new biomarkers for detecting, predicting or monitoring ovarian cancer in a subject which had not previously been associated with ovarian cancer, it also provides panels of biomarkers which can be used to detect, predict or monitor ovarian cancer with greater sensitivity and specificity, and sets of reagents for the same. In particular, we have identified that panels based on known biomarkers may be improved by the addition of additional biomarkers, and more particularly that the use of MUC-16 as a biomarker may be improved by the addition of additional biomarkers.

Recent developments of high-throughput technologies for detection and quantification of proteins has made it possible to study thousands of biomarker candidates in a single sample and to a low cost. In the work leading to the present invention, plasma samples from 169 healthy patients, patients with benign tumours, and patients with various stages of ovarian cancer were screened for 981 unique proteins by the proximity extension assay (PEA) using eleven Olink Proteomics multiplex panels (Oncology II, Cardiovascular II, Cardiovascular III, Neurology, Inflammation, Cardiometabolic, Cell Regulation, Development, Immune Response, Metabolism and Organ Damage panels) in a discovery step, in order to identify biomarkers which might be indicative of ovarian cancer in a subject. Surprisingly, a number of biomarkers which were not previously known to be specifically associated with ovarian cancer were identified in this analysis (in addition to known markers for ovarian cancer such as WFDC2/HE4 and CA125/MUC-16) as being beneficial in the diagnosis of ovarian cancer. Subsequent testing of the identified biomarkers on a replication cohort demonstrated their utility in diagnosing ovarian cancer. Specifically, this analysis identified IL10, KLK10, KRT19, MUC-16, PTK7, SEZ6L, SPINT1, TACSTD2, CD83, CD99L2, CLEC4D, CLEC6A, CLMP, CPE, FR-alpha, ICOSLG, IRF9, LAMP3, MSMB, PROK1, SIT1, SKAP1, SPINK5, TANK and WFDC2 as being of use in the detection of ovarian cancer. A full list of these proteins and their respective UNIPROT numbers is provided in Table 1.

TABLE 1
list of biomarkers and UNIPROT numbers.

| Protein name | UNIPROT number |
|---|---|
| IL10 | P22301 |
| KLK10 | O43240 |
| KRT19 | P08727 |
| MUC-16 | Q8WXI7 |
| PTK7 | Q13308 |
| SEZ6L2 | Q6UXD5 |
| SPINT1 | O43278 |
| TACSTD2 | P09758 |
| CD83 | Q01151 |
| CD99L2 | Q8TCZ2 |
| CLEC4D | Q8WXI8 |
| CLEC6A | Q6EIG7 |
| CLMP | Q9H6B4 |
| CPE | P16870 |
| FR-alpha | P15328 |
| ICOSLG | O75144 |
| I RF9 | Q00978 |
| LAMP3 | Q9UQV4 |
| MSMB | P08118 |
| PROK1 | P58294 |
| SIT1 | Q9Y3P8 |
| SKAP1 | Q86WV1 |
| SPINK5 | Q9NQ38 |
| TANK | Q92844 |
| WFDC2 | Q14508 |
| CDH3 | P22223 |

Further details on the protocols used and the discovery and replication experiments are provided in the Examples below.

The present invention therefore provides methods for detecting, predicting or monitoring ovarian cancer in a subject, comprising determining the level of a particular biomarker or of panels of particular biomarkers in a sample from a subject. The use of individual biomarkers, as well as panels of biomarkers in such methods, is provided. Sets of reagents for detecting such panels of biomarkers are also provided.

Described herein are in vitro methods of detecting, predicting or monitoring ovarian cancer in a subject, comprising determining in a sample from said subject the level of a biomarker selected from the list consisting of IL10, KLK10, KRT19, MUC-16, PTK7, SEZ6L, SPINT1, TACSTD2, CD83, CD99L2, CLEC4D, CLEC6A, CLMP, CPE, FR-alpha, ICOSLG, IRF9, LAMP3, MSMB, PROK1, SIT1, SKAP1, SPINK5, TANK and WFDC2. CDH3 may further be included in this list (as can be seen from Example 3 below).

The use of IL10, KLK10, KRT19, MUC-16, PTK7, SEZ6L, SPINT1, TACSTD2, CD83, CD99L2, CLEC4D, CLEC6A, CLMP, CPE, FR-alpha, ICOSLG, IRF9, LAMP3, MSMB, PROK1, SIT1, SKAP1, SPINK5, TANK or WFDC2, particularly TACSTD2, SPINT1, ICOSLG, PTK7, SEZ6L2, SPINK5, CLEC6A, CLEC4D, SKAP1, CD83, PROK1, MSMB or CPE as a biomarker for ovarian cancer are also described. CDH3 may further be included in this list.

As described in greater detail below, the level of biomarkers that is determined may be compared with the level in a control group (e.g. comprising healthy subjects who do not have ovarian cancer) or to reference level (s). A biomarker profile may be obtained, which may be compared to a reference or control biomarker profile. Divergence between the determined level of biomarkers in the subject sample and the control group or a reference level, or similarity between the level of biomarkers in the subject sample and a known ovarian cancer profile may allow ovarian cancer to be detected, predicted or monitored. Any of the biomarkers identified in the work leading to the present invention may be used as a biomarker for detecting, predicting or monitoring ovarian cancer in a subject.

Various aspects of the present disclosure and invention are presented below, based on the various different biomarkers used individually or in different combinations. In particular, we include in further aspects below, particular combinations, or panels, of biomarkers which we have now found to be particularly suitable for detecting, predicting or monitoring ovarian cancer.

In a first aspect, the present invention provides a method, particularly an in vitro method, of detecting, predicting or monitoring ovarian cancer in a subject, comprising determining in a sample from said subject the level of a biomarker selected from the list consisting of TACSTD2, SPINT1, ICOSLG, PTK7, SEZ6L2, SPINK5, CLEC6A, CLEC4D, SKAP1, CD83, PROK1, MSMB and CPE.

In another aspect, the use of TACSTD2, SPINT1, ICOSLG, PTK7, SEZ6L2, SPINK5, CLEC6A, CLEC4D, SKAP1, CD83, PROK1, MSMB or CPE as a biomarker for ovarian cancer is provided.

In particular, the biomarkers may have utility as biomarkers of ovarian cancer when detected other than directly within samples of ovarian tissue, that is other than in an in situ context in a sample of ovarian tissue (i.e. not in ovarian tissue as such). For example, as discussed further below, the biomarker(s) may be detected in tissue or cell lysates, or in fine needle biopsies, including of ovarian tissue. Alternatively, the samples may be samples which are not of ovarian tissue (i.e. the biomarker may be detected in a non-ovarian sample, or in a tissue or body fluid sample other than ovarian tissue). In other words, the level of the biomarker in a non-ovarian tissue sample (which refers to samples both of healthy or normal ovarian tissue, or diseased, e.g. ovarian cancer, tissue) may be indicative of ovarian cancer. Thus, in certain embodiments of the methods and uses of this and other aspects of the invention, as detailed below, the level of the biomarker(s) is not determined in situ in an ovarian tissue sample. Alternatively expressed, the level of the biomarkers is determined other than in situ in an ovarian tissue sample. In other embodiments of the methods and uses of this and other aspects of the invention, as detailed below, the level of the biomarker(s) may be determined in a sample of non-ovarian tissue or body fluid, and particularly in a blood or blood-derived sample.

It was found that determining the levels of two or more of the biomarkers which were identified in the work leading to the present invention may provide more reliable tests for detecting, predicting or monitoring ovarian cancer in a subject.

Particular biomarkers from within the biomarkers identified herein have been found to be surprisingly effective biomarkers for diagnosing ovarian cancer, and were found to account for a greater proportion of the variability between a control group and a patient group than other biomarkers (and thus may be more promising biomarkers for diagnosing ovarian cancer), and in particular were found to be effective at discriminating between a control subject and a subject with ovarian cancer when used in pairwise combinations. These biomarkers, which are described as “core” biomarkers in the Examples below, include IL10, KLK10, KRT19, MUC-16, PTK7, SEZ6L, SPINT1 and TACSTD2. Specifically, it has been found that determining the levels of two biomarkers selected from this list a sample from a subject is particularly effective in detecting, predicting or monitoring ovarian cancer in a subject.

According to a further aspect, the present invention thus provides an in vitro method of detecting, predicting or monitoring ovarian cancer in a subject, said method comprising determining in a sample from said subject the level of two biomarkers selected from the list consisting of IL10, KLK10, KRT19, MUC-16, PTK7, SEZ6L, SPINT1 and TACSTD2 in a subject sample, wherein
(i) the biomarker levels determined are not solely of the biomarkers IL10 and KLK10; 11_10 and MUC-16; KLK10 and MUC-16; MUC-16, IL10 and KLK10, or MUC-16 and KRT19; and
(ii) when the biomarkers are IL10 and KLK10, IL10 and MUC-16 or KLK10 and MUC-16, the level of at least one other biomarker is determined, wherein the at least one other biomarker is (a) from the list or (b) is not solely a kallikrein, CRP, EGF-R, CA-19-9, MIP-1 and/or ferritin; and
(iii) when the biomarkers are MUC-16 and KRT19, the level of at least one other biomarker is determined, wherein the at least one other biomarker is (a) from the list or (b) is not solely (i) FasL and/or M-CSF; (ii) kallikrein 8, CEA and/or M-CSF; (iii) CEA, M-CSF and/or EGFR; or (iv) ErbB2.

In one particular embodiment the biomarker levels determined are not solely of two or more biomarkers selected from the list consisting of IL10, KLK10, MUC-16 and KRT19 (i.e. are not solely any 2, 3 or 4 of IL10, KLK10, MUC-16 and KRT19).

More particularly, according to this aspect of the present invention, when the biomarkers are IL10 and KLK10, IL10 and MUC-16, KLK10 and MUC-16, or KRT19 and MUC-16 (or optionally when the biomarkers are any two, any three or all four of IL10, KLK10, MUC-16 and KRT19), the level of at least one other biomarker is determined, wherein the at least one other biomarker is selected from PTK7, SEZ6L, SPINT1, TACSTD2, CD83, CD99L2, CLEC4D, CLEC6A, CLMP, CPE, ICOSLG, IRF9, LAMP3, MSMB, PROK1, PTK7, SIT1, SKAP1, SPINK5, and TANK, or from, PTK7, SEZ6L, TACSTD2, CD83, CD99L2, CLEC4D, CLEC6A, CLMP, CPE, IRF9, LAMP3, MSMB, PROK1, PTK7, SIT1, SKAP1, SPINK5, and TANK.

It has also been found that particular combinations of biomarkers allow the detection, prediction or monitoring of ovarian cancer with surprisingly good sensitivity and/or selectivity.

In one embodiment, the biomarkers comprise MUC-16 and one of IL10, KLK10, KRT19, PTK7, SEZ6L, SPINT1 and TACSTD2.

According to another aspect, the present invention provides an in vitro method of detecting, predicting or monitoring ovarian cancer in a subject, comprising determining the level of two biomarkers in a sample from said subject, wherein said two biomarkers are:
a) TACSTD2 and SPINT1;
b) TACSTD2 and ICOSLG;
c) SPINT1 and ICOSLG;
d) KLK10 and SPINT1;
e) PTK7 and SPINT1;
f) SEZ6L2 and SPINT1;
g) KRT19 and SPINT1;
h) KRT19 and TACSTD2; or
i) IL10 and SPINT1.

In another aspect, the present invention provides an in vitro method of detecting, predicting or monitoring ovarian cancer in a subject, comprising determining the level of MUC-16 or TACSTD2, and one or more of ICOSLG, CLEC4D or CLEC6A. More particularly, said biomarkers may comprise (i) MUC-16 or TACSTD2, (ii) ICOSLG, and (iii) CLEC4D or CLEC6A.

According to a further aspect, the present invention provides an in vitro method of detecting, predicting or monitoring ovarian cancer in a subject, comprising determining the level of three biomarkers in a sample from said subject, wherein said three biomarkers comprise a first biomarker selected from MUC-16, TACSTD2, and SPINT1 and second and third biomarkers selected from TACSTD2, KRT19, PTK7, SPINT1, MUC-16, KLK10, ICOSLG, IL10, SPINK5, CLEC6A, CLEC4D, CD83, SKAP1 and SEZ6L2, wherein the second and third biomarkers are different from the first biomarker, and from each other, and wherein the biomarker levels determined are not solely of the biomarkers MUC-16, IL10 and KLK10.

The present disclosure also includes sets of reagents to determine the levels of biomarkers in a sample, wherein said biomarkers are selected from the list consisting of CD83, CD99L2, CLEC4D, CLEC6A, CLMP, CPE, FR-alpha, ICOSLG, IL10, IRF9, KLK10, KRT19 LAMP3, MSMB, MUC-16, PROK1, PTK7, SEZ6L2, SIT1, SKAP1, SPINK5, SPINT1, TACSTD2, TANK and WFDC2. Thus, the set may comprise reagents to determine any two or more, e.g. any three or more of the listed biomarkers.

According to one aspect, the present invention provides sets of reagents to determine the levels of biomarkers in a sample, wherein the biomarkers comprise two biomarkers selected from the list consisting of IL10, KLK10, KRT19, MUC-16, PTK7, SEZ6L, SPINT1 and TACSTD2, wherein
(i) the biomarkers are not solely IL10 and KLK10; 11_10 and MUC-16; KLK10 and MUC-16; MUC-16, IL10 and KLK10, or MUC-16 and KRT19; and
(ii) when the biomarkers are IL10 and KLK10, IL10 and MUC-16 or KLK10 and MUC-16, the set of reagents comprises a reagent to determine the level of least one other biomarker, wherein the at least one other biomarker is (a) from the list or (b) is not a kallikrein, CRP, EGF-R, CA-19-9, MIP-1 or ferritin wherein the biomarkers do not solely further comprise a kallikrein, CRP, EGF-R, CA19-9, MIP-1 and/or ferritin; and
(iii) when the biomarkers are MUC-16 and KRT19, the set of reagents comprises a reagent to determine the level of at least one other biomarker, wherein the at least one other biomarker is (a) from the list or (b) is not solely (i) FasL and/or M-CSF; (ii) kallikrein 8, CEA and/or M-CSF; (iii) CEA, M-CSF and/or EGFR; or (iv) ErbB2.

More particularly, the set of reagents does not solely comprise reagents to determine the level of two or more biomarkers selected from the list consisting of IL10, KLK10, MUC-16 and KRT19.

More particularly, when the biomarkers are IL10 and KLK10, IL10 and MUC-16, KLK10 and MUC-16, or KRT19 and MUC-16, (or optionally when the biomarkers are any two, any three or all four of IL10, KLK10, MUC-16 and KRT19), at least one other biomarker is selected from PTK7, SEZ6L, SPINT1, TACSTD2, CD83, CD99L2, CLEC4D, CLEC6A, CLMP, CPE, ICOSLG, IRF9, LAMP3, MSMB, PROK1, PTK7, SIT1, SKAP1, SPINK5, and TANK, or from, PTK7, SEZ6L, TACSTD2, CD83, CD99L2, CLEC4D, CLEC6A, CLMP, CPE, IRF9, LAMP3, MSMB, PROK1, PTK7, SIT1, SKAP1, SPINK5, and TANK.

In one embodiment, the set of reagents may comprise reagents to determine the levels of biomarkers in a sample, wherein said biomarkers comprise MUC-16 and one of IL10, KLK10, KRT19, PTK7, SEZ6L, SPINT1 and TACSTD2.

In a further aspect, the present invention provides sets of reagents to determine the levels of biomarkers in a sample, wherein the biomarkers comprise two biomarkers, wherein the two biomarkers are:
a) TACSTD2 and SPINT1;
b) TACSTD2 and ICOSLG;
c) SPINT1 and ICOSLG;
d) KLK10 and SPINT1;
e) PTK7 and SPINT1;
f) SEZ6L2 and SPINT1;
g) KRT19 and SPINT1;
h) KRT19 and TACSTD2; or
i) IL10 and SPINT1.

In another aspect, a set of reagents may be provided to determine the levels of biomarkers in a sample, wherein the biomarkers are MUC-16 or TACSTD2 and one or more of ICOSLG, CLEC4D or CLEC6A. More particularly, the biomarkers may comprise (i) MUC-16 or TACSTD2, (ii) ICOSLG, and (iii) CLEC4D or CLEC6A.

The present invention also provides a set of reagents to determine the levels of biomarkers in a sample, wherein the biomarkers comprise a first biomarker selected from MUC-16, TACSTD2 and SPINT1 and second and third biomarkers selected from TACSTD2, KRT19, PTK7, SPINT1, MUC-16, KLK10, ICOSLG, IL10, SPINK5, CLEC6A, CLEC4D, CD83, SKAP1 and SEZ6L2, and wherein the second and third biomarkers are different form the first biomarker and from each other, and wherein the biomarkers are not solely MUC-16, IL10 and KLK10.

The present invention also provides test kits and multianalyte panel assays comprising sets of reagents according to the present invention.

As mentioned above and further described in the Examples below, we have identified panels, or combinations, of biomarkers, which are of particular utility in the methods herein. A particular core of markers comprising the core markers MUC-16, SPINT1, TACSTD1 was selected based on further work, and was developed into a 11-plex, or 10-plex panel of biomarkers as described in Example 3 below. These panels were then further developed and investigated, and it was determined that it was not necessary to include all 10 or 11 proteins in the panel. Thus, in further work a 7-plex panel comprising the proteins TACSTD2, PROK1, MSMB, MUC-16, WFDC2, FR-alpha, and KRT19 has been identified, as described in Example 4 below. This 7-plex panel represents an aspect of particular interest.

Accordingly, in another aspect the present disclosure and invention provides a method, particularly an in vitro method, of detecting, predicting or monitoring ovarian cancer in a subject, comprising determining in a sample from said subject the levels of the biomarkers in a panel comprising: TACSTD2, PROK1, MSMB, MUC-16, WFDC2, FR-alpha, and KRT19.

The method may further comprise determining the levels of one or more of the following biomarkers: SPINT1, ICOSLG, CLEC6A, or CDH3.

These additional markers may be added to the 7-plex panel in any number and combination. In other words, any 1, 2, 3 or 4 of the additional markers SPINT1, ICOSLG, CLEC6A, or CDH3 may be included in the panel, in any combination.

In particular embodiments, representative panels of biomarkers may be selected from the following:
(a) TACSTD2, PROK1, MSMB, MUC-16, WFDC2, FR-alpha, KRT19, and SPINT1; or
(b) TACSTD2, PROK1, MSMB, MUC-16, WFDC2, FR-alpha, KRT19, SPINT1 and ICOSLG; or
(c) TACSTD2, PROK1, MSMB, MUC-16, WFDC2, FR-alpha, KRT19, SPINT1, ICOSLG and CLEC6A; or
(d) TACSTD2, PROK1, MSMB, MUC-16, WFDC2, FR-alpha, KRT19, SPINT1, ICOSLG, CLEC6A and CDH3.
   Panel (c) is the 10-plex panel from Example 3, and Panel (d) is the 11-plex panel from Example 3.

More generally, the individual biomarkers of the 7- and 11-plex panels above are of interest as biomarkers for ovarian cancer, and a biomarker panel, or combination, may be made up of a fewer number of members of the 7 plex, or 11-plex panels described above.

Accordingly, in another aspect the present disclosure and invention provides a method, particularly an in vitro method of detecting, predicting or monitoring ovarian cancer in a subject, comprising determining in a sample from said subject the level of two or more biomarkers selected from the list consisting of TACSTD2, PROK1, MSMB, MUC-16, WFDC2, FR-alpha, and KRT19, wherein at least one biomarker is selected from TACSTD2, PROK1 or MSMB.

In an embodiment the biomarker PROK1 is selected. In another embodiment the biomarkers whose levels are determined comprise at least TACSTD2, PROK1 and MSMB. In another embodiment the levels of at least 3, or at least 4, or at least 5, or at least 6 biomarkers are determined.

In an embodiment the biomarkers comprise one or more of MUC-16, WFDC2, FR-alpha, or KRT19. In another embodiment, the biomarkers comprise MUC-16, and optionally one or more of WFDC2, FR-alpha or KRT19. In another embodiment the biomarkers comprise MUC-16, WFDC2, FR-alpha, and KRT19 and at least one biomarker selected from TACSTD2, PROK1 or MSMB. Thus, according to this more general aspect above the levels of a panel of two or more biomarkers may be determined.

In another embodiment, one or more of the following biomarkers may be included in the panel: SPINT1, ICOSLG, CLEC6A, or CDH3. These additional markers may be added to the panel in any number and combination. In other words, any 1, 2, 3 or 4 of the additional markers SPINT1, ICOSLG, CLEC6A, or CDH3 may be included in the panel, in any combination.

In line with these aspects, the present disclosure and invention also provides use of the combination of the proteins TACSTD2, PROK1, MSMB, MUC-16, WFDC2, FR-alpha, and KRT19 as biomarkers for ovarian cancer, optionally further including one or more of the biomarkers SPINT1, ICOSLG, CLEC6A, or CDH3.

Further provided is a set of reagents to determine the levels of biomarkers in a sample, wherein the biomarkers comprise: TACSTD2, PROK1, MSMB, MUC-16, WFDC2, FR-alpha, and KRT19.

Also provided is a set of reagents to determine the levels of biomarkers in a sample, wherein the biomarkers comprise two or more biomarkers selected from the list consisting of TACSTD2, PROK1, MSMB, MUC-16, WFDC2, FR-alpha, and KRT19, and wherein at least one biomarker is TACSTD2, PROK1 or MSMB.

As above, included as part of the present invention and disclosure are test kits and multianalyte panel assays comprising sets of reagents as defined above.

A method of any aspect of the invention may comprise determining the levels of biomarkers in a sample from the subject using a set of reagents as defined herein.

According to another aspect of the invention, in vitro methods of detecting biomarkers in a sample are also provided, wherein said methods comprise determining the level of two or more biomarkers in a sample, wherein said biomarkers are two or more biomarkers as defined herein.

Any of the biomarkers which have been found to be associated with ovarian cancer in the work leading to the present invention may be used in the methods of the present invention. Biomarkers were identified as being associated with ovarian cancer when they were found to account for a portion of the variability between subjects having ovarian cancer and subjects in a control group in a discovery analysis. The level of certain biomarkers determined in samples from at least a subgroup of subjects in a control group was found to differ in samples determined from a patient group having ovarian cancer. Thus, any of the biomarkers mentioned herein are capable of being used as biomarkers for ovarian cancer, either individually, or at least when in combination with one or more other biomarkers, e.g. those disclosed herein. Biomarkers identified during the course of making the present invention may be used singly or in combination with other biomarkers in order to detect, predict or monitor ovarian cancer in a subject. Reference herein to methods comprising the detection of a biomarker in a sample from a subject are not limited to the detection of a single biomarker, and the level of further biomarkers may be determined in conjunction with a named biomarker. Similarly, methods comprising determining the level of two or three biomarkers may comprise the detection of additional biomarkers, and are not limited to only determining the levels of two or three biomarkers recited. The present invention comprises determining the level of biomarkers in a sample from a subject. Reference to selecting a biomarker or biomarkers thus encompasses methods comprising the detection of said biomarker and biomarkers, and should not be understood to be limited to the detection of only the recited biomarkers. Similar considerations also apply to sets of reagents as defined herein.

In certain embodiments, the methods and reagent sets disclosed herein do not comprise determining, or are not for determining, the levels of IL10 and KLK10, IL10 and MUC-16 or KLK10 and MUC-16 alone, i.e. the methods further comprise determining, and the reagent sets are for determining, the level of at least one additional biomarker. Furthermore, for any of these combinations, where the level of an additional biomarker is determined, said additional biomarker is not solely a kallikrein, CRP, EGF-R, CA-19-9, MIP1 and/or ferritin. Thus, at least one additional biomarker is determined which is not a kallikrein, CRP, EGF-R, CA-19-9, MIP1 or any combination thereof.

Furthermore, in certain other embodiments the methods and reagent sets disclosed herein do not encompass or are not for determining the levels of MUC-16 and KRT19 alone, nor determining the levels of MUC-16 and KRT19 and at least one other biomarker, wherein the at least one other biomarker is solely (i) FasL and/or M-CSF; (ii) kallikrein-8, CEA and/or M-CSF; (iii) CEA, M-CSF and/or EGFR; or (iv) ErbB2. In certain embodiments, the at least one other biomarker is not one or more of EGF, G-CSF, IL-6, IL-8, VEGF, MCP-1, anti-IL6, anti-IL8, anti-MUC-16, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-KRT19, EGFR, CEA, kallikrein-8, M-CSF, FasL, ErbB2 and/or Her2/neu. In particular embodiments, the additional biomarker is not solely (i) FasL and M-CSF; (ii) kallikrein-8; (iii) kallikrein-8 and CEA; (iv) kallikrein-8, CEA and M-CSF; (v) CEA, M-CSF and EGFR; or (vi) Erb2.

Further, in some embodiments the methods and reagent sets of the present invention do not comprise determining, or are not for determining, the levels of MUC-16, IL10 and KLK10 alone.

Thus, in some embodiments, where any combination of two, or three, or four of the biomarkers MUC-16, IL10, KLK10 and KRT19 is determined, the methods comprise determining, and the reagent sets are for determining, the level of at least one additional biomarker. Further, in such a case, the additional biomarker(s) do not include solely WFDC2. Thus, methods and reagent sets of the present invention do not encompass determining the level of WFDC2 in combination with IL10 and KLK10, IL10 and MUC-16 or KLK10 and MUC-16, or in combination with MUC-16, IL10 and KLK10, or in combination with MUC-16 and KRT-19, wherein said method does not further comprise determining, or the reagent set is for determining, the level of any additional biomarker.

For any of these combinations, where the level of an additional biomarker is determined, said additional biomarker is not solely a kallikrein, CRP, EGF-R, CA-19-9, MIP1 and/or ferritin as described above. Further, in another embodiment it is not solely EGF, G-CSF, IL-6, IL-8, VEGF, MCP-1, anti-IL6, anti-IL8, anti-MUC-16, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-KRT19, EGFR, CEA, kallikrein-8, M-CSF, FasL, ErbB2 and/or Her2/neu.

Thus, in certain representative embodiments, the methods of the present invention may further comprise determining, and the reagent sets may be for further determining, the level of an additional biomarker, wherein said additional biomarker is CD83, CD99L2, CLEC4D, CLEC6A, CLMP, CPE, ICOSLG, IRF9, LAMP3, MSMB, PROK1, PTK7, SEZ6L2, SIT1, SKAP1, SPINK5, SPINT1, TACSTD2, or TANK. CDH3 may also be an additional biomarker, as demonstrated in Example 3 below.

In further representative embodiments, the methods and reagent sets of the present invention may additionally not comprise or be for the detection of SPINT1 and/or ICOSLG in combination with any one of IL10, KLK10, MUC-16 or KRT19 or any combination thereof, wherein the level of no further biomarkers is determined. Thus, according to certain embodiments, the present invention may further not include determining the level of ICOSLG and any one of IL10, KLK10, MUC-16, or KRT19 or any combination thereof, or determining the level of SPINT1 and any one of IL10, KLK10, MUC-16, or KRT19 or any combination thereof, or determining the level of ICOSLG and SPINT1 and any one of IL10, KLK10, MUC-16 or KRT 19 or any combination thereof, wherein solely the levels of said biomarkers are determined. Thus, in certain embodiments, any methods or reagent sets which may comprise or be for determining the level of any of these combinations of biomarkers alone further comprise determining the level of an additional biomarker, e.g. wherein said additional biomarker is any other biomarker identified herein. However, where said methods comprise determining the level of an additional biomarker, said additional biomarker is not solely a kallikrein, CRP, EGF-R, CA-19-9, MIP1 and/or ferritin as described above, and/or not solely EGF, G-CSF, IL-6, IL-8, VEGF, MCP-1, anti-IL6, anti-IL8, anti-MUC-16, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-KRT19, EGFR, CEA, kallikrein-8, M-CSF, FasL, ErbB2 and/or Her2/neu.

In further representative embodiments, the methods and reagent sets of the present invention may additionally not comprise or be for the detection of TACSTD2 in combination with any one of IL10, KLK10, MUC-16 or KRT19 or any combination thereof, wherein the level of no further biomarkers is determined. Thus, according to certain embodiments, the present invention may further not include determining the level of TACSTD2 and any one of IL10, KLK10, MUC-16, or KRT19 or any combination thereof, wherein solely the levels of said biomarkers are determined. Thus, in certain embodiments, any methods or reagent sets which may comprise or be for determining the level of any of these combinations of biomarkers alone further comprise determining the level of an additional biomarker, e.g. wherein said additional biomarker is any other biomarker identified herein. However, where said methods comprise determining the level of an additional biomarker, said additional biomarker is not solely a kallikrein, CRP, EGF-R, CA-19-9, MIP1 and/or ferritin as described above, and/or is not solely EGF, G-CSF, IL-6, IL-8, VEGF, MCP-1, anti-IL6, anti-IL8, anti-MUC-16, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-KRT19, EGFR, CEA, kallikrein-8, M-CSF, FasL, ErbB2 and/or Her2/neu.

Put another way, when the biomarkers are IL10 and KLK10, IL10 and MUC-16, KLK10 and MUC-16, MUC-16, IL10 and KLK10, or MUC-16 and KRT19 (or, alternatively, any one or more of MUC-16, IL10, KLK10, or KRT19) and the level of at least one other biomarker is determined, in certain representative embodiments said additional biomarker may not be ICOSLG and/or SPINT1 and/or TACSTD2, wherein the method comprises determining the level solely of said biomarkers or the set of reagents comprises reagents for the detection of said biomarkers alone.

Furthermore, in certain additional representative embodiments, the biomarker levels determined are not solely of two or more of the biomarkers MUC-16, IL10, KLK10, KRT19, SPINT1, ICOSLG and TACSTD2.

Thus, in certain embodiments, the level of an additional biomarker may be determined, or the reagent set may further comprise a reagent for determining the level of an additional biomarker, wherein said additional biomarker is CD83, CD99L2, CLEC4D, CLEC6A, CLMP, CPE, ICOSLG, IRF9, LAMP3, MSMB, PROK1, PTK7, SEZ6L2, SIT1, SKAP1, SPINK5, TACSTD2 or TANK. In other embodiments, the additional biomarker is CD83, CD99L2, CLEC4D, CLEC6A, CLMP, CPE, IRF9, LAMP3, MSMB, PROK1, PTK7, SEZ6L2, SIT1, SKAP1, SPINK5, SPINT1, TACSTD2 or TANK. In yet further embodiments, the additional biomarker is CD83, CD99L2, CLEC4D, CLEC6A, CLMP, CPE, FR-alpha, IRF9, LAMP3, MSMB, PROK1, PTK7, SEZ6L2, SIT1, SKAP1, SPINK5, TACSTD2 or TANK.

In yet other representative embodiments, the present invention may not comprise the detection of, and sets of reagents may not comprise reagents to determine the level of, TACSTD2 and/or MSMB in combination with any one of IL10, KLK10, MUC-16 or KRT19 or any combination thereof, wherein the level of no further biomarkers is determined or wherein the set of reagents does not comprise reagents to determine the level of further biomarkers. Thus, according to certain embodiments, the present invention may further not include determining the level of MSMB and any one of IL10, KLK10, or MUC-16 or KRT19 or any combination thereof, or determining the level of TACSTD2 and any one of IL10, KLK10, MUC-16 or KRT19 or any combination thereof, or determining the level of MSMB and TACSTD2 and any one of IL10, KLK10, MUC-16, or KRT19 or any combination thereof, wherein solely the levels of said biomarkers are determined. Correspondingly, sets of reagents may not comprise reagents to determine the levels of said biomarkers wherein the sets of reagents do not comprise reagents to determine the levels of further biomarkers. Thus, in certain embodiments, any methods or reagent sets which may comprise or be for determining the level of any of these combinations of biomarkers alone may further comprise determining the level of an additional biomarker, e.g. wherein said additional biomarker is any other biomarker identified herein. However, where said methods or reagent sets comprise or are for determining the level of an additional biomarker, said additional biomarker is not solely a kallikrein, CRP, EGF-R, CA-19-9, MIP1 and/or ferritin as described above and/or is not solely EGF, G-CSF, IL-6, IL-8, VEGF, MCP-1, anti-IL6, anti-IL8, anti-MUC-16, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-KRT19, EGFR, CEA, kallikrein-8, M-CSF, FasL, ErbB2 and/or Her2/neu.

Thus, in certain embodiments, the additional biomarker is CD83, CD99L2, CLEC4D, CLEC6A, CLMP, CPE, ICOSLG, IRF9, LAMP3, MSMB, PROK1, PTK7, SEZ6L2, SIT1, SKAP1, SPINK5, SPINT1, TACSTD2 or TANK. In other embodiments, the additional biomarker is CD83, CD99L2, CLEC4D, CLEC6A, CLMP, CPE, ICOSLG, IRF9, LAMP3, PROK1, PTK7, SEZ6L2, SIT1, SKAP1, SPINK5, SPINT1, TACSTD2 or TANK. In yet further embodiments, the additional biomarker is CD83, CD99L2, CLEC4D, CLEC6A, CLMP, CPE, ICOSLG, IRF9, LAMP3, MSMB, PROK1, PTK7, SEZ6L2, SIT1, SKAP1, SPINK5, SPINT1 or TANK. In yet another embodiment, the additional biomarker is CD83, CD99L2, CLEC4D, CLEC6A, CLMP, CPE, ICOSLG, IRF9, LAMP3, MUC-16, PROK1, PTK7, SEZ6L2, SIT1, SKAP1, SPINK5, or TANK. Thus, in one representative embodiment, the additional biomarker is CD83, CD99L2, CLEC4D, CLEC6A, CLMP, CPE, IRF9, LAMP3, PROK1, PTK7, SEZ6L2, SIT1, SKAP1, SPINK5 or TANK.

We have identified that the use of MUC-16 as a biomarker for ovarian cancer in combination with other biomarkers was particularly useful in detecting, predicting or monitoring ovarian cancer in a subject, and in particular that such combinations provided a surprising improvement in the sensitivity and specific of the detection of ovarian cancer than the use of MUC-16 alone. Certain embodiments of the method of the present invention therefore optionally comprise detecting the level of MUC-16 in a sample, in addition to another biomarker or other biomarkers recited herein.

In particular embodiments of the present invention, the methods of the present invention, and the sets of reagents to determine the levels of biomarkers in a sample may comprise determining the levels of, or comprise reagents to determine the levels of MUC-16 and one of IL10, KLK10, KRT19, PTK7, SEZ6L, SPINT1 and TACSTD2, i.e. the biomarkers may comprise MUC-16 and one of IL10, KLK10, KRT19, PTK7, SEZ6L, SPINT1 and TACSTD2.

Any of the methods according to the present invention for detecting, predicting or monitoring ovarian cancer in a subject, or the sets of reagents to determine the levels of biomarkers in a sample may further comprise determining or may further comprise a reagent to determine the level of at least one additional biomarker selected from the list consisting of CD83, CD99L2, CLEC4D, CLEC6A, CLMP, CPE, FR-alpha, ICOSLG, IL10, IRF9, KLK10, KRT19, LAMP3, MSMB, MUC-16, PROK1, PTK7, SEZ6L2, SIT1, SKAP1, SPINK5, SPINT1, TACSTD2, TANK and WFDC2 in said sample. Thus, according to particular representative embodiments, the methods may comprise determining the level of one further biomarker selected from this list and the sets of reagents may comprise a reagent to determine the level of one further biomarker selected from this list. More particularly, such methods may comprise determining the level of at least two biomarkers, or more particularly, at least three biomarkers selected from this list. Determining the level of further biomarkers (e.g. four five, six, seven, eight, nine, ten or more biomarkers form this list) are also contemplated. Sets of reagents may similarly comprise reagents to determine the level of at least two biomarkers, or more particularly, at least three biomarkers selected from this list, or four five, six, seven, eight, nine, ten or more biomarkers from this list.

In certain embodiments, the method may comprise determining the level of two biomarkers in a sample from a subject, wherein said two biomarkers are:
a) KLK10 and CPE;
b) PTK7 and ICOSLG;
c) SEZ6L2 and ICOSLG;
d) KRT19 and CLEC4D; or
e) IL10 and ICOSLG.

The methods of the invention may comprise determining the level of any three biomarkers which have been identified in the work leading to the present invention to be associated with ovarian cancer, in any combination. A complete list of the possible three-member biomarker panels made up of the biomarkers which have been identified is provided in Table 2. In certain embodiments, the methods of the present invention may comprise determining the level of three biomarkers, wherein said three biomarkers are any set of three biomarkers provided in Table 2.

TABLE 2
three-member biomarker panels

| Marker 1 | Marker 2 | Marker 3 |
|---|---|---|
| IL10 | KLK10 | KRT19 |
| IL10 | KLK10 | MUC-16 |
| IL10 | KLK10 | PTK7 |
| IL10 | KLK10 | SEZ6L2 |
| IL10 | KLK10 | SPINT1 |
| IL10 | KLK10 | TACSTD2 |
| IL10 | KLK10 | CD83 |
| IL10 | KLK10 | CD99L2 |
| IL10 | KLK10 | CLEC4D |
| IL10 | KLK10 | CLEC6A |
| IL10 | KLK10 | CLMP |
| IL10 | KLK10 | CPE |
| IL10 | KLK10 | FR-alpha |
| IL10 | KLK10 | ICOSLG |
| IL10 | KLK10 | IRF9 |
| IL10 | KLK10 | LAMP3 |
| IL10 | KLK10 | MSMB |
| IL10 | KLK10 | PROK1 |
| IL10 | KLK10 | SIT1 |
| IL10 | KLK10 | SKAP1 |
| IL10 | KLK10 | SPINK5 |
| IL10 | KLK10 | TANK |
| IL10 | KLK10 | WFDC2 |
| IL10 | KRT19 | MUC-16 |
| IL10 | KRT19 | PTK7 |
| IL10 | KRT19 | SEZ6L2 |
| IL10 | KRT19 | SPINT1 |
| IL10 | KRT19 | TACSTD2 |
| IL10 | KRT19 | CD83 |
| IL10 | KRT19 | CD99L2 |
| IL10 | KRT19 | CLEC4D |
| IL10 | KRT19 | CLEC6A |
| IL10 | KRT19 | CLMP |
| IL10 | KRT19 | CPE |
| IL10 | KRT19 | FR-alpha |
| IL10 | KRT19 | ICOSLG |
| IL10 | KRT19 | IRF9 |
| IL10 | KRT19 | LAMP3 |
| IL10 | KRT19 | MSMB |
| IL10 | KRT19 | PROK1 |
| IL10 | KRT19 | SIT1 |
| IL10 | KRT19 | SKAP1 |
| IL10 | KRT19 | SPINK5 |
| IL10 | KRT19 | TANK |
| IL10 | KRT19 | WFDC2 |
| IL10 | MUC-16 | PTK7 |
| IL10 | MUC-16 | SEZ6L2 |
| IL10 | MUC-16 | SPINT1 |
| IL10 | MUC-16 | TACSTD2 |
| IL10 | MUC-16 | CD83 |
| IL10 | MUC-16 | CD99L2 |
| IL10 | MUC-16 | CLEC4D |
| IL10 | MUC-16 | CLEC6A |
| IL10 | MUC-16 | CLMP |
| IL10 | MUC-16 | CPE |
| IL10 | MUC-16 | FR-alpha |
| IL10 | MUC-16 | ICOSLG |
| IL10 | MUC-16 | IRF9 |
| IL10 | MUC-16 | LAMP3 |
| IL10 | MUC-16 | MSMB |
| IL10 | MUC-16 | PROK1 |
| IL10 | MUC-16 | SIT1 |
| IL10 | MUC-16 | SKAP1 |
| IL10 | MUC-16 | SPINK5 |
| IL10 | MUC-16 | TANK |
| IL10 | MUC-16 | WFDC2 |
| IL10 | PTK7 | SEZ6L2 |
| IL10 | PTK7 | SPINT1 |
| IL10 | PTK7 | TACSTD2 |
| IL10 | PTK7 | CD83 |
| IL10 | PTK7 | CD99L2 |
| IL10 | PTK7 | CLEC4D |
| IL10 | PTK7 | CLEC6A |
| IL10 | PTK7 | CLMP |
| IL10 | PTK7 | CPE |
| IL10 | PTK7 | FR-alpha |
| IL10 | PTK7 | ICOSLG |
| IL10 | PTK7 | IRF9 |
| IL10 | PTK7 | LAMP3 |
| IL10 | PTK7 | MSMB |
| IL10 | PTK7 | PROK1 |
| IL10 | PTK7 | SIT1 |
| IL10 | PTK7 | SKAP1 |
| IL10 | PTK7 | SPINK5 |
| IL10 | PTK7 | TANK |
| IL10 | PTK7 | WFDC2 |
| IL10 | SEZ6L2 | SPINT1 |
| IL10 | SEZ6L2 | TACSTD2 |
| IL10 | SEZ6L2 | CD83 |
| IL10 | SEZ6L2 | CD99L2 |
| IL10 | SEZ6L2 | CLEC4D |
| IL10 | SEZ6L2 | CLEC6A |
| IL10 | SEZ6L2 | CLMP |
| IL10 | SEZ6L2 | CPE |
| IL10 | SEZ6L2 | FR-alpha |
| IL10 | SEZ6L2 | ICOSLG |
| IL10 | SEZ6L2 | IRF9 |
| IL10 | SEZ6L2 | LAMP3 |
| IL10 | SEZ6L2 | MSMB |
| IL10 | SEZ6L2 | PROK1 |
| IL10 | SEZ6L2 | SIT1 |
| IL10 | SEZ6L2 | SKAP1 |
| IL10 | SEZ6L2 | SPINK5 |
| IL10 | SEZ6L2 | TANK |
| IL10 | SEZ6L2 | WFDC2 |
| IL10 | SPINT1 | TACSTD2 |
| IL10 | SPINT1 | CD83 |
| IL10 | SPINT1 | CD99L2 |
| IL10 | SPINT1 | CLEC4D |
| IL10 | SPINT1 | CLEC6A |
| IL10 | SPINT1 | CLMP |
| IL10 | SPINT1 | CPE |
| IL10 | SPINT1 | FR-alpha |
| IL10 | SPINT1 | ICOSLG |
| IL10 | SPINT1 | IRF9 |
| IL10 | SPINT1 | LAMP3 |
| IL10 | SPINT1 | MSMB |
| IL10 | SPINT1 | PROK1 |
| IL10 | SPINT1 | SIT1 |
| IL10 | SPINT1 | SKAP1 |
| IL10 | SPINT1 | SPINK5 |
| IL10 | SPINT1 | TANK |
| IL10 | SPINT1 | WFDC2 |
| IL10 | TACSTD2 | CD83 |
| IL10 | TACSTD2 | CD99L2 |
| IL10 | TACSTD2 | CLEC4D |
| IL10 | TACSTD2 | CLEC6A |
| IL10 | TACSTD2 | CLMP |
| IL10 | TACSTD2 | CPE |
| IL10 | TACSTD2 | FR-alpha |
| IL10 | TACSTD2 | ICOSLG |
| IL10 | TACSTD2 | IRF9 |
| IL10 | TACSTD2 | LAMP3 |
| IL10 | TACSTD2 | MSMB |
| IL10 | TACSTD2 | PROK1 |
| IL10 | TACSTD2 | SIT1 |
| IL10 | TACSTD2 | SKAP1 |
| IL10 | TACSTD2 | SPINK5 |
| IL10 | TACSTD2 | TANK |
| IL10 | TACSTD2 | WFDC2 |
| IL10 | CD83 | CD99L2 |
| IL10 | CD83 | CLEC4D |
| IL10 | CD83 | CLEC6A |
| IL10 | CD83 | CLMP |
| IL10 | CD83 | CPE |
| IL10 | CD83 | FR-alpha |
| IL10 | CD83 | ICOSLG |
| IL10 | CD83 | IRF9 |
| IL10 | CD83 | LAMP3 |
| IL10 | CD83 | MSMB |
| IL10 | CD83 | PROK1 |
| IL10 | CD83 | SIT1 |
| IL10 | CD83 | SKAP1 |
| IL10 | CD83 | SPINK5 |
| IL10 | CD83 | TANK |
| IL10 | CD83 | WFDC2 |
| IL10 | CD99L2 | CLEC4D |
| IL10 | CD99L2 | CLEC6A |
| IL10 | CD99L2 | CLMP |
| IL10 | CD99L2 | CPE |
| IL10 | CD99L2 | FR-alpha |
| IL10 | CD99L2 | ICOSLG |
| IL10 | CD99L2 | IRF9 |
| IL10 | CD99L2 | LAMP3 |
| IL10 | CD99L2 | MSMB |
| IL10 | CD99L2 | PROK1 |
| IL10 | CD99L2 | SIT1 |
| IL10 | CD99L2 | SKAP1 |
| IL10 | CD99L2 | SPINK5 |
| IL10 | CD99L2 | TANK |
| IL10 | CD99L2 | WFDC2 |
| IL10 | CLEC4D | CLEC6A |
| IL10 | CLEC4D | CLMP |
| IL10 | CLEC4D | CPE |
| IL10 | CLEC4D | FR-alpha |
| IL10 | CLEC4D | ICOSLG |
| IL10 | CLEC4D | IRF9 |
| IL10 | CLEC4D | LAMP3 |
| IL10 | CLEC4D | MSMB |
| IL10 | CLEC4D | PROK1 |
| IL10 | CLEC4D | SIT1 |
| IL10 | CLEC4D | SKAP1 |
| IL10 | CLEC4D | SPINK5 |
| IL10 | CLEC4D | TANK |
| IL10 | CLEC4D | WFDC2 |
| IL10 | CLEC6A | CLMP |
| IL10 | CLEC6A | CPE |
| IL10 | CLEC6A | FR-alpha |
| IL10 | CLEC6A | ICOSLG |
| IL10 | CLEC6A | IRF9 |
| IL10 | CLEC6A | LAMP3 |
| IL10 | CLEC6A | MSMB |
| IL10 | CLEC6A | PROK1 |
| IL10 | CLEC6A | SIT1 |
| IL10 | CLEC6A | SKAP1 |
| IL10 | CLEC6A | SPINK5 |
| IL10 | CLEC6A | TANK |
| IL10 | CLEC6A | WFDC2 |
| IL10 | CLMP | CPE |
| IL10 | CLMP | FR-alpha |
| IL10 | CLMP | ICOSLG |
| IL10 | CLMP | IRF9 |
| IL10 | CLMP | LAMP3 |
| IL10 | CLMP | MSMB |
| IL10 | CLMP | PROK1 |
| IL10 | CLMP | SIT1 |
| IL10 | CLMP | SKAP1 |
| IL10 | CLMP | SPINK5 |
| IL10 | CLMP | TANK |
| IL10 | CLMP | WFDC2 |
| IL10 | CPE | FR-alpha |
| IL10 | CPE | ICOSLG |
| IL10 | CPE | IRF9 |
| IL10 | CPE | LAMP3 |
| IL10 | CPE | MSMB |
| IL10 | CPE | PROK1 |
| IL10 | CPE | SIT1 |
| IL10 | CPE | SKAP1 |
| IL10 | CPE | SPINK5 |
| IL10 | CPE | TANK |
| IL10 | CPE | WFDC2 |
| IL10 | FR-alpha | ICOSLG |
| IL10 | FR-alpha | IRF9 |
| IL10 | FR-alpha | LAMP3 |
| IL10 | FR-alpha | MSMB |
| IL10 | FR-alpha | PROK1 |
| IL10 | FR-alpha | SIT1 |
| IL10 | FR-alpha | SKAP1 |
| IL10 | FR-alpha | SPINK5 |
| IL10 | FR-alpha | TANK |
| IL10 | FR-alpha | WFDC2 |
| IL10 | ICOSLG | IRF9 |
| IL10 | ICOSLG | LAMP3 |
| IL10 | ICOSLG | MSMB |
| IL10 | ICOSLG | PROK1 |
| IL10 | ICOSLG | SIT1 |
| IL10 | ICOSLG | SKAP1 |
| IL10 | ICOSLG | SPINK5 |
| IL10 | ICOSLG | TANK |
| IL10 | ICOSLG | WFDC2 |
| IL10 | IRF9 | LAMP3 |
| IL10 | IRF9 | MSMB |
| IL10 | IRF9 | PROK1 |
| IL10 | IRF9 | SIT1 |
| IL10 | IRF9 | SKAP1 |
| IL10 | IRF9 | SPINK5 |
| IL10 | IRF9 | TANK |
| IL10 | IRF9 | WFDC2 |
| IL10 | LAMP3 | MSMB |
| IL10 | LAMP3 | PROK1 |
| IL10 | LAMP3 | SIT1 |
| IL10 | LAMP3 | SKAP1 |
| IL10 | LAMP3 | SPINK5 |
| IL10 | LAMP3 | TANK |
| IL10 | LAMP3 | WFDC2 |
| IL10 | MSMB | PROK1 |
| IL10 | MSMB | SIT1 |
| IL10 | MSMB | SKAP1 |
| IL10 | MSMB | SPINK5 |
| IL10 | MSMB | TANK |
| IL10 | MSMB | WFDC2 |
| IL10 | PROK1 | SIT1 |
| IL10 | PROK1 | SKAP1 |
| IL10 | PROK1 | SPINK5 |
| IL10 | PROK1 | TANK |
| IL10 | PROK1 | WFDC2 |
| IL10 | SIT1 | SKAP1 |
| IL10 | SIT1 | SPINK5 |
| IL10 | SIT1 | TANK |
| IL10 | SIT1 | WFDC2 |
| IL10 | SKAP1 | SPINK5 |
| IL10 | SKAP1 | TANK |
| IL10 | SKAP1 | WFDC2 |
| IL10 | SPINK5 | TANK |
| IL10 | SPINK5 | WFDC2 |
| IL10 | TANK | WFDC2 |
| KLK10 | KRT19 | MUC-16 |
| KLK10 | KRT19 | PTK7 |
| KLK10 | KRT19 | SEZ6L2 |
| KLK10 | KRT19 | SPINT1 |
| KLK10 | KRT19 | TACSTD2 |
| KLK10 | KRT19 | CD83 |
| KLK10 | KRT19 | CD99L2 |
| KLK10 | KRT19 | CLEC4D |
| KLK10 | KRT19 | CLEC6A |
| KLK10 | KRT19 | CLMP |
| KLK10 | KRT19 | CPE |
| KLK10 | KRT19 | FR-alpha |
| KLK10 | KRT19 | ICOSLG |
| KLK10 | KRT19 | IRF9 |
| KLK10 | KRT19 | LAMP3 |
| KLK10 | KRT19 | MSMB |
| KLK10 | KRT19 | PROK1 |
| KLK10 | KRT19 | SIT1 |
| KLK10 | KRT19 | SKAP1 |
| KLK10 | KRT19 | SPINK5 |
| KLK10 | KRT19 | TANK |
| KLK10 | KRT19 | WFDC2 |
| KLK10 | MUC-16 | PTK7 |
| KLK10 | MUC-16 | SEZ6L2 |
| KLK10 | MUC-16 | SPINT1 |
| KLK10 | MUC-16 | TACSTD2 |
| KLK10 | MUC-16 | CD83 |
| KLK10 | MUC-16 | CD99L2 |
| KLK10 | MUC-16 | CLEC4D |
| KLK10 | MUC-16 | CLEC6A |
| KLK10 | MUC-16 | CLMP |
| KLK10 | MUC-16 | CPE |
| KLK10 | MUC-16 | FR-alpha |
| KLK10 | MUC-16 | ICOSLG |
| KLK10 | MUC-16 | IRF9 |
| KLK10 | MUC-16 | LAMP3 |
| KLK10 | MUC-16 | MSMB |
| KLK10 | MUC-16 | PROK1 |
| KLK10 | MUC-16 | SIT1 |
| KLK10 | MUC-16 | SKAP1 |
| KLK10 | MUC-16 | SPINK5 |
| KLK10 | MUC-16 | TANK |
| KLK10 | MUC-16 | WFDC2 |
| KLK10 | PTK7 | SEZ6L2 |
| KLK10 | PTK7 | SPINT1 |
| KLK10 | PTK7 | TACSTD2 |
| KLK10 | PTK7 | CD83 |
| KLK10 | PTK7 | CD99L2 |
| KLK10 | PTK7 | CLEC4D |
| KLK10 | PTK7 | CLEC6A |
| KLK10 | PTK7 | CLMP |
| KLK10 | PTK7 | CPE |
| KLK10 | PTK7 | FR-alpha |
| KLK10 | PTK7 | ICOSLG |
| KLK10 | PTK7 | IRF9 |
| KLK10 | PTK7 | LAMP3 |
| KLK10 | PTK7 | MSMB |
| KLK10 | PTK7 | PROK1 |
| KLK10 | PTK7 | SIT1 |
| KLK10 | PTK7 | SKAP1 |
| KLK10 | PTK7 | SPINK5 |
| KLK10 | PTK7 | TANK |
| KLK10 | PTK7 | WFDC2 |
| KLK10 | SEZ6L2 | SPINT1 |
| KLK10 | SEZ6L2 | TACSTD2 |
| KLK10 | SEZ6L2 | CD83 |
| KLK10 | SEZ6L2 | CD99L2 |
| KLK10 | SEZ6L2 | CLEC4D |
| KLK10 | SEZ6L2 | CLEC6A |
| KLK10 | SEZ6L2 | CLMP |
| KLK10 | SEZ6L2 | CPE |
| KLK10 | SEZ6L2 | FR-alpha |
| KLK10 | SEZ6L2 | ICOSLG |
| KLK10 | SEZ6L2 | IRF9 |
| KLK10 | SEZ6L2 | LAMP3 |
| KLK10 | SEZ6L2 | MSMB |
| KLK10 | SEZ6L2 | PROK1 |
| KLK10 | SEZ6L2 | SIT1 |
| KLK10 | SEZ6L2 | SKAP1 |
| KLK10 | SEZ6L2 | SPINK5 |
| KLK10 | SEZ6L2 | TANK |
| KLK10 | SEZ6L2 | WFDC2 |
| KLK10 | SPINT1 | TACSTD2 |
| KLK10 | SPINT1 | CD83 |
| KLK10 | SPINT1 | CD99L2 |
| KLK10 | SPINT1 | CLEC4D |
| KLK10 | SPINT1 | CLEC6A |
| KLK10 | SPINT1 | CLMP |
| KLK10 | SPINT1 | CPE |
| KLK10 | SPINT1 | FR-alpha |
| KLK10 | SPINT1 | ICOSLG |
| KLK10 | SPINT1 | IRF9 |
| KLK10 | SPINT1 | LAMP3 |
| KLK10 | SPINT1 | MSMB |
| KLK10 | SPINT1 | PROK1 |
| KLK10 | SPINT1 | SIT1 |
| KLK10 | SPINT1 | SKAP1 |
| KLK10 | SPINT1 | SPINK5 |
| KLK10 | SPINT1 | TANK |
| KLK10 | SPINT1 | WFDC2 |
| KLK10 | TACSTD2 | CD83 |
| KLK10 | TACSTD2 | CD99L2 |
| KLK10 | TACSTD2 | CLEC4D |
| KLK10 | TACSTD2 | CLEC6A |
| KLK10 | TACSTD2 | CLMP |
| KLK10 | TACSTD2 | CPE |
| KLK10 | TACSTD2 | FR-alpha |
| KLK10 | TACSTD2 | ICOSLG |
| KLK10 | TACSTD2 | IRF9 |
| KLK10 | TACSTD2 | LAMP3 |
| KLK10 | TACSTD2 | MSMB |
| KLK10 | TACSTD2 | PROK1 |
| KLK10 | TACSTD2 | SIT1 |
| KLK10 | TACSTD2 | SKAP1 |
| KLK10 | TACSTD2 | SPIN KS |
| KLK10 | TACSTD2 | TANK |
| KLK10 | TACSTD2 | WFDC2 |
| KLK10 | CD83 | CD99L2 |
| KLK10 | CD83 | CLEC4D |
| KLK10 | CD83 | CLEC6A |
| KLK10 | CD83 | CLMP |
| KLK10 | CD83 | CPE |
| KLK10 | CD83 | FR-alpha |
| KLK10 | CD83 | ICOSLG |
| KLK10 | CD83 | IRF9 |
| KLK10 | CD83 | LAMP3 |
| KLK10 | CD83 | MSMB |
| KLK10 | CD83 | PROK1 |
| KLK10 | CD83 | SIT1 |
| KLK10 | CD83 | SKAP1 |
| KLK10 | CD83 | SPINK5 |
| KLK10 | CD83 | TANK |
| KLK10 | CD83 | WFDC2 |
| KLK10 | CD99L2 | CLEC4D |
| KLK10 | CD99L2 | CLEC6A |
| KLK10 | CD99L2 | CLMP |
| KLK10 | CD99L2 | CPE |
| KLK10 | CD99L2 | FR-alpha |
| KLK10 | CD99L2 | ICOSLG |
| KLK10 | CD99L2 | IRF9 |
| KLK10 | CD99L2 | LAMP3 |
| KLK10 | CD99L2 | MSMB |
| KLK10 | CD99L2 | PROK1 |
| KLK10 | CD99L2 | SIT1 |
| KLK10 | CD99L2 | SKAP1 |
| KLK10 | CD99L2 | SPINK5 |
| KLK10 | CD99L2 | TANK |
| KLK10 | CD99L2 | WFDC2 |
| KLK10 | CLEC4D | CLEC6A |
| KLK10 | CLEC4D | CLMP |
| KLK10 | CLEC4D | CPE |
| KLK10 | CLEC4D | FR-alpha |
| KLK10 | CLEC4D | ICOSLG |
| KLK10 | CLEC4D | IRF9 |
| KLK10 | CLEC4D | LAMP3 |
| KLK10 | CLEC4D | MSMB |
| KLK10 | CLEC4D | PROK1 |
| KLK10 | CLEC4D | SIT1 |
| KLK10 | CLEC4D | SKAP1 |
| KLK10 | CLEC4D | SPINK5 |
| KLK10 | CLEC4D | TANK |
| KLK10 | CLEC4D | WFDC2 |
| KLK10 | CLEC6A | CLMP |
| KLK10 | CLEC6A | CPE |
| KLK10 | CLEC6A | FR-alpha |
| KLK10 | CLEC6A | ICOSLG |
| KLK10 | CLEC6A | IRF9 |
| KLK10 | CLEC6A | LAMP3 |
| KLK10 | CLEC6A | MSMB |
| KLK10 | CLEC6A | PROK1 |
| KLK10 | CLEC6A | SIT1 |
| KLK10 | CLEC6A | SKAP1 |
| KLK10 | CLEC6A | SPINK5 |
| KLK10 | CLEC6A | TANK |
| KLK10 | CLEC6A | WFDC2 |
| KLK10 | CLMP | CPE |
| KLK10 | CLMP | FR-alpha |
| KLK10 | CLMP | ICOSLG |
| KLK10 | CLMP | IRF9 |
| KLK10 | CLMP | LAMP3 |
| KLK10 | CLMP | MSMB |
| KLK10 | CLMP | PROK1 |
| KLK10 | CLMP | SIT1 |
| KLK10 | CLMP | SKAP1 |
| KLK10 | CLMP | SPINK5 |
| KLK10 | CLMP | TANK |
| KLK10 | CLMP | WFDC2 |
| KLK10 | CPE | FR-alpha |
| KLK10 | CPE | ICOSLG |
| KLK10 | CPE | IRF9 |
| KLK10 | CPE | LAMP3 |
| KLK10 | CPE | MSMB |
| KLK10 | CPE | PROK1 |
| KLK10 | CPE | SIT1 |
| KLK10 | CPE | SKAP1 |
| KLK10 | CPE | SPINK5 |
| KLK10 | CPE | TANK |
| KLK10 | CPE | WFDC2 |
| KLK10 | FR-alpha | ICOSLG |
| KLK10 | FR-alpha | IRF9 |
| KLK10 | FR-alpha | LAMP3 |
| KLK10 | FR-alpha | MSMB |
| KLK10 | FR-alpha | PROK1 |
| KLK10 | FR-alpha | SIT1 |
| KLK10 | FR-alpha | SKAP1 |
| KLK10 | FR-alpha | SPINK5 |
| KLK10 | FR-alpha | TANK |
| KLK10 | FR-alpha | WFDC2 |
| KLK10 | ICOSLG | IRF9 |
| KLK10 | ICOSLG | LAMP3 |
| KLK10 | ICOSLG | MSMB |
| KLK10 | ICOSLG | PROK1 |
| KLK10 | ICOSLG | SIT1 |
| KLK10 | ICOSLG | SKAP1 |
| KLK10 | ICOSLG | SPINK5 |
| KLK10 | ICOSLG | TANK |
| KLK10 | ICOSLG | WFDC2 |
| KLK10 | IRF9 | LAMP3 |
| KLK10 | IRF9 | MSMB |
| KLK10 | IRF9 | PROK1 |
| KLK10 | IRF9 | SIT1 |
| KLK10 | IRF9 | SKAP1 |
| KLK10 | IRF9 | SPINK5 |
| KLK10 | IRF9 | TANK |
| KLK10 | IRF9 | WFDC2 |
| KLK10 | LAMP3 | MSMB |
| KLK10 | LAMP3 | PROK1 |
| KLK10 | LAMP3 | SIT1 |
| KLK10 | LAMP3 | SKAP1 |
| KLK10 | LAMP3 | SPINK5 |
| KLK10 | LAMP3 | TANK |
| KLK10 | LAMP3 | WFDC2 |
| KLK10 | MSMB | PROK1 |
| KLK10 | MSMB | SIT1 |
| KLK10 | MSMB | SKAP1 |
| KLK10 | MSMB | SPINK5 |
| KLK10 | MSMB | TANK |
| KLK10 | MSMB | WFDC2 |
| KLK10 | PROK1 | SIT1 |
| KLK10 | PROK1 | SKAP1 |
| KLK10 | PROK1 | SPINK5 |
| KLK10 | PROK1 | TANK |
| KLK10 | PROK1 | WFDC2 |
| KLK10 | SIT1 | SKAP1 |
| KLK10 | SIT1 | SPINK5 |
| KLK10 | SIT1 | TANK |
| KLK10 | SIT1 | WFDC2 |
| KLK10 | SKAP1 | SPINK5 |
| KLK10 | SKAP1 | TANK |
| KLK10 | SKAP1 | WFDC2 |
| KLK10 | SPINK5 | TANK |
| KLK10 | SPINK5 | WFDC2 |
| KLK10 | TANK | WFDC2 |
| KRT19 | MUC-16 | PTK7 |
| KRT19 | MUC-16 | SEZ6L2 |
| KRT19 | MUC-16 | SPINT1 |
| KRT19 | MUC-16 | TACSTD2 |
| KRT19 | MUC-16 | CD83 |
| KRT19 | MUC-16 | CD99L2 |
| KRT19 | MUC-16 | CLEC4D |
| KRT19 | MUC-16 | CLEC6A |
| KRT19 | MUC-16 | CLMP |
| KRT19 | MUC-16 | CPE |
| KRT19 | MUC-16 | FR-alpha |
| KRT19 | MUC-16 | ICOSLG |
| KRT19 | MUC-16 | IRF9 |
| KRT19 | MUC-16 | LAMP3 |
| KRT19 | MUC-16 | MSMB |
| KRT19 | MUC-16 | PROK1 |
| KRT19 | MUC-16 | SIT1 |
| KRT19 | MUC-16 | SKAP1 |
| KRT19 | MUC-16 | SPINK5 |
| KRT19 | MUC-16 | TANK |
| KRT19 | MUC-16 | WFDC2 |
| KRT19 | PTK7 | SEZ6L2 |
| KRT19 | PTK7 | SPINT1 |
| KRT19 | PTK7 | TACSTD2 |
| KRT19 | PTK7 | CD83 |
| KRT19 | PTK7 | CD99L2 |
| KRT19 | PTK7 | CLEC4D |
| KRT19 | PTK7 | CLEC6A |
| KRT19 | PTK7 | CLMP |
| KRT19 | PTK7 | CPE |
| KRT19 | PTK7 | FR-alpha |
| KRT19 | PTK7 | ICOSLG |
| KRT19 | PTK7 | IRF9 |
| KRT19 | PTK7 | LAMP3 |
| KRT19 | PTK7 | MSMB |
| KRT19 | PTK7 | PROK1 |
| KRT19 | PTK7 | SIT1 |
| KRT19 | PTK7 | SKAP1 |
| KRT19 | PTK7 | SPINK5 |
| KRT19 | PTK7 | TANK |
| KRT19 | PTK7 | WFDC2 |
| KRT19 | SEZ6L2 | SPINT1 |
| KRT19 | SEZ6L2 | TACSTD2 |
| KRT19 | SEZ6L2 | CD83 |
| KRT19 | SEZ6L2 | CD99L2 |
| KRT19 | SEZ6L2 | CLEC4D |
| KRT19 | SEZ6L2 | CLEC6A |
| KRT19 | SEZ6L2 | CLMP |
| KRT19 | SEZ6L2 | CPE |
| KRT19 | SEZ6L2 | FR-alpha |
| KRT19 | SEZ6L2 | ICOSLG |
| KRT19 | SEZ6L2 | IRF9 |
| KRT19 | SEZ6L2 | LAMP3 |
| KRT19 | SEZ6L2 | MSMB |
| KRT19 | SEZ6L2 | PROK1 |
| KRT19 | SEZ6L2 | SIT1 |
| KRT19 | SEZ6L2 | SKAP1 |
| KRT19 | SEZ6L2 | SPINK5 |
| KRT19 | SEZ6L2 | TANK |
| KRT19 | SEZ6L2 | WFDC2 |
| KRT19 | SPINT1 | TACSTD2 |
| KRT19 | SPINT1 | CD83 |
| KRT19 | SPINT1 | CD99L2 |
| KRT19 | SPINT1 | CLEC4D |
| KRT19 | SPINT1 | CLEC6A |
| KRT19 | SPINT1 | CLMP |
| KRT19 | SPINT1 | CPE |
| KRT19 | SPINT1 | FR-alpha |
| KRT19 | SPINT1 | ICOSLG |
| KRT19 | SPINT1 | IRF9 |
| KRT19 | SPINT1 | LAMP3 |
| KRT19 | SPINT1 | MSMB |
| KRT19 | SPINT1 | PROK1 |
| KRT19 | SPINT1 | SIT1 |
| KRT19 | SPINT1 | SKAP1 |
| KRT19 | SPINT1 | SPINK5 |
| KRT19 | SPINT1 | TANK |
| KRT19 | SPINT1 | WFDC2 |
| KRT19 | TACSTD2 | CD83 |
| KRT19 | TACSTD2 | CD99L2 |
| KRT19 | TACSTD2 | CLEC4D |
| KRT19 | TACSTD2 | CLEC6A |
| KRT19 | TACSTD2 | CLMP |
| KRT19 | TACSTD2 | CPE |
| KRT19 | TACSTD2 | FR-alpha |
| KRT19 | TACSTD2 | ICOSLG |
| KRT19 | TACSTD2 | IRF9 |
| KRT19 | TACSTD2 | LAMP3 |
| KRT19 | TACSTD2 | MSMB |
| KRT19 | TACSTD2 | PROK1 |
| KRT19 | TACSTD2 | SIT1 |
| KRT19 | TACSTD2 | SKAP1 |
| KRT19 | TACSTD2 | SPINK5 |
| KRT19 | TACSTD2 | TANK |
| KRT19 | TACSTD2 | WFDC2 |
| KRT19 | CD83 | CD99L2 |
| KRT19 | CD83 | CLEC4D |
| KRT19 | CD83 | CLEC6A |
| KRT19 | CD83 | CLMP |
| KRT19 | CD83 | CPE |
| KRT19 | CD83 | FR-alpha |
| KRT19 | CD83 | ICOSLG |
| KRT19 | CD83 | IRF9 |
| KRT19 | CD83 | LAMP3 |
| KRT19 | CD83 | MSMB |
| KRT19 | CD83 | PROK1 |
| KRT19 | CD83 | SIT1 |
| KRT19 | CD83 | SKAP1 |
| KRT19 | CD83 | SPINK5 |
| KRT19 | CD83 | TANK |
| KRT19 | CD83 | WFDC2 |
| KRT19 | CD99L2 | CLEC4D |
| KRT19 | CD99L2 | CLEC6A |
| KRT19 | CD99L2 | CLMP |
| KRT19 | CD99L2 | CPE |
| KRT19 | CD99L2 | FR-alpha |
| KRT19 | CD99L2 | ICOSLG |
| KRT19 | CD99L2 | IRF9 |
| KRT19 | CD99L2 | LAMP3 |
| KRT19 | CD99L2 | MSMB |
| KRT19 | CD99L2 | PROK1 |
| KRT19 | CD99L2 | SIT1 |
| KRT19 | CD99L2 | SKAP1 |
| KRT19 | CD99L2 | SPINK5 |
| KRT19 | CD99L2 | TANK |
| KRT19 | CD99L2 | WFDC2 |
| KRT19 | CLEC4D | CLEC6A |
| KRT19 | CLEC4D | CLMP |
| KRT19 | CLEC4D | CPE |
| KRT19 | CLEC4D | FR-alpha |
| KRT19 | CLEC4D | ICOSLG |
| KRT19 | CLEC4D | IRF9 |
| KRT19 | CLEC4D | LAMP3 |
| KRT19 | CLEC4D | MSMB |
| KRT19 | CLEC4D | PROK1 |
| KRT19 | CLEC4D | SIT1 |
| KRT19 | CLEC4D | SKAP1 |
| KRT19 | CLEC4D | SPINK5 |
| KRT19 | CLEC4D | TANK |
| KRT19 | CLEC4D | WFDC2 |
| KRT19 | CLEC6A | CLMP |
| KRT19 | CLEC6A | CPE |
| KRT19 | CLEC6A | FR-alpha |
| KRT19 | CLEC6A | ICOSLG |
| KRT19 | CLEC6A | IRF9 |
| KRT19 | CLEC6A | LAMP3 |
| KRT19 | CLEC6A | MSMB |
| KRT19 | CLEC6A | PROK1 |
| KRT19 | CLEC6A | SIT1 |
| KRT19 | CLEC6A | SKAP1 |
| KRT19 | CLEC6A | SPINK5 |
| KRT19 | CLEC6A | TANK |
| KRT19 | CLEC6A | WFDC2 |
| KRT19 | CLMP | CPE |
| KRT19 | CLMP | FR-alpha |
| KRT19 | CLMP | ICOSLG |
| KRT19 | CLMP | IRF9 |
| KRT19 | CLMP | LAMP3 |
| KRT19 | CLMP | MSMB |
| KRT19 | CLMP | PROK1 |
| KRT19 | CLMP | SIT1 |
| KRT19 | CLMP | SKAP1 |
| KRT19 | CLMP | SPINK5 |
| KRT19 | CLMP | TANK |
| KRT19 | CLMP | WFDC2 |
| KRT19 | CPE | FR-alpha |
| KRT19 | CPE | ICOSLG |
| KRT19 | CPE | IRF9 |
| KRT19 | CPE | LAMP3 |
| KRT19 | CPE | MSMB |
| KRT19 | CPE | PROK1 |
| KRT19 | CPE | SIT1 |
| KRT19 | CPE | SKAP1 |
| KRT19 | CPE | SPINK5 |
| KRT19 | CPE | TANK |
| KRT19 | CPE | WFDC2 |
| KRT19 | FR-alpha | ICOSLG |
| KRT19 | FR-alpha | IRF9 |
| KRT19 | FR-alpha | LAMP3 |
| KRT19 | FR-alpha | MSMB |
| KRT19 | FR-alpha | PROK1 |
| KRT19 | FR-alpha | SIT1 |
| KRT19 | FR-alpha | SKAP1 |
| KRT19 | FR-alpha | SPINK5 |
| KRT19 | FR-alpha | TANK |
| KRT19 | FR-alpha | WFDC2 |
| KRT19 | ICOSLG | IRF9 |
| KRT19 | ICOSLG | LAMP3 |
| KRT19 | ICOSLG | MSMB |
| KRT19 | ICOSLG | PROK1 |
| KRT19 | ICOSLG | SIT1 |
| KRT19 | ICOSLG | SKAP1 |
| KRT19 | ICOSLG | SPINK5 |
| KRT19 | ICOSLG | TANK |
| KRT19 | ICOSLG | WFDC2 |
| KRT19 | IRF9 | LAMP3 |
| KRT19 | IRF9 | MSMB |
| KRT19 | IRF9 | PROK1 |
| KRT19 | IRF9 | SIT1 |
| KRT19 | IRF9 | SKAP1 |
| KRT19 | IRF9 | SPINK5 |
| KRT19 | IRF9 | TANK |
| KRT19 | IRF9 | WFDC2 |
| KRT19 | LAMP3 | MSMB |
| KRT19 | LAMP3 | PROK1 |
| KRT19 | LAMP3 | SIT1 |
| KRT19 | LAMP3 | SKAP1 |
| KRT19 | LAMP3 | SPINK5 |
| KRT19 | LAMP3 | TANK |
| KRT19 | LAMP3 | WFDC2 |
| KRT19 | MSMB | PROK1 |
| KRT19 | MSMB | SIT1 |
| KRT19 | MSMB | SKAP1 |
| KRT19 | MSMB | SPINK5 |
| KRT19 | MSMB | TANK |
| KRT19 | MSMB | WFDC2 |
| KRT19 | PROK1 | SIT1 |
| KRT19 | PROK1 | SKAP1 |
| KRT19 | PROK1 | SPINK5 |
| KRT19 | PROK1 | TANK |
| KRT19 | PROK1 | WFDC2 |
| KRT19 | SIT1 | SKAP1 |
| KRT19 | SIT1 | SPINK5 |
| KRT19 | SIT1 | TANK |
| KRT19 | SIT1 | WFDC2 |
| KRT19 | SKAP1 | SPINK5 |
| KRT19 | SKAP1 | TANK |
| KRT19 | SKAP1 | WFDC2 |
| KRT19 | SPINK5 | TANK |
| KRT19 | SPINK5 | WFDC2 |
| KRT19 | TANK | WFDC2 |
| MUC-16 | PTK7 | SEZ6L2 |
| MUC-16 | PTK7 | SPINT1 |
| MUC-16 | PTK7 | TACSTD2 |
| MUC-16 | PTK7 | CD83 |
| MUC-16 | PTK7 | CD99L2 |
| MUC-16 | PTK7 | CLEC4D |
| MUC-16 | PTK7 | CLEC6A |
| MUC-16 | PTK7 | CLMP |
| MUC-16 | PTK7 | CPE |
| MUC-16 | PTK7 | FR-alpha |
| MUC-16 | PTK7 | ICOSLG |
| MUC-16 | PTK7 | IRF9 |
| MUC-16 | PTK7 | LAMP3 |
| MUC-16 | PTK7 | MSMB |
| MUC-16 | PTK7 | PROK1 |
| MUC-16 | PTK7 | SIT1 |
| MUC-16 | PTK7 | SKAP1 |
| MUC-16 | PTK7 | SPINK5 |
| MUC-16 | PTK7 | TANK |
| MUC-16 | PTK7 | WFDC2 |
| MUC-16 | SEZ6L2 | SPINT1 |
| MUC-16 | SEZ6L2 | TACSTD2 |
| MUC-16 | SEZ6L2 | CD83 |
| MUC-16 | SEZ6L2 | CD99L2 |
| MUC-16 | SEZ6L2 | CLEC4D |
| MUC-16 | SEZ6L2 | CLEC6A |
| MUC-16 | SEZ6L2 | CLMP |
| MUC-16 | SEZ6L2 | CPE |
| MUC-16 | SEZ6L2 | FR-alpha |
| MUC-16 | SEZ6L2 | ICOSLG |
| MUC-16 | SEZ6L2 | IRF9 |
| MUC-16 | SEZ6L2 | LAMP3 |
| MUC-16 | SEZ6L2 | MSMB |
| MUC-16 | SEZ6L2 | PROK1 |
| MUC-16 | SEZ6L2 | SIT1 |
| MUC-16 | SEZ6L2 | SKAP1 |
| MUC-16 | SEZ6L2 | SPINK5 |
| MUC-16 | SEZ6L2 | TANK |
| MUC-16 | SEZ6L2 | WFDC2 |
| MUC-16 | SPINT1 | TACSTD2 |
| MUC-16 | SPINT1 | CD83 |
| MUC-16 | SPINT1 | CD99L2 |
| MUC-16 | SPINT1 | CLEC4D |
| MUC-16 | SPINT1 | CLEC6A |
| MUC-16 | SPINT1 | CLMP |
| MUC-16 | SPINT1 | CPE |
| MUC-16 | SPINT1 | FR-alpha |
| MUC-16 | SPINT1 | ICOSLG |
| MUC-16 | SPINT1 | IRF9 |
| MUC-16 | SPINT1 | LAMP3 |
| MUC-16 | SPINT1 | MSMB |
| MUC-16 | SPINT1 | PROK1 |
| MUC-16 | SPINT1 | SIT1 |
| MUC-16 | SPINT1 | SKAP1 |
| MUC-16 | SPINT1 | SPINK5 |
| MUC-16 | SPINT1 | TANK |
| MUC-16 | SPINT1 | WFDC2 |
| MUC-16 | TACSTD2 | CD83 |
| MUC-16 | TACSTD2 | CD99L2 |
| MUC-16 | TACSTD2 | CLEC4D |
| MUC-16 | TACSTD2 | CLEC6A |
| MUC-16 | TACSTD2 | CLMP |
| MUC-16 | TACSTD2 | CPE |
| MUC-16 | TACSTD2 | FR-alpha |
| MUC-16 | TACSTD2 | ICOSLG |
| MUC-16 | TACSTD2 | IRF9 |
| MUC-16 | TACSTD2 | LAMP3 |
| MUC-16 | TACSTD2 | MSMB |
| MUC-16 | TACSTD2 | PROK1 |
| MUC-16 | TACSTD2 | SIT1 |
| MUC-16 | TACSTD2 | SKAP1 |
| MUC-16 | TACSTD2 | SPINK5 |
| MUC-16 | TACSTD2 | TANK |
| MUC-16 | TACSTD2 | WFDC2 |
| MUC-16 | CD83 | CD99L2 |
| MUC-16 | CD83 | CLEC4D |
| MUC-16 | CD83 | CLEC6A |
| MUC-16 | CD83 | CLMP |
| MUC-16 | CD83 | CPE |
| MUC-16 | CD83 | FR-alpha |
| MUC-16 | CD83 | ICOSLG |
| MUC-16 | CD83 | IRF9 |
| MUC-16 | CD83 | LAMP3 |
| MUC-16 | CD83 | MSMB |
| MUC-16 | CD83 | PROK1 |
| MUC-16 | CD83 | SIT1 |
| MUC-16 | CD83 | SKAP1 |
| MUC-16 | CD83 | SPINK5 |
| MUC-16 | CD83 | TANK |
| MUC-16 | CD83 | WFDC2 |
| MUC-16 | CD99L2 | CLEC4D |
| MUC-16 | CD99L2 | CLEC6A |
| MUC-16 | CD99L2 | CLMP |
| MUC-16 | CD99L2 | CPE |
| MUC-16 | CD99L2 | FR-alpha |
| MUC-16 | CD99L2 | ICOSLG |
| MUC-16 | CD99L2 | IRF9 |
| MUC-16 | CD99L2 | LAMP3 |
| MUC-16 | CD99L2 | MSMB |
| MUC-16 | CD99L2 | PROK1 |
| MUC-16 | CD99L2 | SIT1 |
| MUC-16 | CD99L2 | SKAP1 |
| MUC-16 | CD99L2 | SPINK5 |
| MUC-16 | CD99L2 | TANK |
| MUC-16 | CD99L2 | WFDC2 |
| MUC-16 | CLEC4D | CLEC6A |
| MUC-16 | CLEC4D | CLMP |
| MUC-16 | CLEC4D | CPE |
| MUC-16 | CLEC4D | FR-alpha |
| MUC-16 | CLEC4D | ICOSLG |
| MUC-16 | CLEC4D | IRF9 |
| MUC-16 | CLEC4D | LAMP3 |
| MUC-16 | CLEC4D | MSMB |
| MUC-16 | CLEC4D | PROK1 |
| MUC-16 | CLEC4D | SIT1 |
| MUC-16 | CLEC4D | SKAP1 |
| MUC-16 | CLEC4D | SPINK5 |
| MUC-16 | CLEC4D | TANK |
| MUC-16 | CLEC4D | WFDC2 |
| MUC-16 | CLEC6A | CLMP |
| MUC-16 | CLEC6A | CPE |
| MUC-16 | CLEC6A | FR-alpha |
| MUC-16 | CLEC6A | ICOSLG |
| MUC-16 | CLEC6A | IRF9 |
| MUC-16 | CLEC6A | LAMP3 |
| MUC-16 | CLEC6A | MSMB |
| MUC-16 | CLEC6A | PROK1 |
| MUC-16 | CLEC6A | SIT1 |
| MUC-16 | CLEC6A | SKAP1 |
| MUC-16 | CLEC6A | SPINK5 |
| MUC-16 | CLEC6A | TANK |
| MUC-16 | CLEC6A | WFDC2 |
| MUC-16 | CLMP | CPE |
| MUC-16 | CLMP | FR-alpha |
| MUC-16 | CLMP | ICOSLG |
| MUC-16 | CLMP | IRF9 |
| MUC-16 | CLMP | LAMP3 |
| MUC-16 | CLMP | MSMB |
| MUC-16 | CLMP | PROK1 |
| MUC-16 | CLMP | SIT1 |
| MUC-16 | CLMP | SKAP1 |
| MUC-16 | CLMP | SPINK5 |
| MUC-16 | CLMP | TANK |
| MUC-16 | CLMP | WFDC2 |
| MUC-16 | CPE | FR-alpha |
| MUC-16 | CPE | ICOSLG |
| MUC-16 | CPE | IRF9 |
| MUC-16 | CPE | LAMP3 |
| MUC-16 | CPE | MSMB |
| MUC-16 | CPE | PROK1 |
| MUC-16 | CPE | SIT1 |
| MUC-16 | CPE | SKAP1 |
| MUC-16 | CPE | SPINK5 |
| MUC-16 | CPE | TANK |
| MUC-16 | CPE | WFDC2 |
| MUC-16 | FR-alpha | ICOSLG |
| MUC-16 | FR-alpha | IRF9 |
| MUC-16 | FR-alpha | LAMP3 |
| MUC-16 | FR-alpha | MSMB |
| MUC-16 | FR-alpha | PROK1 |
| MUC-16 | FR-alpha | SIT1 |
| MUC-16 | FR-alpha | SKAP1 |
| MUC-16 | FR-alpha | SPINK5 |
| MUC-16 | FR-alpha | TANK |
| MUC-16 | FR-alpha | WFDC2 |
| MUC-16 | ICOSLG | IRF9 |
| MUC-16 | ICOSLG | LAMP3 |
| MUC-16 | ICOSLG | MSMB |
| MUC-16 | ICOSLG | PROK1 |
| MUC-16 | ICOSLG | SIT1 |
| MUC-16 | ICOSLG | SKAP1 |
| MUC-16 | ICOSLG | SPINK5 |
| MUC-16 | ICOSLG | TANK |
| MUC-16 | ICOSLG | WFDC2 |
| MUC-16 | IRF9 | LAMP3 |
| MUC-16 | IRF9 | MSMB |
| MUC-16 | IRF9 | PROK1 |
| MUC-16 | IRF9 | SIT1 |
| MUC-16 | IRF9 | SKAP1 |
| MUC-16 | IRF9 | SPINK5 |
| MUC-16 | IRF9 | TANK |
| MUC-16 | IRF9 | WFDC2 |
| MUC-16 | LAMP3 | MSMB |
| MUC-16 | LAMP3 | PROK1 |
| MUC-16 | LAMP3 | SIT1 |
| MUC-16 | LAMP3 | SKAP1 |
| MUC-16 | LAMP3 | SPINK5 |
| MUC-16 | LAMP3 | TANK |
| MUC-16 | LAMP3 | WFDC2 |
| MUC-16 | MSMB | PROK1 |
| MUC-16 | MSMB | SIT1 |
| MUC-16 | MSMB | SKAP1 |
| MUC-16 | MSMB | SPINK5 |
| MUC-16 | MSMB | TANK |
| MUC-16 | MSMB | WFDC2 |
| MUC-16 | PROK1 | SIT1 |
| MUC-16 | PROK1 | SKAP1 |
| MUC-16 | PROK1 | SPINK5 |
| MUC-16 | PROK1 | TANK |
| MUC-16 | PROK1 | WFDC2 |
| MUC-16 | SIT1 | SKAP1 |
| MUC-16 | SIT1 | SPINK5 |
| MUC-16 | SIT1 | TANK |
| MUC-16 | SIT1 | WFDC2 |
| MUC-16 | SKAP1 | SPINK5 |
| MUC-16 | SKAP1 | TANK |
| MUC-16 | SKAP1 | WFDC2 |
| MUC-16 | SPINK5 | TANK |
| MUC-16 | SPINK5 | WFDC2 |
| MUC-16 | TANK | WFDC2 |
| PTK7 | SEZ6L2 | SPINT1 |
| PTK7 | SEZ6L2 | TACSTD2 |
| PTK7 | SEZ6L2 | CD83 |
| PTK7 | SEZ6L2 | CD99L2 |
| PTK7 | SEZ6L2 | CLEC4D |
| PTK7 | SEZ6L2 | CLEC6A |
| PTK7 | SEZ6L2 | CLMP |
| PTK7 | SEZ6L2 | CPE |
| PTK7 | SEZ6L2 | FR-alpha |
| PTK7 | SEZ6L2 | ICOSLG |
| PTK7 | SEZ6L2 | IRF9 |
| PTK7 | SEZ6L2 | LAMP3 |
| PTK7 | SEZ6L2 | MSMB |
| PTK7 | SEZ6L2 | PROK1 |
| PTK7 | SEZ6L2 | SIT1 |
| PTK7 | SEZ6L2 | SKAP1 |
| PTK7 | SEZ6L2 | SPINK5 |
| PTK7 | SEZ6L2 | TANK |
| PTK7 | SEZ6L2 | WFDC2 |
| PTK7 | SPINT1 | TACSTD2 |
| PTK7 | SPINT1 | CD83 |
| PTK7 | SPINT1 | CD99L2 |
| PTK7 | SPINT1 | CLEC4D |
| PTK7 | SPINT1 | CLEC6A |
| PTK7 | SPINT1 | CLMP |
| PTK7 | SPINT1 | CPE |
| PTK7 | SPINT1 | FR-alpha |
| PTK7 | SPINT1 | ICOSLG |
| PTK7 | SPINT1 | IRF9 |
| PTK7 | SPINT1 | LAMP3 |
| PTK7 | SPINT1 | MSMB |
| PTK7 | SPINT1 | PROK1 |
| PTK7 | SPINT1 | SIT1 |
| PTK7 | SPINT1 | SKAP1 |
| PTK7 | SPINT1 | SPINK5 |
| PTK7 | SPINT1 | TANK |
| PTK7 | SPINT1 | WFDC2 |
| PTK7 | TACSTD2 | CD83 |
| PTK7 | TACSTD2 | CD99L2 |
| PTK7 | TACSTD2 | CLEC4D |
| PTK7 | TACSTD2 | CLEC6A |
| PTK7 | TACSTD2 | CLMP |
| PTK7 | TACSTD2 | CPE |
| PTK7 | TACSTD2 | FR-alpha |
| PTK7 | TACSTD2 | ICOSLG |
| PTK7 | TACSTD2 | IRF9 |
| PTK7 | TACSTD2 | LAMP3 |
| PTK7 | TACSTD2 | MSMB |
| PTK7 | TACSTD2 | PROK1 |
| PTK7 | TACSTD2 | SIT1 |
| PTK7 | TACSTD2 | SKAP1 |
| PTK7 | TACSTD2 | SPINK5 |
| PTK7 | TACSTD2 | TANK |
| PTK7 | TACSTD2 | WFDC2 |
| PTK7 | CD83 | CD99L2 |
| PTK7 | CD83 | CLEC4D |
| PTK7 | CD83 | CLEC6A |
| PTK7 | CD83 | CLMP |
| PTK7 | CD83 | CPE |
| PTK7 | CD83 | FR-alpha |
| PTK7 | CD83 | ICOSLG |
| PTK7 | CD83 | IRF9 |
| PTK7 | CD83 | LAMP3 |
| PTK7 | CD83 | MSMB |
| PTK7 | CD83 | PROK1 |
| PTK7 | CD83 | SIT1 |
| PTK7 | CD83 | SKAP1 |
| PTK7 | CD83 | SPINK5 |
| PTK7 | CD83 | TANK |
| PTK7 | CD83 | WFDC2 |
| PTK7 | CD99L2 | CLEC4D |
| PTK7 | CD99L2 | CLEC6A |
| PTK7 | CD99L2 | CLMP |
| PTK7 | CD99L2 | CPE |
| PTK7 | CD99L2 | FR-alpha |
| PTK7 | CD99L2 | ICOSLG |
| PTK7 | CD99L2 | IRF9 |
| PTK7 | CD99L2 | LAMP3 |
| PTK7 | CD99L2 | MSMB |
| PTK7 | CD99L2 | PROK1 |
| PTK7 | CD99L2 | SIT1 |
| PTK7 | CD99L2 | SKAP1 |
| PTK7 | CD99L2 | SPINK5 |
| PTK7 | CD99L2 | TANK |
| PTK7 | CD99L2 | WFDC2 |
| PTK7 | CLEC4D | CLEC6A |
| PTK7 | CLEC4D | CLMP |
| PTK7 | CLEC4D | CPE |
| PTK7 | CLEC4D | FR-alpha |
| PTK7 | CLEC4D | ICOSLG |
| PTK7 | CLEC4D | IRF9 |
| PTK7 | CLEC4D | LAMP3 |
| PTK7 | CLEC4D | MSMB |
| PTK7 | CLEC4D | PROK1 |
| PTK7 | CLEC4D | SIT1 |
| PTK7 | CLEC4D | SKAP1 |
| PTK7 | CLEC4D | SPINK5 |
| PTK7 | CLEC4D | TANK |
| PTK7 | CLEC4D | WFDC2 |
| PTK7 | CLEC6A | CLMP |
| PTK7 | CLEC6A | CPE |
| PTK7 | CLEC6A | FR-alpha |
| PTK7 | CLEC6A | ICOSLG |
| PTK7 | CLEC6A | IRF9 |
| PTK7 | CLEC6A | LAMP3 |
| PTK7 | CLEC6A | MSMB |
| PTK7 | CLEC6A | PROK1 |
| PTK7 | CLEC6A | SIT1 |
| PTK7 | CLEC6A | SKAP1 |
| PTK7 | CLEC6A | SPINK5 |
| PTK7 | CLEC6A | TANK |
| PTK7 | CLEC6A | WFDC2 |
| PTK7 | CLMP | CPE |
| PTK7 | CLMP | FR-alpha |
| PTK7 | CLMP | ICOSLG |
| PTK7 | CLMP | IRF9 |
| PTK7 | CLMP | LAMP3 |
| PTK7 | CLMP | MSMB |
| PTK7 | CLMP | PROK1 |
| PTK7 | CLMP | SIT1 |
| PTK7 | CLMP | SKAP1 |
| PTK7 | CLMP | SPINK5 |
| PTK7 | CLMP | TANK |
| PTK7 | CLMP | WFDC2 |
| PTK7 | CPE | FR-alpha |
| PTK7 | CPE | ICOSLG |
| PTK7 | CPE | IRF9 |
| PTK7 | CPE | LAMP3 |
| PTK7 | CPE | MSMB |
| PTK7 | CPE | PROK1 |
| PTK7 | CPE | SIT1 |
| PTK7 | CPE | SKAP1 |
| PTK7 | CPE | SPINK5 |
| PTK7 | CPE | TANK |
| PTK7 | CPE | WFDC2 |
| PTK7 | FR-alpha | ICOSLG |
| PTK7 | FR-alpha | IRF9 |
| PTK7 | FR-alpha | LAMP3 |
| PTK7 | FR-alpha | MSMB |
| PTK7 | FR-alpha | PROK1 |
| PTK7 | FR-alpha | SIT1 |
| PTK7 | FR-alpha | SKAP1 |
| PTK7 | FR-alpha | SPINK5 |
| PTK7 | FR-alpha | TANK |
| PTK7 | FR-alpha | WFDC2 |
| PTK7 | ICOSLG | IRF9 |
| PTK7 | ICOSLG | LAMP3 |
| PTK7 | ICOSLG | MSMB |
| PTK7 | ICOSLG | PROK1 |
| PTK7 | ICOSLG | SIT1 |
| PTK7 | ICOSLG | SKAP1 |
| PTK7 | ICOSLG | SPINK5 |
| PTK7 | ICOSLG | TANK |
| PTK7 | ICOSLG | WFDC2 |
| PTK7 | IRF9 | LAMP3 |
| PTK7 | IRF9 | MSMB |
| PTK7 | IRF9 | PROK1 |
| PTK7 | IRF9 | SIT1 |
| PTK7 | IRF9 | SKAP1 |
| PTK7 | IRF9 | SPINK5 |
| PTK7 | IRF9 | TANK |
| PTK7 | IRF9 | WFDC2 |
| PTK7 | LAMP3 | MSMB |
| PTK7 | LAMP3 | PROK1 |
| PTK7 | LAMP3 | SIT1 |
| PTK7 | LAMP3 | SKAP1 |
| PTK7 | LAMP3 | SPINK5 |
| PTK7 | LAMP3 | TANK |
| PTK7 | LAMP3 | WFDC2 |
| PTK7 | MSMB | PROK1 |
| PTK7 | MSMB | SIT1 |
| PTK7 | MSMB | SKAP1 |
| PTK7 | MSMB | SPINK5 |
| PTK7 | MSMB | TANK |
| PTK7 | MSMB | WFDC2 |
| PTK7 | PROK1 | SIT1 |
| PTK7 | PROK1 | SKAP1 |
| PTK7 | PROK1 | SPINK5 |
| PTK7 | PROK1 | TANK |
| PTK7 | PROK1 | WFDC2 |
| PTK7 | SIT1 | SKAP1 |
| PTK7 | SIT1 | SPINK5 |
| PTK7 | SIT1 | TANK |
| PTK7 | SIT1 | WFDC2 |
| PTK7 | SKAP1 | SPINK5 |
| PTK7 | SKAP1 | TANK |
| PTK7 | SKAP1 | WFDC2 |
| PTK7 | SPINK5 | TANK |
| PTK7 | SPINK5 | WFDC2 |
| PTK7 | TANK | WFDC2 |
| SEZ6L2 | SPINT1 | TACSTD2 |
| SEZ6L2 | SPINT1 | CD83 |
| SEZ6L2 | SPINT1 | CD99L2 |
| SEZ6L2 | SPINT1 | CLEC4D |
| SEZ6L2 | SPINT1 | CLEC6A |
| SEZ6L2 | SPINT1 | CLMP |
| SEZ6L2 | SPINT1 | CPE |
| SEZ6L2 | SPINT1 | FR-alpha |
| SEZ6L2 | SPINT1 | ICOSLG |
| SEZ6L2 | SPINT1 | IRF9 |
| SEZ6L2 | SPINT1 | LAMP3 |
| SEZ6L2 | SPINT1 | MSMB |
| SEZ6L2 | SPINT1 | PROK1 |
| SEZ6L2 | SPINT1 | SIT1 |
| SEZ6L2 | SPINT1 | SKAP1 |
| SEZ6L2 | SPINT1 | SPINK5 |
| SEZ6L2 | SPINT1 | TANK |
| SEZ6L2 | SPINT1 | WFDC2 |
| SEZ6L2 | TACSTD2 | CD83 |
| SEZ6L2 | TACSTD2 | CD99L2 |
| SEZ6L2 | TACSTD2 | CLEC4D |
| SEZ6L2 | TACSTD2 | CLEC6A |
| SEZ6L2 | TACSTD2 | CLMP |
| SEZ6L2 | TACSTD2 | CPE |
| SEZ6L2 | TACSTD2 | FR-alpha |
| SEZ6L2 | TACSTD2 | ICOSLG |
| SEZ6L2 | TACSTD2 | IRF9 |
| SEZ6L2 | TACSTD2 | LAMP3 |
| SEZ6L2 | TACSTD2 | MSMB |
| SEZ6L2 | TACSTD2 | PROK1 |
| SEZ6L2 | TACSTD2 | SIT1 |
| SEZ6L2 | TACSTD2 | SKAP1 |
| SEZ6L2 | TACSTD2 | SPINK5 |
| SEZ6L2 | TACSTD2 | TANK |
| SEZ6L2 | TACSTD2 | WFDC2 |
| SEZ6L2 | CD83 | CD99L2 |
| SEZ6L2 | CD83 | CLEC4D |
| SEZ6L2 | CD83 | CLEC6A |
| SEZ6L2 | CD83 | CLMP |
| SEZ6L2 | CD83 | CPE |
| SEZ6L2 | CD83 | FR-alpha |
| SEZ6L2 | CD83 | ICOSLG |
| SEZ6L2 | CD83 | IRF9 |
| SEZ6L2 | CD83 | LAMP3 |
| SEZ6L2 | CD83 | MSMB |
| SEZ6L2 | CD83 | PROK1 |
| SEZ6L2 | CD83 | SIT1 |
| SEZ6L2 | CD83 | SKAP1 |
| SEZ6L2 | CD83 | SPINK5 |
| SEZ6L2 | CD83 | TANK |
| SEZ6L2 | CD83 | WFDC2 |
| SEZ6L2 | CD99L2 | CLEC4D |
| SEZ6L2 | CD99L2 | CLEC6A |
| SEZ6L2 | CD99L2 | CLMP |
| SEZ6L2 | CD99L2 | CPE |
| SEZ6L2 | CD99L2 | FR-alpha |
| SEZ6L2 | CD99L2 | ICOSLG |
| SEZ6L2 | CD99L2 | IRF9 |
| SEZ6L2 | CD99L2 | LAMP3 |
| SEZ6L2 | CD99L2 | MSMB |
| SEZ6L2 | CD99L2 | PROK1 |
| SEZ6L2 | CD99L2 | SIT1 |
| SEZ6L2 | CD99L2 | SKAP1 |
| SEZ6L2 | CD99L2 | SPINK5 |
| SEZ6L2 | CD99L2 | TANK |
| SEZ6L2 | CD99L2 | WFDC2 |
| SEZ6L2 | CLEC4D | CLEC6A |
| SEZ6L2 | CLEC4D | CLMP |
| SEZ6L2 | CLEC4D | CPE |
| SEZ6L2 | CLEC4D | FR-alpha |
| SEZ6L2 | CLEC4D | ICOSLG |
| SEZ6L2 | CLEC4D | IRF9 |
| SEZ6L2 | CLEC4D | LAMP3 |
| SEZ6L2 | CLEC4D | MSMB |
| SEZ6L2 | CLEC4D | PROK1 |
| SEZ6L2 | CLEC4D | SIT1 |
| SEZ6L2 | CLEC4D | SKAP1 |
| SEZ6L2 | CLEC4D | SPINK5 |
| SEZ6L2 | CLEC4D | TANK |
| SEZ6L2 | CLEC4D | WFDC2 |
| SEZ6L2 | CLEC6A | CLMP |
| SEZ6L2 | CLEC6A | CPE |
| SEZ6L2 | CLEC6A | FR-alpha |
| SEZ6L2 | CLEC6A | ICOSLG |
| SEZ6L2 | CLEC6A | IRF9 |
| SEZ6L2 | CLEC6A | LAMP3 |
| SEZ6L2 | CLEC6A | MSMB |
| SEZ6L2 | CLEC6A | PROK1 |
| SEZ6L2 | CLEC6A | SIT1 |
| SEZ6L2 | CLEC6A | SKAP1 |
| SEZ6L2 | CLEC6A | SPINK5 |
| SEZ6L2 | CLEC6A | TANK |
| SEZ6L2 | CLEC6A | WFDC2 |
| SEZ6L2 | CLMP | CPE |
| SEZ6L2 | CLMP | FR-alpha |
| SEZ6L2 | CLMP | ICOSLG |
| SEZ6L2 | CLMP | IRF9 |
| SEZ6L2 | CLMP | LAMP3 |
| SEZ6L2 | CLMP | MSMB |
| SEZ6L2 | CLMP | PROK1 |
| SEZ6L2 | CLMP | SIT1 |
| SEZ6L2 | CLMP | SKAP1 |
| SEZ6L2 | CLMP | SPINK5 |
| SEZ6L2 | CLMP | TANK |
| SEZ6L2 | CLMP | WFDC2 |
| SEZ6L2 | CPE | FR-alpha |
| SEZ6L2 | CPE | ICOSLG |
| SEZ6L2 | CPE | IRF9 |
| SEZ6L2 | CPE | LAMP3 |
| SEZ6L2 | CPE | MSMB |
| SEZ6L2 | CPE | PROK1 |
| SEZ6L2 | CPE | SIT1 |
| SEZ6L2 | CPE | SKAP1 |
| SEZ6L2 | CPE | SPINK5 |
| SEZ6L2 | CPE | TANK |
| SEZ6L2 | CPE | WFDC2 |
| SEZ6L2 | FR-alpha | ICOSLG |
| SEZ6L2 | FR-alpha | IRF9 |
| SEZ6L2 | FR-alpha | LAMP3 |
| SEZ6L2 | FR-alpha | MSMB |
| SEZ6L2 | FR-alpha | PROK1 |
| SEZ6L2 | FR-alpha | SIT1 |
| SEZ6L2 | FR-alpha | SKAP1 |
| SEZ6L2 | FR-alpha | SPINK5 |
| SEZ6L2 | FR-alpha | TANK |
| SEZ6L2 | FR-alpha | WFDC2 |
| SEZ6L2 | ICOSLG | IRF9 |
| SEZ6L2 | ICOSLG | LAMP3 |
| SEZ6L2 | ICOSLG | MSMB |
| SEZ6L2 | ICOSLG | PROK1 |
| SEZ6L2 | ICOSLG | SIT1 |
| SEZ6L2 | ICOSLG | SKAP1 |
| SEZ6L2 | ICOSLG | SPINK5 |
| SEZ6L2 | ICOSLG | TANK |
| SEZ6L2 | ICOSLG | WFDC2 |
| SEZ6L2 | IRF9 | LAMP3 |
| SEZ6L2 | IRF9 | MSMB |
| SEZ6L2 | IRF9 | PROK1 |
| SEZ6L2 | IRF9 | SIT1 |
| SEZ6L2 | IRF9 | SKAP1 |
| SEZ6L2 | IRF9 | SPINK5 |
| SEZ6L2 | IRF9 | TANK |
| SEZ6L2 | IRF9 | WFDC2 |
| SEZ6L2 | LAMP3 | MSMB |
| SEZ6L2 | LAMP3 | PROK1 |
| SEZ6L2 | LAMP3 | SIT1 |
| SEZ6L2 | LAMP3 | SKAP1 |
| SEZ6L2 | LAMP3 | SPINK5 |
| SEZ6L2 | LAMP3 | TANK |
| SEZ6L2 | LAMP3 | WFDC2 |
| SEZ6L2 | MSMB | PROK1 |
| SEZ6L2 | MSMB | SIT1 |
| SEZ6L2 | MSMB | SKAP1 |
| SEZ6L2 | MSMB | SPINK5 |
| SEZ6L2 | MSMB | TANK |
| SEZ6L2 | MSMB | WFDC2 |
| SEZ6L2 | PROK1 | SIT1 |
| SEZ6L2 | PROK1 | SKAP1 |
| SEZ6L2 | PROK1 | SPINK5 |
| SEZ6L2 | PROK1 | TANK |
| SEZ6L2 | PROK1 | WFDC2 |
| SEZ6L2 | SIT1 | SKAP1 |
| SEZ6L2 | SIT1 | SPINK5 |
| SEZ6L2 | SIT1 | TANK |
| SEZ6L2 | SIT1 | WFDC2 |
| SEZ6L2 | SKAP1 | SPINK5 |
| SEZ6L2 | SKAP1 | TANK |
| SEZ6L2 | SKAP1 | WFDC2 |
| SEZ6L2 | SPINK5 | TANK |
| SEZ6L2 | SPINK5 | WFDC2 |
| SEZ6L2 | TANK | WFDC2 |
| SPINT1 | TACSTD2 | CD83 |
| SPINT1 | TACSTD2 | CD99L2 |
| SPINT1 | TACSTD2 | CLEC4D |
| SPINT1 | TACSTD2 | CLEC6A |
| SPINT1 | TACSTD2 | CLMP |
| SPINT1 | TACSTD2 | CPE |
| SPINT1 | TACSTD2 | FR-alpha |
| SPINT1 | TACSTD2 | ICOSLG |
| SPINT1 | TACSTD2 | IRF9 |
| SPINT1 | TACSTD2 | LAMP3 |
| SPINT1 | TACSTD2 | MSMB |
| SPINT1 | TACSTD2 | PROK1 |
| SPINT1 | TACSTD2 | SIT1 |
| SPINT1 | TACSTD2 | SKAP1 |
| SPINT1 | TACSTD2 | SPINK5 |
| SPINT1 | TACSTD2 | TANK |
| SPINT1 | TACSTD2 | WFDC2 |
| SPINT1 | CD83 | CD99L2 |
| SPINT1 | CD83 | CLEC4D |
| SPINT1 | CD83 | CLEC6A |
| SPINT1 | CD83 | CLMP |
| SPINT1 | CD83 | CPE |
| SPINT1 | CD83 | FR-alpha |
| SPINT1 | CD83 | ICOSLG |
| SPINT1 | CD83 | IRF9 |
| SPINT1 | CD83 | LAMP3 |
| SPINT1 | CD83 | MSMB |
| SPINT1 | CD83 | PROK1 |
| SPINT1 | CD83 | SIT1 |
| SPINT1 | CD83 | SKAP1 |
| SPINT1 | CD83 | SPINK5 |
| SPINT1 | CD83 | TANK |
| SPINT1 | CD83 | WFDC2 |
| SPINT1 | CD99L2 | CLEC4D |
| SPINT1 | CD99L2 | CLEC6A |
| SPINT1 | CD99L2 | CLMP |
| SPINT1 | CD99L2 | CPE |
| SPINT1 | CD99L2 | FR-alpha |
| SPINT1 | CD99L2 | ICOSLG |
| SPINT1 | CD99L2 | IRF9 |
| SPINT1 | CD99L2 | LAMP3 |
| SPINT1 | CD99L2 | MSMB |
| SPINT1 | CD99L2 | PROK1 |
| SPINT1 | CD99L2 | SIT1 |
| SPINT1 | CD99L2 | SKAP1 |
| SPINT1 | CD99L2 | SPINK5 |
| SPINT1 | CD99L2 | TANK |
| SPINT1 | CD99L2 | WFDC2 |
| SPINT1 | CLEC4D | CLEC6A |
| SPINT1 | CLEC4D | CLMP |
| SPINT1 | CLEC4D | CPE |
| SPINT1 | CLEC4D | FR-alpha |
| SPINT1 | CLEC4D | ICOSLG |
| SPINT1 | CLEC4D | IRF9 |
| SPINT1 | CLEC4D | LAMP3 |
| SPINT1 | CLEC4D | MSMB |
| SPINT1 | CLEC4D | PROK1 |
| SPINT1 | CLEC4D | SIT1 |
| SPINT1 | CLEC4D | SKAP1 |
| SPINT1 | CLEC4D | SPINK5 |
| SPINT1 | CLEC4D | TANK |
| SPINT1 | CLEC4D | WFDC2 |
| SPINT1 | CLEC6A | CLMP |
| SPINT1 | CLEC6A | CPE |
| SPINT1 | CLEC6A | FR-alpha |
| SPINT1 | CLEC6A | ICOSLG |
| SPINT1 | CLEC6A | IRF9 |
| SPINT1 | CLEC6A | LAMP3 |
| SPINT1 | CLEC6A | MSMB |
| SPINT1 | CLEC6A | PROK1 |
| SPINT1 | CLEC6A | SIT1 |
| SPINT1 | CLEC6A | SKAP1 |
| SPINT1 | CLEC6A | SPINK5 |
| SPINT1 | CLEC6A | TANK |
| SPINT1 | CLEC6A | WFDC2 |
| SPINT1 | CLMP | CPE |
| SPINT1 | CLMP | FR-alpha |
| SPINT1 | CLMP | ICOSLG |
| SPINT1 | CLMP | IRF9 |
| SPINT1 | CLMP | LAMP3 |
| SPINT1 | CLMP | MSMB |
| SPINT1 | CLMP | PROK1 |
| SPINT1 | CLMP | SIT1 |
| SPINT1 | CLMP | SKAP1 |
| SPINT1 | CLMP | SPINK5 |
| SPINT1 | CLMP | TANK |
| SPINT1 | CLMP | WFDC2 |
| SPINT1 | CPE | FR-alpha |
| SPINT1 | CPE | ICOSLG |
| SPINT1 | CPE | IRF9 |
| SPINT1 | CPE | LAMP3 |
| SPINT1 | CPE | MSMB |
| SPINT1 | CPE | PROK1 |
| SPINT1 | CPE | SIT1 |
| SPINT1 | CPE | SKAP1 |
| SPINT1 | CPE | SPINK5 |
| SPINT1 | CPE | TANK |
| SPINT1 | CPE | WFDC2 |
| SPINT1 | FR-alpha | ICOSLG |
| SPINT1 | FR-alpha | IRF9 |
| SPINT1 | FR-alpha | LAMP3 |
| SPINT1 | FR-alpha | MSMB |
| SPINT1 | FR-alpha | PROK1 |
| SPINT1 | FR-alpha | SIT1 |
| SPINT1 | FR-alpha | SKAP1 |
| SPINT1 | FR-alpha | SPINK5 |
| SPINT1 | FR-alpha | TANK |
| SPINT1 | FR-alpha | WFDC2 |
| SPINT1 | ICOSLG | IRF9 |
| SPINT1 | ICOSLG | LAMP3 |
| SPINT1 | ICOSLG | MSMB |
| SPINT1 | ICOSLG | PROK1 |
| SPINT1 | ICOSLG | SIT1 |
| SPINT1 | ICOSLG | SKAP1 |
| SPINT1 | ICOSLG | SPINK5 |
| SPINT1 | ICOSLG | TANK |
| SPINT1 | ICOSLG | WFDC2 |
| SPINT1 | IRF9 | LAMP3 |
| SPINT1 | IRF9 | MSMB |
| SPINT1 | IRF9 | PROK1 |
| SPINT1 | IRF9 | SIT1 |
| SPINT1 | IRF9 | SKAP1 |
| SPINT1 | IRF9 | SPINK5 |
| SPINT1 | IRF9 | TANK |
| SPINT1 | IRF9 | WFDC2 |
| SPINT1 | LAMP3 | MSMB |
| SPINT1 | LAMP3 | PROK1 |
| SPINT1 | LAMP3 | SIT1 |
| SPINT1 | LAMP3 | SKAP1 |
| SPINT1 | LAMP3 | SPINK5 |
| SPINT1 | LAMP3 | TANK |
| SPINT1 | LAMP3 | WFDC2 |
| SPINT1 | MSMB | PROK1 |
| SPINT1 | MSMB | SIT1 |
| SPINT1 | MSMB | SKAP1 |
| SPINT1 | MSMB | SPINK5 |
| SPINT1 | MSMB | TANK |
| SPINT1 | MSMB | WFDC2 |
| SPINT1 | PROK1 | SIT1 |
| SPINT1 | PROK1 | SKAP1 |
| SPINT1 | PROK1 | SPINK5 |
| SPINT1 | PROK1 | TANK |
| SPINT1 | PROK1 | WFDC2 |
| SPINT1 | SIT1 | SKAP1 |
| SPINT1 | SIT1 | SPINK5 |
| SPINT1 | SIT1 | TANK |
| SPINT1 | SIT1 | WFDC2 |
| SPINT1 | SKAP1 | SPINK5 |
| SPINT1 | SKAP1 | TANK |
| SPINT1 | SKAP1 | WFDC2 |
| SPINT1 | SPINK5 | TANK |
| SPINT1 | SPINK5 | WFDC2 |
| SPINT1 | TANK | WFDC2 |
| TACSTD2 | CD83 | CD99L2 |
| TACSTD2 | CD83 | CLEC4D |
| TACSTD2 | CD83 | CLEC6A |
| TACSTD2 | CD83 | CLMP |
| TACSTD2 | CD83 | CPE |
| TACSTD2 | CD83 | FR-alpha |
| TACSTD2 | CD83 | ICOSLG |
| TACSTD2 | CD83 | IRF9 |
| TACSTD2 | CD83 | LAMP3 |
| TACSTD2 | CD83 | MSMB |
| TACSTD2 | CD83 | PROK1 |
| TACSTD2 | CD83 | SIT1 |
| TACSTD2 | CD83 | SKAP1 |
| TACSTD2 | CD83 | SPINK5 |
| TACSTD2 | CD83 | TANK |
| TACSTD2 | CD83 | WFDC2 |
| TACSTD2 | CD99L2 | CLEC4D |
| TACSTD2 | CD99L2 | CLEC6A |
| TACSTD2 | CD99L2 | CLMP |
| TACSTD2 | CD99L2 | CPE |
| TACSTD2 | CD99L2 | FR-alpha |
| TACSTD2 | CD99L2 | ICOSLG |
| TACSTD2 | CD99L2 | IRF9 |
| TACSTD2 | CD99L2 | LAMP3 |
| TACSTD2 | CD99L2 | MSMB |
| TACSTD2 | CD99L2 | PROK1 |
| TACSTD2 | CD99L2 | SIT1 |
| TACSTD2 | CD99L2 | SKAP1 |
| TACSTD2 | CD99L2 | SPINK5 |
| TACSTD2 | CD99L2 | TANK |
| TACSTD2 | CD99L2 | WFDC2 |
| TACSTD2 | CLEC4D | CLEC6A |
| TACSTD2 | CLEC4D | CLMP |
| TACSTD2 | CLEC4D | CPE |
| TACSTD2 | CLEC4D | FR-alpha |
| TACSTD2 | CLEC4D | ICOSLG |
| TACSTD2 | CLEC4D | IRF9 |
| TACSTD2 | CLEC4D | LAMP3 |
| TACSTD2 | CLEC4D | MSMB |
| TACSTD2 | CLEC4D | PROK1 |
| TACSTD2 | CLEC4D | SIT1 |
| TACSTD2 | CLEC4D | SKAP1 |
| TACSTD2 | CLEC4D | SPINK5 |
| TACSTD2 | CLEC4D | TANK |
| TACSTD2 | CLEC4D | WFDC2 |
| TACSTD2 | CLEC6A | CLMP |
| TACSTD2 | CLEC6A | CPE |
| TACSTD2 | CLEC6A | FR-alpha |
| TACSTD2 | CLEC6A | ICOSLG |
| TACSTD2 | CLEC6A | IRF9 |
| TACSTD2 | CLEC6A | LAMP3 |
| TACSTD2 | CLEC6A | MSMB |
| TACSTD2 | CLEC6A | PROK1 |
| TACSTD2 | CLEC6A | SIT1 |
| TACSTD2 | CLEC6A | SKAP1 |
| TACSTD2 | CLEC6A | SPINK5 |
| TACSTD2 | CLEC6A | TANK |
| TACSTD2 | CLEC6A | WFDC2 |
| TACSTD2 | CLMP | CPE |
| TACSTD2 | CLMP | FR-alpha |
| TACSTD2 | CLMP | ICOSLG |
| TACSTD2 | CLMP | IRF9 |
| TACSTD2 | CLMP | LAMP3 |
| TACSTD2 | CLMP | MSMB |
| TACSTD2 | CLMP | PROK1 |
| TACSTD2 | CLMP | SIT1 |
| TACSTD2 | CLMP | SKAP1 |
| TACSTD2 | CLMP | SPINK5 |
| TACSTD2 | CLMP | TANK |
| TACSTD2 | CLMP | WFDC2 |
| TACSTD2 | CPE | FR-alpha |
| TACSTD2 | CPE | ICOSLG |
| TACSTD2 | CPE | IRF9 |
| TACSTD2 | CPE | LAMP3 |
| TACSTD2 | CPE | MSMB |
| TACSTD2 | CPE | PROK1 |
| TACSTD2 | CPE | SIT1 |
| TACSTD2 | CPE | SKAP1 |
| TACSTD2 | CPE | SPINK5 |
| TACSTD2 | CPE | TANK |
| TACSTD2 | CPE | WFDC2 |
| TACSTD2 | FR-alpha | ICOSLG |
| TACSTD2 | FR-alpha | IRF9 |
| TACSTD2 | FR-alpha | LAMP3 |
| TACSTD2 | FR-alpha | MSMB |
| TACSTD2 | FR-alpha | PROK1 |
| TACSTD2 | FR-alpha | SIT1 |
| TACSTD2 | FR-alpha | SKAP1 |
| TACSTD2 | FR-alpha | SPINK5 |
| TACSTD2 | FR-alpha | TANK |
| TACSTD2 | FR-alpha | WFDC2 |
| TACSTD2 | ICOSLG | IRF9 |
| TACSTD2 | ICOSLG | LAMP3 |
| TACSTD2 | ICOSLG | MSMB |
| TACSTD2 | ICOSLG | PROK1 |
| TACSTD2 | ICOSLG | SIT1 |
| TACSTD2 | ICOSLG | SKAP1 |
| TACSTD2 | ICOSLG | SPINK5 |
| TACSTD2 | ICOSLG | TANK |
| TACSTD2 | ICOSLG | WFDC2 |
| TACSTD2 | IRF9 | LAMP3 |
| TACSTD2 | IRF9 | MSMB |
| TACSTD2 | IRF9 | PROK1 |
| TACSTD2 | IRF9 | SIT1 |
| TACSTD2 | IRF9 | SKAP1 |
| TACSTD2 | IRF9 | SPINK5 |
| TACSTD2 | IRF9 | TANK |
| TACSTD2 | IRF9 | WFDC2 |
| TACSTD2 | LAMP3 | MSMB |
| TACSTD2 | LAMP3 | PROK1 |
| TACSTD2 | LAMP3 | SIT1 |
| TACSTD2 | LAMP3 | SKAP1 |
| TACSTD2 | LAMP3 | SPINK5 |
| TACSTD2 | LAMP3 | TANK |
| TACSTD2 | LAMP3 | WFDC2 |
| TACSTD2 | MSMB | PROK1 |
| TACSTD2 | MSMB | SIT1 |
| TACSTD2 | MSMB | SKAP1 |
| TACSTD2 | MSMB | SPINK5 |
| TACSTD2 | MSMB | TANK |
| TACSTD2 | MSMB | WFDC2 |
| TACSTD2 | PROK1 | SIT1 |
| TACSTD2 | PROK1 | SKAP1 |
| TACSTD2 | PROK1 | SPINK5 |
| TACSTD2 | PROK1 | TANK |
| TACSTD2 | PROK1 | WFDC2 |
| TACSTD2 | SIT1 | SKAP1 |
| TACSTD2 | SIT1 | SPINK5 |
| TACSTD2 | SIT1 | TANK |
| TACSTD2 | SIT1 | WFDC2 |
| TACSTD2 | SKAP1 | SPINK5 |
| TACSTD2 | SKAP1 | TANK |
| TACSTD2 | SKAP1 | WFDC2 |
| TACSTD2 | SPINK5 | TANK |
| TACSTD2 | SPINK5 | WFDC2 |
| TACSTD2 | TANK | WFDC2 |
| CD83 | CD99L2 | CLEC4D |
| CD83 | CD99L2 | CLEC6A |
| CD83 | CD99L2 | CLMP |
| CD83 | CD99L2 | CPE |
| CD83 | CD99L2 | FR-alpha |
| CD83 | CD99L2 | ICOSLG |
| CD83 | CD99L2 | IRF9 |
| CD83 | CD99L2 | LAMP3 |
| CD83 | CD99L2 | MSMB |
| CD83 | CD99L2 | PROK1 |
| CD83 | CD99L2 | SIT1 |
| CD83 | CD99L2 | SKAP1 |
| CD83 | CD99L2 | SPINK5 |
| CD83 | CD99L2 | TANK |
| CD83 | CD99L2 | WFDC2 |
| CD83 | CLEC4D | CLEC6A |
| CD83 | CLEC4D | CLMP |
| CD83 | CLEC4D | CPE |
| CD83 | CLEC4D | FR-alpha |
| CD83 | CLEC4D | ICOSLG |
| CD83 | CLEC4D | IRF9 |
| CD83 | CLEC4D | LAMP3 |
| CD83 | CLEC4D | MSMB |
| CD83 | CLEC4D | PROK1 |
| CD83 | CLEC4D | SIT1 |
| CD83 | CLEC4D | SKAP1 |
| CD83 | CLEC4D | SPINK5 |
| CD83 | CLEC4D | TANK |
| CD83 | CLEC4D | WFDC2 |
| CD83 | CLEC6A | CLMP |
| CD83 | CLEC6A | CPE |
| CD83 | CLEC6A | FR-alpha |
| CD83 | CLEC6A | ICOSLG |
| CD83 | CLEC6A | IRF9 |
| CD83 | CLEC6A | LAMP3 |
| CD83 | CLEC6A | MSMB |
| CD83 | CLEC6A | PROK1 |
| CD83 | CLEC6A | SIT1 |
| CD83 | CLEC6A | SKAP1 |
| CD83 | CLEC6A | SPINK5 |
| CD83 | CLEC6A | TANK |
| CD83 | CLEC6A | WFDC2 |
| CD83 | CLMP | CPE |
| CD83 | CLMP | FR-alpha |
| CD83 | CLMP | ICOSLG |
| CD83 | CLMP | IRF9 |
| CD83 | CLMP | LAMP3 |
| CD83 | CLMP | MSMB |
| CD83 | CLMP | PROK1 |
| CD83 | CLMP | SIT1 |
| CD83 | CLMP | SKAP1 |
| CD83 | CLMP | SPINK5 |
| CD83 | CLMP | TANK |
| CD83 | CLMP | WFDC2 |
| CD83 | CPE | FR-alpha |
| CD83 | CPE | ICOSLG |
| CD83 | CPE | IRF9 |
| CD83 | CPE | LAMP3 |
| CD83 | CPE | MSMB |
| CD83 | CPE | PROK1 |
| CD83 | CPE | SIT1 |
| CD83 | CPE | SKAP1 |
| CD83 | CPE | SPINK5 |
| CD83 | CPE | TANK |
| CD83 | CPE | WFDC2 |
| CD83 | FR-alpha | ICOSLG |
| CD83 | FR-alpha | IRF9 |
| CD83 | FR-alpha | LAMP3 |
| CD83 | FR-alpha | MSMB |
| CD83 | FR-alpha | PROK1 |
| CD83 | FR-alpha | SIT1 |
| CD83 | FR-alpha | SKAP1 |
| CD83 | FR-alpha | SPINK5 |
| CD83 | FR-alpha | TANK |
| CD83 | FR-alpha | WFDC2 |
| CD83 | ICOSLG | IRF9 |
| CD83 | ICOSLG | LAMP3 |
| CD83 | ICOSLG | MSMB |
| CD83 | ICOSLG | PROK1 |
| CD83 | ICOSLG | SIT1 |
| CD83 | ICOSLG | SKAP1 |
| CD83 | ICOSLG | SPINK5 |
| CD83 | ICOSLG | TANK |
| CD83 | ICOSLG | WFDC2 |
| CD83 | IRF9 | LAMP3 |
| CD83 | IRF9 | MSMB |
| CD83 | IRF9 | PROK1 |
| CD83 | IRF9 | SIT1 |
| CD83 | IRF9 | SKAP1 |
| CD83 | IRF9 | SPINK5 |
| CD83 | IRF9 | TANK |
| CD83 | IRF9 | WFDC2 |
| CD83 | LAMP3 | MSMB |
| CD83 | LAMP3 | PROK1 |
| CD83 | LAMP3 | SIT1 |
| CD83 | LAMP3 | SKAP1 |
| CD83 | LAMP3 | SPINK5 |
| CD83 | LAMP3 | TANK |
| CD83 | LAMP3 | WFDC2 |
| CD83 | MSMB | PROK1 |
| CD83 | MSMB | SIT1 |
| CD83 | MSMB | SKAP1 |
| CD83 | MSMB | SPINK5 |
| CD83 | MSMB | TANK |
| CD83 | MSMB | WFDC2 |
| CD83 | PROK1 | SIT1 |
| CD83 | PROK1 | SKAP1 |
| CD83 | PROK1 | SPINK5 |
| CD83 | PROK1 | TANK |
| CD83 | PROK1 | WFDC2 |
| CD83 | SIT1 | SKAP1 |
| CD83 | SIT1 | SPINK5 |
| CD83 | SIT1 | TANK |
| CD83 | SIT1 | WFDC2 |
| CD83 | SKAP1 | SPINK5 |
| CD83 | SKAP1 | TANK |
| CD83 | SKAP1 | WFDC2 |
| CD83 | SPINK5 | TANK |
| CD83 | SPINK5 | WFDC2 |
| CD83 | TANK | WFDC2 |
| CD99L2 | CLEC4D | CLEC6A |
| CD99L2 | CLEC4D | CLMP |
| CD99L2 | CLEC4D | CPE |
| CD99L2 | CLEC4D | FR-alpha |
| CD99L2 | CLEC4D | ICOSLG |
| CD99L2 | CLEC4D | IRF9 |
| CD99L2 | CLEC4D | LAMP3 |
| CD99L2 | CLEC4D | MSMB |
| CD99L2 | CLEC4D | PROK1 |
| CD99L2 | CLEC4D | SIT1 |
| CD99L2 | CLEC4D | SKAP1 |
| CD99L2 | CLEC4D | SPINK5 |
| CD99L2 | CLEC4D | TANK |
| CD99L2 | CLEC4D | WFDC2 |
| CD99L2 | CLEC6A | CLMP |
| CD99L2 | CLEC6A | CPE |
| CD99L2 | CLEC6A | FR-alpha |
| CD99L2 | CLEC6A | ICOSLG |
| CD99L2 | CLEC6A | IRF9 |
| CD99L2 | CLEC6A | LAMP3 |
| CD99L2 | CLEC6A | MSMB |
| CD99L2 | CLEC6A | PROK1 |
| CD99L2 | CLEC6A | SIT1 |
| CD99L2 | CLEC6A | SKAP1 |
| CD99L2 | CLEC6A | SPINK5 |
| CD99L2 | CLEC6A | TANK |
| CD99L2 | CLEC6A | WFDC2 |
| CD99L2 | CLMP | CPE |
| CD99L2 | CLMP | FR-alpha |
| CD99L2 | CLMP | ICOSLG |
| CD99L2 | CLMP | IRF9 |
| CD99L2 | CLMP | LAMP3 |
| CD99L2 | CLMP | MSMB |
| CD99L2 | CLMP | PROK1 |
| CD99L2 | CLMP | SIT1 |
| CD99L2 | CLMP | SKAP1 |
| CD99L2 | CLMP | SPINK5 |
| CD99L2 | CLMP | TANK |
| CD99L2 | CLMP | WFDC2 |
| CD99L2 | CPE | FR-alpha |
| CD99L2 | CPE | ICOSLG |
| CD99L2 | CPE | IRF9 |
| CD99L2 | CPE | LAMP3 |
| CD99L2 | CPE | MSMB |
| CD99L2 | CPE | PROK1 |
| CD99L2 | CPE | SIT1 |
| CD99L2 | CPE | SKAP1 |
| CD99L2 | CPE | SPINK5 |
| CD99L2 | CPE | TANK |
| CD99L2 | CPE | WFDC2 |
| CD99L2 | FR-alpha | ICOSLG |
| CD99L2 | FR-alpha | IRF9 |
| CD99L2 | FR-alpha | LAMP3 |
| CD99L2 | FR-alpha | MSMB |
| CD99L2 | FR-alpha | PROK1 |
| CD99L2 | FR-alpha | SIT1 |
| CD99L2 | FR-alpha | SKAP1 |
| CD99L2 | FR-alpha | SPINK5 |
| CD99L2 | FR-alpha | TANK |
| CD99L2 | FR-alpha | WFDC2 |
| CD99L2 | ICOSLG | IRF9 |
| CD99L2 | ICOSLG | LAMP3 |
| CD99L2 | ICOSLG | MSMB |
| CD99L2 | ICOSLG | PROK1 |
| CD99L2 | ICOSLG | SIT1 |
| CD99L2 | ICOSLG | SKAP1 |
| CD99L2 | ICOSLG | SPINK5 |
| CD99L2 | ICOSLG | TANK |
| CD99L2 | ICOSLG | WFDC2 |
| CD99L2 | IRF9 | LAMP3 |
| CD99L2 | IRF9 | MSMB |
| CD99L2 | IRF9 | PROK1 |
| CD99L2 | IRF9 | SIT1 |
| CD99L2 | IRF9 | SKAP1 |
| CD99L2 | IRF9 | SPINK5 |
| CD99L2 | IRF9 | TANK |
| CD99L2 | IRF9 | WFDC2 |
| CD99L2 | LAMP3 | MSMB |
| CD99L2 | LAMP3 | PROK1 |
| CD99L2 | LAMP3 | SIT1 |
| CD99L2 | LAMP3 | SKAP1 |
| CD99L2 | LAMP3 | SPINK5 |
| CD99L2 | LAMP3 | TANK |
| CD99L2 | LAMP3 | WFDC2 |
| CD99L2 | MSMB | PROK1 |
| CD99L2 | MSMB | SIT1 |
| CD99L2 | MSMB | SKAP1 |
| CD99L2 | MSMB | SPINK5 |
| CD99L2 | MSMB | TANK |
| CD99L2 | MSMB | WFDC2 |
| CD99L2 | PROK1 | SIT1 |
| CD99L2 | PROK1 | SKAP1 |
| CD99L2 | PROK1 | SPINK5 |
| CD99L2 | PROK1 | TANK |
| CD99L2 | PROK1 | WFDC2 |
| CD99L2 | SIT1 | SKAP1 |
| CD99L2 | SIT1 | SPINK5 |
| CD99L2 | SIT1 | TANK |
| CD99L2 | SIT1 | WFDC2 |
| CD99L2 | SKAP1 | SPINK5 |
| CD99L2 | SKAP1 | TANK |
| CD99L2 | SKAP1 | WFDC2 |
| CD99L2 | SPINK5 | TANK |
| CD99L2 | SPINK5 | WFDC2 |
| CD99L2 | TANK | WFDC2 |
| CLEC4D | CLEC6A | CLMP |
| CLEC4D | CLEC6A | CPE |
| CLEC4D | CLEC6A | FR-alpha |
| CLEC4D | CLEC6A | ICOSLG |
| CLEC4D | CLEC6A | IRF9 |
| CLEC4D | CLEC6A | LAMP3 |
| CLEC4D | CLEC6A | MSMB |
| CLEC4D | CLEC6A | PROK1 |
| CLEC4D | CLEC6A | SIT1 |
| CLEC4D | CLEC6A | SKAP1 |
| CLEC4D | CLEC6A | SPINK5 |
| CLEC4D | CLEC6A | TANK |
| CLEC4D | CLEC6A | WFDC2 |
| CLEC4D | CLMP | CPE |
| CLEC4D | CLMP | FR-alpha |
| CLEC4D | CLMP | ICOSLG |
| CLEC4D | CLMP | IRF9 |
| CLEC4D | CLMP | LAMP3 |
| CLEC4D | CLMP | MSMB |
| CLEC4D | CLMP | PROK1 |
| CLEC4D | CLMP | SIT1 |
| CLEC4D | CLMP | SKAP1 |
| CLEC4D | CLMP | SPINK5 |
| CLEC4D | CLMP | TANK |
| CLEC4D | CLMP | WFDC2 |
| CLEC4D | CPE | FR-alpha |
| CLEC4D | CPE | ICOSLG |
| CLEC4D | CPE | IRF9 |
| CLEC4D | CPE | LAMP3 |
| CLEC4D | CPE | MSMB |
| CLEC4D | CPE | PROK1 |
| CLEC4D | CPE | SIT1 |
| CLEC4D | CPE | SKAP1 |
| CLEC4D | CPE | SPINK5 |
| CLEC4D | CPE | TANK |
| CLEC4D | CPE | WFDC2 |
| CLEC4D | FR-alpha | ICOSLG |
| CLEC4D | FR-alpha | IRF9 |
| CLEC4D | FR-alpha | LAMP3 |
| CLEC4D | FR-alpha | MSMB |
| CLEC4D | FR-alpha | PROK1 |
| CLEC4D | FR-alpha | SIT1 |
| CLEC4D | FR-alpha | SKAP1 |
| CLEC4D | FR-alpha | SPINK5 |
| CLEC4D | FR-alpha | TANK |
| CLEC4D | FR-alpha | WFDC2 |
| CLEC4D | ICOSLG | IRF9 |
| CLEC4D | ICOSLG | LAMP3 |
| CLEC4D | ICOSLG | MSMB |
| CLEC4D | ICOSLG | PROK1 |
| CLEC4D | ICOSLG | SIT1 |
| CLEC4D | ICOSLG | SKAP1 |
| CLEC4D | ICOSLG | SPINK5 |
| CLEC4D | ICOSLG | TANK |
| CLEC4D | ICOSLG | WFDC2 |
| CLEC4D | IRF9 | LAMP3 |
| CLEC4D | IRF9 | MSMB |
| CLEC4D | IRF9 | PROK1 |
| CLEC4D | IRF9 | SIT1 |
| CLEC4D | IRF9 | SKAP1 |
| CLEC4D | IRF9 | SPINK5 |
| CLEC4D | IRF9 | TANK |
| CLEC4D | IRF9 | WFDC2 |
| CLEC4D | LAMP3 | MSMB |
| CLEC4D | LAMP3 | PROK1 |
| CLEC4D | LAMP3 | SIT1 |
| CLEC4D | LAMP3 | SKAP1 |
| CLEC4D | LAMP3 | SPINK5 |
| CLEC4D | LAMP3 | TANK |
| CLEC4D | LAMP3 | WFDC2 |
| CLEC4D | MSMB | PROK1 |
| CLEC4D | MSMB | SIT1 |
| CLEC4D | MSMB | SKAP1 |
| CLEC4D | MSMB | SPINK5 |
| CLEC4D | MSMB | TANK |
| CLEC4D | MSMB | WFDC2 |
| CLEC4D | PROK1 | SIT1 |
| CLEC4D | PROK1 | SKAP1 |
| CLEC4D | PROK1 | SPINK5 |
| CLEC4D | PROK1 | TANK |
| CLEC4D | PROK1 | WFDC2 |
| CLEC4D | SIT1 | SKAP1 |
| CLEC4D | SIT1 | SPINK5 |
| CLEC4D | SIT1 | TANK |
| CLEC4D | SIT1 | WFDC2 |
| CLEC4D | SKAP1 | SPINK5 |
| CLEC4D | SKAP1 | TANK |
| CLEC4D | SKAP1 | WFDC2 |
| CLEC4D | SPINK5 | TANK |
| CLEC4D | SPINK5 | WFDC2 |
| CLEC4D | TANK | WFDC2 |
| CLEC6A | CLMP | CPE |
| CLEC6A | CLMP | FR-alpha |
| CLEC6A | CLMP | ICOSLG |
| CLEC6A | CLMP | IRF9 |
| CLEC6A | CLMP | LAMP3 |
| CLEC6A | CLMP | MSMB |
| CLEC6A | CLMP | PROK1 |
| CLEC6A | CLMP | SIT1 |
| CLEC6A | CLMP | SKAP1 |
| CLEC6A | CLMP | SPINK5 |
| CLEC6A | CLMP | TANK |
| CLEC6A | CLMP | WFDC2 |
| CLEC6A | CPE | FR-alpha |
| CLEC6A | CPE | ICOSLG |
| CLEC6A | CPE | IRF9 |
| CLEC6A | CPE | LAMP3 |
| CLEC6A | CPE | MSMB |
| CLEC6A | CPE | PROK1 |
| CLEC6A | CPE | SIT1 |
| CLEC6A | CPE | SKAP1 |
| CLEC6A | CPE | SPINK5 |
| CLEC6A | CPE | TANK |
| CLEC6A | CPE | WFDC2 |
| CLEC6A | FR-alpha | ICOSLG |
| CLEC6A | FR-alpha | IRF9 |
| CLEC6A | FR-alpha | LAMP3 |
| CLEC6A | FR-alpha | MSMB |
| CLEC6A | FR-alpha | PROK1 |
| CLEC6A | FR-alpha | SIT1 |
| CLEC6A | FR-alpha | SKAP1 |
| CLEC6A | FR-alpha | SPINK5 |
| CLEC6A | FR-alpha | TANK |
| CLEC6A | FR-alpha | WFDC2 |
| CLEC6A | ICOSLG | IRF9 |
| CLEC6A | ICOSLG | LAMP3 |
| CLEC6A | ICOSLG | MSMB |
| CLEC6A | ICOSLG | PROK1 |
| CLEC6A | ICOSLG | SIT1 |
| CLEC6A | ICOSLG | SKAP1 |
| CLEC6A | ICOSLG | SPINK5 |
| CLEC6A | ICOSLG | TANK |
| CLEC6A | ICOSLG | WFDC2 |
| CLEC6A | IRF9 | LAMP3 |
| CLEC6A | IRF9 | MSMB |
| CLEC6A | IRF9 | PROK1 |
| CLEC6A | IRF9 | SIT1 |
| CLEC6A | IRF9 | SKAP1 |
| CLEC6A | IRF9 | SPINK5 |
| CLEC6A | IRF9 | TANK |
| CLEC6A | IRF9 | WFDC2 |
| CLEC6A | LAMP3 | MSMB |
| CLEC6A | LAMP3 | PROK1 |
| CLEC6A | LAMP3 | SIT1 |
| CLEC6A | LAMP3 | SKAP1 |
| CLEC6A | LAMP3 | SPINK5 |
| CLEC6A | LAMP3 | TANK |
| CLEC6A | LAMP3 | WFDC2 |
| CLEC6A | MSMB | PROK1 |
| CLEC6A | MSMB | SIT1 |
| CLEC6A | MSMB | SKAP1 |
| CLEC6A | MSMB | SPINK5 |
| CLEC6A | MSMB | TANK |
| CLEC6A | MSMB | WFDC2 |
| CLEC6A | PROK1 | SIT1 |
| CLEC6A | PROK1 | SKAP1 |
| CLEC6A | PROK1 | SPINK5 |
| CLEC6A | PROK1 | TANK |
| CLEC6A | PROK1 | WFDC2 |
| CLEC6A | SIT1 | SKAP1 |
| CLEC6A | SIT1 | SPINK5 |
| CLEC6A | SIT1 | TANK |
| CLEC6A | SIT1 | WFDC2 |
| CLEC6A | SKAP1 | SPINK5 |
| CLEC6A | SKAP1 | TANK |
| CLEC6A | SKAP1 | WFDC2 |
| CLEC6A | SPINK5 | TANK |
| CLEC6A | SPINK5 | WFDC2 |
| CLEC6A | TANK | WFDC2 |
| CLMP | CPE | FR-alpha |
| CLMP | CPE | ICOSLG |
| CLMP | CPE | IRF9 |
| CLMP | CPE | LAMP3 |
| CLMP | CPE | MSMB |
| CLMP | CPE | PROK1 |
| CLMP | CPE | SIT1 |
| CLMP | CPE | SKAP1 |
| CLMP | CPE | SPINK5 |
| CLMP | CPE | TANK |
| CLMP | CPE | WFDC2 |
| CLMP | FR-alpha | ICOSLG |
| CLMP | FR-alpha | IRF9 |
| CLMP | FR-alpha | LAMP3 |
| CLMP | FR-alpha | MSMB |
| CLMP | FR-alpha | PROK1 |
| CLMP | FR-alpha | SIT1 |
| CLMP | FR-alpha | SKAP1 |
| CLMP | FR-alpha | SPINK5 |
| CLMP | FR-alpha | TANK |
| CLMP | FR-alpha | WFDC2 |
| CLMP | ICOSLG | IRF9 |
| CLMP | ICOSLG | LAMP3 |
| CLMP | ICOSLG | MSMB |
| CLMP | ICOSLG | PROK1 |
| CLMP | ICOSLG | SIT1 |
| CLMP | ICOSLG | SKAP1 |
| CLMP | ICOSLG | SPINK5 |
| CLMP | ICOSLG | TANK |
| CLMP | ICOSLG | WFDC2 |
| CLMP | IRF9 | LAMP3 |
| CLMP | IRF9 | MSMB |
| CLMP | IRF9 | PROK1 |
| CLMP | IRF9 | SIT1 |
| CLMP | IRF9 | SKAP1 |
| CLMP | IRF9 | SPINK5 |
| CLMP | IRF9 | TANK |
| CLMP | IRF9 | WFDC2 |
| CLMP | LAMP3 | MSMB |
| CLMP | LAMP3 | PROK1 |
| CLMP | LAMP3 | SIT1 |
| CLMP | LAMP3 | SKAP1 |
| CLMP | LAMP3 | SPINK5 |
| CLMP | LAMP3 | TANK |
| CLMP | LAMP3 | WFDC2 |
| CLMP | MSMB | PROK1 |
| CLMP | MSMB | SIT1 |
| CLMP | MSMB | SKAP1 |
| CLMP | MSMB | SPINK5 |
| CLMP | MSMB | TANK |
| CLMP | MSMB | WFDC2 |
| CLMP | PROK1 | SIT1 |
| CLMP | PROK1 | SKAP1 |
| CLMP | PROK1 | SPINK5 |
| CLMP | PROK1 | TANK |
| CLMP | PROK1 | WFDC2 |
| CLMP | SIT1 | SKAP1 |
| CLMP | SIT1 | SPINK5 |
| CLMP | SIT1 | TANK |
| CLMP | SIT1 | WFDC2 |
| CLMP | SKAP1 | SPINK5 |
| CLMP | SKAP1 | TANK |
| CLMP | SKAP1 | WFDC2 |
| CLMP | SPINK5 | TANK |
| CLMP | SPINK5 | WFDC2 |
| CLMP | TANK | WFDC2 |
| CPE | FR-alpha | ICOSLG |
| CPE | FR-alpha | IRF9 |
| CPE | FR-alpha | LAMP3 |
| CPE | FR-alpha | MSMB |
| CPE | FR-alpha | PROK1 |
| CPE | FR-alpha | SIT1 |
| CPE | FR-alpha | SKAP1 |
| CPE | FR-alpha | SPINK5 |
| CPE | FR-alpha | TANK |
| CPE | FR-alpha | WFDC2 |
| CPE | ICOSLG | IRF9 |
| CPE | ICOSLG | LAMP3 |
| CPE | ICOSLG | MSMB |
| CPE | ICOSLG | PROK1 |
| CPE | ICOSLG | SIT1 |
| CPE | ICOSLG | SKAP1 |
| CPE | ICOSLG | SPINK5 |
| CPE | ICOSLG | TANK |
| CPE | ICOSLG | WFDC2 |
| CPE | IRF9 | LAMP3 |
| CPE | IRF9 | MSMB |
| CPE | IRF9 | PROK1 |
| CPE | IRF9 | SIT1 |
| CPE | IRF9 | SKAP1 |
| CPE | IRF9 | SPINK5 |
| CPE | IRF9 | TANK |
| CPE | IRF9 | WFDC2 |
| CPE | LAMP3 | MSMB |
| CPE | LAMP3 | PROK1 |
| CPE | LAMP3 | SIT1 |
| CPE | LAMP3 | SKAP1 |
| CPE | LAMP3 | SPINK5 |
| CPE | LAMP3 | TANK |
| CPE | LAMP3 | WFDC2 |
| CPE | MSMB | PROK1 |
| CPE | MSMB | SIT1 |
| CPE | MSMB | SKAP1 |
| CPE | MSMB | SPINK5 |
| CPE | MSMB | TANK |
| CPE | MSMB | WFDC2 |
| CPE | PROK1 | SIT1 |
| CPE | PROK1 | SKAP1 |
| CPE | PROK1 | SPINK5 |
| CPE | PROK1 | TANK |
| CPE | PROK1 | WFDC2 |
| CPE | SIT1 | SKAP1 |
| CPE | SIT1 | SPINK5 |
| CPE | SIT1 | TANK |
| CPE | SIT1 | WFDC2 |
| CPE | SKAP1 | SPINK5 |
| CPE | SKAP1 | TANK |
| CPE | SKAP1 | WFDC2 |
| CPE | SPINK5 | TANK |
| CPE | SPINK5 | WFDC2 |
| CPE | TANK | WFDC2 |
| FR-alpha | ICOSLG | IRF9 |
| FR-alpha | ICOSLG | LAMP3 |
| FR-alpha | ICOSLG | MSMB |
| FR-alpha | ICOSLG | PROK1 |
| FR-alpha | ICOSLG | SIT1 |
| FR-alpha | ICOSLG | SKAP1 |
| FR-alpha | ICOSLG | SPINK5 |
| FR-alpha | ICOSLG | TANK |
| FR-alpha | ICOSLG | WFDC2 |
| FR-alpha | IRF9 | LAMP3 |
| FR-alpha | IRF9 | MSMB |
| FR-alpha | IRF9 | PROK1 |
| FR-alpha | IRF9 | SIT1 |
| FR-alpha | IRF9 | SKAP1 |
| FR-alpha | IRF9 | SPINK5 |
| FR-alpha | IRF9 | TANK |
| FR-alpha | IRF9 | WFDC2 |
| FR-alpha | LAMP3 | MSMB |
| FR-alpha | LAMP3 | PROK1 |
| FR-alpha | LAMP3 | SIT1 |
| FR-alpha | LAMP3 | SKAP1 |
| FR-alpha | LAMP3 | SPINK5 |
| FR-alpha | LAMP3 | TANK |
| FR-alpha | LAMP3 | WFDC2 |
| FR-alpha | MSMB | PROK1 |
| FR-alpha | MSMB | SIT1 |
| FR-alpha | MSMB | SKAP1 |
| FR-alpha | MSMB | SPINK5 |
| FR-alpha | MSMB | TANK |
| FR-alpha | MSMB | WFDC2 |
| FR-alpha | PROK1 | SIT1 |
| FR-alpha | PROK1 | SKAP1 |
| FR-alpha | PROK1 | SPINK5 |
| FR-alpha | PROK1 | TANK |
| FR-alpha | PROK1 | WFDC2 |
| FR-alpha | SIT1 | SKAP1 |
| FR-alpha | SIT1 | SPINK5 |
| FR-alpha | SIT1 | TANK |
| FR-alpha | SIT1 | WFDC2 |
| FR-alpha | SKAP1 | SPINK5 |
| FR-alpha | SKAP1 | TANK |
| FR-alpha | SKAP1 | WFDC2 |
| FR-alpha | SPINK5 | TANK |
| FR-alpha | SPINK5 | WFDC2 |
| FR-alpha | TANK | WFDC2 |
| ICOSLG | IRF9 | LAMP3 |
| ICOSLG | IRF9 | MSMB |
| ICOSLG | IRF9 | PROK1 |
| ICOSLG | IRF9 | SIT1 |
| ICOSLG | IRF9 | SKAP1 |
| ICOSLG | IRF9 | SPINK5 |
| ICOSLG | IRF9 | TANK |
| ICOSLG | IRF9 | WFDC2 |
| ICOSLG | LAMP3 | MSMB |
| ICOSLG | LAMP3 | PROK1 |
| ICOSLG | LAMP3 | SIT1 |
| ICOSLG | LAMP3 | SKAP1 |
| ICOSLG | LAMP3 | SPINK5 |
| ICOSLG | LAMP3 | TANK |
| ICOSLG | LAMP3 | WFDC2 |
| ICOSLG | MSMB | PROK1 |
| ICOSLG | MSMB | SIT1 |
| ICOSLG | MSMB | SKAP1 |
| ICOSLG | MSMB | SPINK5 |
| ICOSLG | MSMB | TANK |
| ICOSLG | MSMB | WFDC2 |
| ICOSLG | PROK1 | SIT1 |
| ICOSLG | PROK1 | SKAP1 |
| ICOSLG | PROK1 | SPINK5 |
| ICOSLG | PROK1 | TANK |
| ICOSLG | PROK1 | WFDC2 |
| ICOSLG | SIT1 | SKAP1 |
| ICOSLG | SIT1 | SPINK5 |
| ICOSLG | SIT1 | TANK |
| ICOSLG | SIT1 | WFDC2 |
| ICOSLG | SKAP1 | SPINK5 |
| ICOSLG | SKAP1 | TANK |
| ICOSLG | SKAP1 | WFDC2 |
| ICOSLG | SPINK5 | TANK |
| ICOSLG | SPINK5 | WFDC2 |
| ICOSLG | TANK | WFDC2 |
| IRF9 | LAMP3 | MSMB |
| IRF9 | LAMP3 | PROK1 |
| IRF9 | LAMP3 | SIT1 |
| IRF9 | LAMP3 | SKAP1 |
| IRF9 | LAMP3 | SPINK5 |
| IRF9 | LAMP3 | TANK |
| IRF9 | LAMP3 | WFDC2 |
| IRF9 | MSMB | PROK1 |
| IRF9 | MSMB | SIT1 |
| IRF9 | MSMB | SKAP1 |
| IRF9 | MSMB | SPINK5 |
| IRF9 | MSMB | TANK |
| IRF9 | MSMB | WFDC2 |
| IRF9 | PROK1 | SIT1 |
| IRF9 | PROK1 | SKAP1 |
| IRF9 | PROK1 | SPINK5 |
| IRF9 | PROK1 | TANK |
| IRF9 | PROK1 | WFDC2 |
| IRF9 | SIT1 | SKAP1 |
| IRF9 | SIT1 | SPINK5 |
| IRF9 | SIT1 | TANK |
| IRF9 | SIT1 | WFDC2 |
| IRF9 | SKAP1 | SPINK5 |
| IRF9 | SKAP1 | TANK |
| IRF9 | SKAP1 | WFDC2 |
| IRF9 | SPINK5 | TANK |
| IRF9 | SPINK5 | WFDC2 |
| IRF9 | TANK | WFDC2 |
| LAMP3 | MSMB | PROK1 |
| LAMP3 | MSMB | SIT1 |
| LAMP3 | MSMB | SKAP1 |
| LAMP3 | MSMB | SPINK5 |
| LAMP3 | MSMB | TANK |
| LAMP3 | MSMB | WFDC2 |
| LAMP3 | PROK1 | SIT1 |
| LAMP3 | PROK1 | SKAP1 |
| LAMP3 | PROK1 | SPINK5 |
| LAMP3 | PROK1 | TANK |
| LAMP3 | PROK1 | WFDC2 |
| LAMP3 | SIT1 | SKAP1 |
| LAMP3 | SIT1 | SPINK5 |
| LAMP3 | SIT1 | TANK |
| LAMP3 | SIT1 | WFDC2 |
| LAMP3 | SKAP1 | SPINK5 |
| LAMP3 | SKAP1 | TANK |
| LAMP3 | SKAP1 | WFDC2 |
| LAMP3 | SPINK5 | TANK |
| LAMP3 | SPINK5 | WFDC2 |
| LAMP3 | TANK | WFDC2 |
| MSMB | PROK1 | SIT1 |
| MSMB | PROK1 | SKAP1 |
| MSMB | PROK1 | SPINK5 |
| MSMB | PROK1 | TANK |
| MSMB | PROK1 | WFDC2 |
| MSMB | SIT1 | SKAP1 |
| MSMB | SIT1 | SPINK5 |
| MSMB | SIT1 | TANK |
| MSMB | SIT1 | WFDC2 |
| MSMB | SKAP1 | SPINK5 |
| MSMB | SKAP1 | TANK |
| MSMB | SKAP1 | WFDC2 |
| MSMB | SPINK5 | TANK |
| MSMB | SPINK5 | WFDC2 |
| MSMB | TANK | WFDC2 |
| PROK1 | SIT1 | SKAP1 |
| PROK1 | SIT1 | SPINK5 |
| PROK1 | SIT1 | TANK |
| PROK1 | SIT1 | WFDC2 |
| PROK1 | SKAP1 | SPINK5 |
| PROK1 | SKAP1 | TANK |
| PROK1 | SKAP1 | WFDC2 |
| PROK1 | SPINK5 | TANK |
| PROK1 | SPINK5 | WFDC2 |
| PROK1 | TANK | WFDC2 |
| SIT1 | SKAP1 | SPINK5 |
| SIT1 | SKAP1 | TANK |
| SIT1 | SKAP1 | WFDC2 |
| SIT1 | SPINK5 | TANK |
| SIT1 | SPINK5 | WFDC2 |
| SIT1 | TANK | WFDC2 |
| SKAP1 | SPINK5 | TANK |
| SKAP1 | SPINK5 | WFDC2 |
| SKAP1 | TANK | WFDC2 |
| SPINK5 | TANK | WFDC2 |

In particular embodiments, the methods of the present invention may comprise determining the level of particular combinations of three biomarkers, wherein said biomarkers are MUC-16 and:
a) TACSTD2, and SPINT1, KRT19 or CLEC4D;
b) TACSTD2, and, ICOSLG, CLEC6A or PROK1;
c) SPINT1, and TACSTD2, ICOSLG or CLEC6A;
d) SPINT1, and ICOSLG, CLEC6A or MSMB;
e) KLK10, and SPINT1, CPE or CLEC6A;
f) PTK7, and SPINT1, CLEC6A or SPINK5;
g) SEZ6L2, and SPINT1, SPINK5 or CLEC6A;
h) KRT19, and SPINT1, ICOSLG or CLEC4D;
i) KRT19, and TACSTD2, SPINT1 or ICOSLG;
j) IL10, and SPINT1, SPINK5 or CLEC6A;
k) SPINT1, and FR-Alpha, TACSTD2 or WFDC2; or
l) TACSTD2, and FR-Alpha, SPINT1 or WFDC2.

Alternatively, the methods of the present invention may comprise determining the level of MUC-16 and:
a) KLK10, and CPE, CLEC6A or TACSTD2;
b) PTK7, and ICOSLG, MSMB or TACSTD2;
c) SEZ6L2, and ICOSLG, CLEC6A or TACSTD2;
d) KRT19, and CLEC4D, ICOSLG or MSMB; or
e) IL10, and ICOSLG, TACSTD2 or CLEC6A.

In more particular embodiments, the methods may comprise determining the level of specific combinations of three biomarkers, wherein said biomarkers are:
a) MUC-16, TACSTD2, and SPINT1;
b) MUC-16, TACSTD2, and, ICOSLG;
c) MUC-16, SPINT1, and ICOSLG;
d) MUC-16, SPINT1 and TACSTD2;
e) MUC-16, KLK10, and SPINT1;
f) MUC-16, PTK7, and SPINT1;
g) MUC-16, SEZ6L2, and SPINT1;
h) MUC-16, KRT19, and SPINT1;
i) MUC-16, KRT19, and TACSTD2;
j) MUC-16, IL10, and SPINT1;
k) MUC-16, SPINT1, and WFDC2;
l) MUC-16, TACSTD2, and WFDC2;
m) MUC-16, SPINT1, and FR-Alpha; or
n) MUC-16, TACSTD2, and FR-Alpha.

Alternatively, the three biomarkers may be:
a) MUC16, KLK10, and CPE;
b) MUC16, PTK7, and ICOSLG;
c) MUC16, SEZ6L2, and ICOSLG;
d) MUC16, KRT19, and CLEC4D; or
e) MUC16, IL10, and ICOSLG.

In an aspect, the present invention may further provide an in vitro method of detecting, predicting or monitoring ovarian cancer in a subject, comprising determining the level of three biomarkers in a sample from said subject, wherein said three biomarkers comprise a first biomarker selected from MUC-16, TACSTD2, and SPINT1 and second and third biomarkers selected from TACSTD2, KRT19, PTK7, SPINT1, MUC-16, KLK10, ICOSLG, IL10, SPINK5, CLEC6A, CLEC4D, CD83, SKAP1 and SEZ6L2, wherein the second and third biomarkers are different from the first biomarker, and from each other, and wherein the biomarkers are not solely MUC-16, IL10 and KLK10.

In a particular embodiment the biomarker levels determined are not solely of any three or all four of the biomarkers MUC-16, IL10, KLK10 and KRT19.

In particular embodiments of this aspect, the three biomarkers may include at least one of TACSTD2, PTK7, SPINT1, ICOSLG, SPINK5, CLEC6A, CLEC4D, CD83, SKAP1 and SEZ6L2, or

at least one of PTK7, SPINT1, ICOSLG, SPINK5, CLEC6A, CLEC4D, CD83, SKAP1 and SEZ6L2, or

at least one of TACSTD2, PTK7, ICOSLG, SPINK5, CLEC6A, CLEC4D, CD83, SKAP1 and SEZ6L2, or

at least one of TACSTD2, PTK7, SPINT1, SPINK5, CLEC6A, CLEC4D, CD83, SKAP1 and SEZ6L2, or

at least one of PTK7, ICOSLG, SPINK5, CLEC6A, CLEC4D, CD83, SKAP1 and SEZ6L2, or

at least one of TACSTD2, PTK7, SPINK5, CLEC6A, CLEC4D, CD83, SKAP1 and SEZ6L2, or

at least one of PTK7, SPINT1, SPINK5, CLEC6A, CLEC4D, CD83, SKAP1 and SEZ6L2, or

at least one of PTK7, SPINK5, CLEC6A, CLEC4D, CD83, SKAP1 and SEZ6L2.

In alternative embodiments, the three biomarkers are:
a) TACSTD2, MUC-16 and SPINT1, ICOSLG, KRT19, KLK10, CLEC6A or SEZ6L2;
b) TACSTD2, ICOSLG and CLEC6A or CLEC4D;
c) TACSTD2, SPINT1 and KRT19, MUC-16 or SPINK5;
d) SPINT1, MUC-16 and TACSTD2, ICOSLG, KRT19 or KLK10;
e) MUC-16, ICOSLG and TACSTD2, CLEC4D, CLEC6A, SPINT1 or SEZ6L2;
f) KRT19, SPINT1 and MUC-16, TACSTD2 or CD83;
g) TACSTD2, KRT19 and MUC-16, SPINT1 or KLK10;
h) TACSTD2, KLK10 and IL10, KRT19, MUC-16 or SKAP1;
i) IL10, MUC-16 and KRT19 or PTK7;
j) SPINT1, SPINK5 and PTK7, IL10 or SEZ6L2; or
k) TACSTD2, IL10 and SKAP1.

More particularly, in representative embodiments, the three biomarkers are:
a) TACSTD2, SPINT1 and MUC-16;
b) SPINT1, MUC-16 and ICOSLG;
c) SPINT1, KRT19 and MUC-16;
d) TACSTD2, SPINT1 and KRT19;
e) TACSTD2, MUC-16 and KRT19;
f) TACSTD2, MUC-16 and ICOSLG;
g) KRT19, IL10 and MUC-16;
h) SPINT1, IL10 and SPINK5;
i) MUC-16, TACSTD2 and CLEC6A;
j) TACSTD2, SPINT1 and SPINK5;
k) TACSTD2, KLK10 and KRT19;
l) SPINT1, KLK10 and MUC-16;
m) MUC-16, ICOSLG and CLEC6A;
n) TACSTD2, ICOSLG and CLEC6A;
o) TACSTD2, KLK10 and IL10;
p) TACSTD2, IL10 and SKAP1;
q) MUC-16, ICOSLG and CLEC4D;
r) TACSTD2, ICOSLG and CLEC4D;
s) SPINT1, KRT10 and CD83;
t) TACSTD2, KLK10 and MUC-16;
u) SPINT1, PTK7 and SPINK5;
v) TACSTD2, KLK10 and SKAP1;
w) SEZ6L2, SPINT1 and SPINK5
x) TACSTD2, SEZ6L2 and MUC-16;
y) SEZ6L2, MUC-16 and ICOSLG; or
z) IL10, PTK7 and MUC-16.

In yet more particular embodiments, the methods may comprise determining the level of combinations of four biomarkers, wherein said biomarkers are:
a) TACSTD2, SPINT1, KRT19 and CLEC4D;
b) TACSTD2, ICOSLG, CLEC6A and PROK1;
c) SPINT1, TACSTD2, ICOSLG and CLEC6A;
d) SPINT1, ICOSLG, CLEC6A and MSMB;
e) KLK10, SPINT1, CPE and CLEC6A;
f) PTK7, SPINT1, CLEC6A and SPINK5;
g) SEZ6L2, SPINT1, SPINK5 and CLEC6A;
h) KRT19, SPINT1, ICOSLG and CLEC4D;
i) KRT19, TACSTD2, SPINT1 and ICOSLG;
j) IL10, SPINT1, SPINK5 and CLEC6A;
k) SPINT1, FR-Alpha, TACSTD2 and WFDC2; or
l) TACSTD2, FR-Alpha, SPINT1 and WFDC2.

Optionally, said methods may further include determining the level of MUC-16 in a sample.

Alternatively, the methods of the present invention may comprise determining the level of:
a) KLK10, CPE, CLEC6A and TACSTD2;
b) PTK7, ICOSLG, MSMB and TACSTD2;
c) SEZ6L2, ICOSLG, CLEC6A and TACSTD2;
d) KRT19, CLEC4D, ICOSLG and MSMB; or
e) IL10, ICOSLG, TACSTD2 and CLEC6A.

Optionally, said methods may further include determining the level of MUC-16 in a sample.

In alternative embodiments, the methods of the present invention may comprise determining the level of combinations of four biomarkers, wherein said biomarkers are:
a) TACSTD2, SPINT1, KRT19 and CLEC4D;
b) TACSTD2, SPINT1, KRT19 and WFDC2;
c) TACSTD2, SPINT1, KRT19 and FR-Alpha;
d) TACSTD2, ICOSLG, CLEC6A and PROK1;
e) SPINT1, ICOSLG, CLEC6A and MSMB;
f) SPINT1, TACSTD2, ICOSLG and CLEC6A;
g) KLK10, SPINT1, CPE and CLEC6A;
h) PTK7, SPINT1, CLEC6A and SPINK5;
i) SEZ6L2, SPINT1, SPINK5 and CLEC6A;
j) KRT19, SPINT1, ICOSLG and CLEC4D;
k) KRT19, TACSTD2, SPINT1 and ICOSLG; or
l) IL10, SPINT1, SPINK5 and CLEC6A.

Optionally, said methods may further include determining the level of MUC-16 in a sample.

Alternatively, the methods may comprise determining the level of:
a) KLK10, CPE, CLEC6A and TACSTD2;
b) PTK7, ICOSLG, MSMB and TACSTD2;
c) SEZ6L2, ICOSLG, MSMB and TACSTD2;
d) KRT19, CLEC4D, ICOSLG and MSMB; or
e) IL10, ICOSLG, TACSTD2 and CLEC6A.

Optionally, said methods may further include determining the level of MUC-16 in a sample.

In certain embodiments, the biomarkers comprise MUC-16 and SPINT1, and optionally further comprise any one or more of, preferably in the following order of addition, TACSTD2, CLEC6A, ICOSLG, MSMB, PROK1, WFDC2, KRT19 and FR-alpha. Thus, in a particular representative embodiment, the biomarkers comprise MUC-16, SPINT1, TACSTD2, CLEC6A, ICOSLG, MSMB, PROK1, WFDC2, KRT19 and FR-alpha.

In certain embodiments, the biomarkers comprise SPINT1 and MUC-16, and optionally further comprise any one or more of, preferably in the following order of addition, ICOSLG, CLEC6A, MSMB, TACSTD2, PROK1, WFDC2, KRT19 and FR-alpha. Thus, in a particular representative embodiment, the biomarkers comprise SPINT1, MUC-16, ICOSLG, CLEC6A, MSMB, TACSTD2, PROK1, WFDC2, KRT19 and FR-alpha.

In certain embodiments, the biomarkers comprise TACSTD2 and SPINT1 and optionally further comprise any one or more of, preferably in the following order of addition, MUC-16, CLEC6A, ICOSLG, MSMB, PROK1, WFDC2, KRT19 and FR-alpha. Thus, in a particular representative embodiment, the biomarkers comprise TACSTD1, SPINT1, MUC-16, CLEC6A, ICOSLG, MSMB, PROK1, WFDC2, KRT19 and FR-alpha.

In other embodiments the biomarker CDH3 may be included in the above lists.

The present invention provides in vitro methods of detecting, predicting or monitoring ovarian cancer in a subject. The present invention therefore provides methods to identify a subject having ovarian cancer, as well as to predict the likelihood of ovarian cancer in a subject (including the likelihood of a subject developing ovarian cancer), and/or to evaluate the progression of ovarian cancer and to monitor its treatment, by determining the level of biomarkers identified herein which have been found to be associated with ovarian cancer, in a sample from said subject.

In one embodiment, the present invention allows ovarian cancer to be detected in a subject. In other words, the present invention allows a subject having ovarian cancer, i.e. a subject who has established ovarian cancer, to be identified.

Detecting ovarian cancer in a subject may comprise determining in a sample from said subject the level of biomarkers associated with ovarian cancer which are disclosed herein, and comparing the level of said biomarkers with the level of the biomarkers determined in samples from a control group of patients who do not have cancer, or to a reference level, e.g. a reference level which has been predetermined to a be reference level for subjects who do or do not have ovarian cancer (or indeed the reference level may be for stage or type of ovarian cancer, or for risk of ovarian cancer etc). Changes in the level of the biomarkers in a subject as compared with a control group of patients (or put another way, differences between the level of the biomarkers in a subject as compared with a control group) may be indicative of that subject having ovarian cancer. In certain embodiments, this comparison may allow ovarian cancer to be detected.

In certain embodiments, the methods of the present invention may be used in a general population screen to detect or predict ovarian cancer in a subject. The methods of the present invention may therefore be used to detect or predict ovarian cancer in a subject who has not previously been diagnosed as having ovarian cancer. This would allow rapid, straightforward and cost-effective testing of subjects in the general population, and may allow subjects having ovarian cancer, or at risk of or having a predicted likelihood of developing ovarian cancer to be identified.

Subjects having ovarian cancer may frequently be asymptomatic, in particular at earlier stages of ovarian cancer progression. The methods of the present invention may be of particular utility in detecting ovarian cancer in a subject who is asymptomatic for ovarian cancer, as this may allow subjects who have ovarian cancer to be identified at an earlier stage, e.g. before the development of any symptoms. The methods of the present invention may therefore find utility as part of a general population screening programme to identify subjects within the general population who have ovarian cancer but who do not display any symptoms thereof. Beneficially, such screening may allow subjects who have ovarian cancer to be identified sooner, e.g. at an earlier stage of ovarian cancer, and thus may allow earlier treatment to be provided, as ovarian cancer which would not otherwise be undiagnosed. Accordingly, the methods of the present invention may be for detecting ovarian cancer in a subject who is asymptomatic for ovarian cancer.

In other embodiments, the subjects tested in screening methods may be subjects otherwise considered to be at a high risk for developing ovarian cancer, based on factors such as age, family history (e.g. comprising genetic testing such as BRCA-1 or BRCA-2), menopausal status and/or lifestyle factors such as weight, diet and/or smoking status.

In particular embodiments, the methods of the present invention may be used to identify particular subjects who are at a greater risk of developing ovarian cancer. Subjects identified in this way may optionally undergo additional regular longitudinal testing, in order to ensure that if a subject does develop ovarian cancer, said cancer is identified in a timely manner, and the risk of a subject developing ovarian cancer which would otherwise go untreated is reduced. In particular, such subjects may be referred for longitudinal testing e.g. by the methods of the present invention.

The present invention thus allows a subject at risk of developing ovarian cancer to be identified. Such methods comprise determining the level of biomarkers associated with ovarian cancer which are disclosed herein in a sample from said subject, and comparing the level of said biomarkers with the level of the biomarkers determined in a control group of patients who do not have cancer. Changes in the level of the biomarkers in a subject as compared with a control group of patients may be indicative of a subject being at risk of developing ovarian cancer.

In other embodiments, however, the subject may be symptomatic for ovarian cancer. Thus, according to one embodiment of this embodiment of the present invention, the method of identifying a subject having ovarian cancer may be performed on a subject presenting with symptoms of ovarian cancer. Symptoms for ovarian cancer include feeling bloated, a swollen abdomen, discomfort in the abdomen or pelvic area, loss of appetite, frequent urination, back pain, vaginal bleeding and fatigue. The present invention may allow identification of a subject who has ovarian cancer in a subject who displays one or more symptoms thereof.

Symptoms for ovarian cancer are typically shared with other gynaecological conditions, such as endometriosis, fibroids, polycystic ovary syndrome (PCOS), urinary tract infection (UTI) or benign gynaecological tumours which are not cancerous. Beneficially, the present invention may also allow a subject who has ovarian cancer to be identified from amongst subjects who may have similar symptoms, but who have a different gynaecological condition. Thus, the present invention in order to provide a differential diagnosis of ovarian cancer over one or other of these alternative possible conditions in a simple and non-invasive manner. Differential diagnosis of ovarian cancer (i.e. the identification of a subject who has ovarian cancer from amongst subjects with gynaecological conditions in general) is therefore particularly contemplated according to the methods of the present invention. In a particular embodiment, ovarian cancer may be distinguished from a benign ovarian tumour. In other words it may be determined whether a subject who has an ovarian tumour has ovarian cancer or a benign tumour (or put another way, the methods allow it to be determined whether an ovarian tumour in a subject is malignant or benign). Accordingly, the methods disclosed herein allow a benign and malignant tumours to be distinguished. A subject who is presenting with an adnexal ovarian mass (e.g. as determined by TVU) may be determined to have, or to be likely to have, ovarian cancer (or conversely, to have, to be likely to have, a benign ovarian tumour).

The present invention also provides methods for monitoring ovarian cancer, i.e. the progression of ovarian cancer or the effectiveness of therapy in subjects who have been diagnosed as having ovarian cancer. Such methods comprise determining the levels of biomarkers as identified herein at two or more time points in a subject who has been found to have ovarian cancer, and comparing the levels of the biomarkers in such a subject with a biomarker profile from a control group. Progression of ovarian cancer in a subject will result in the development of a biomarker profile over time which resembles a cancer profile (i.e. the biomarker profile will diverge from a non-cancer profile), whereas an effective therapy which reverses the progression of ovarian cancer will result in the development of a biomarker profile over time which resembles a non-cancer profile (i.e. the biomarker profile will diverge from a cancer profile).

In one embodiment, the present invention therefore provides methods for assessing the therapeutic efficacy of ovarian cancer treatment, said methods comprising comparing the biomarker profiles in samples taken from a subject before and after the treatment or during the course of treatment, wherein the biomarker profiles comprise biomarkers associated with ovarian cancer as defined elsewhere herein, wherein a change in the biomarker profile over time toward a non-cancer profile or to a stable profile or away from a cancer profile is interpreted as efficacy.

Conversely, the present invention also provides method for determining whether a subject is developing cancer, comprising: comparing the biomarker profiles in samples taken from a subject at two or more points in time, wherein the biomarker profiles comprise biomarkers associated with ovarian cancer as defined elsewhere herein, wherein a change in the biomarker profile toward a cancer profile or away from a non-cancer profile, is interpreted as a progression toward developing cancer.

According to such embodiments, a biomarker profile may be defined which is representative of a non-cancer profile (or a stable profile) based on determining the levels of biomarkers associated with ovarian cancer which are described herein in a control group. Conversely, a biomarker profile may be defined which is representative of a cancer profile based on determining the level of such biomarkers in a group of patients who have ovarian cancer. For the sake of such embodiments, however, changes in the determined biomarker profile toward a cancer profile or toward a non-cancer profile may not necessarily result in a biomarker profile which corresponds fully to a cancer profile or a non-cancer profile being achieved (i.e. a diagnostic threshold for such a profile may not be met). It may therefore be sufficient for the determined level of a biomarker simply to move towards the level seen in a cancer profile or a non-cancer.

The present invention is therefore directed to the diagnosis of ovarian cancer, and provides various measures of the status of ovarian cancer (or risk or likelihood thereof) in a subject, including on-going monitoring.

The biomarkers identified in the course of making the present invention may be markers specific for ovarian cancer. In particular, particular determined levels of the biomarkers discussed herein may provide a signature biomarker profile which is indicative of ovarian cancer, and allow its specific diagnosis over other conditions. The methods of the present invention therefore specifically allow the diagnosis of ovarian cancer.

In particular representative embodiments, the present invention allows a specific diagnosis of ovarian cancer to be obtained over similar or related conditions. For example, in certain embodiments, as briefly mentioned above, the present invention allows ovarian cancer (i.e. malignant ovarian cancer) to be diagnosed in a subject, in order to establish whether a subject has ovarian cancer or merely a benign (i.e. non-malignant) tumour. Such a diagnosis may be made based on the determined level of the biomarkers in a sample taken from a subject. The present invention therefore provides methods for detecting ovarian cancer in a subject, which comprise determining whether a subject has ovarian cancer or a benign tumour based on the determined level of the biomarkers.

In yet further representative embodiments, the methods of the present invention may allow the stage of ovarian cancer to be determined. This may be determined based on the determined level of the biomarkers, e.g. by comparison with profiles of biomarkers for different stages of ovarian cancer. The present invention therefore provides methods of determining the stage of ovarian cancer based on the determined level of the biomarkers. Ovarian cancer diagnosed by such methods may be early stage ovarian cancer, e.g. stage I or stage II, or may be late stage ovarian cancer, e.g. stage III or stage IV. For example a stage may be distinguished from other stages, or stage I/II may be distinguished from stage III/IV.

The methods of the present invention may also allow the type of ovarian cancer to be determined. Thus, in certain embodiments, the methods may allow the detection, prediction of monitoring the type of ovarian cancer in a subject. The type of ovarian cancer may be serous, endometriod, mucinous or clear cell tumours.

The methods of the invention rely on levels of particular biomarkers being determined in a subject sample. Defined levels of particular biomarkers may be interpreted as a diagnosis of ovarian cancer (e.g. indicative of a subject having ovarian cancer, or may provide a prediction of a risk of developing ovarian cancer, or monitoring the progression and/or treatment of ovarian cancer, as described above). The methods of the invention may therefore require the determined levels of biomarkers to be interpreted, in order to allow ovarian cancer in a subject to be detected, predicted or monitored.

In certain embodiments, levels for biomarkers may be determined in samples taken from subjects in a control group. A suitable control group of patients may be selected depending on the requirements of a subject and/or a clinician. For example, interpretation of the levels of the biomarkers that are determined in a subject sample may comprise comparing the determined level of a biomarker with the level of said biomarker in samples taken from subjects in a control group, and identifying whether there is a difference between the determined level of the biomarker, and the level of said biomarker in the control group. A comparison between the determined level of the biomarker in a subject sample and in samples taken from subjects in a control group may allow a diagnosis of ovarian cancer to be established.

For example, in a representative embodiment, a control group may be a healthy control group comprising subjects who do not have ovarian cancer. In a particular embodiment, subjects in such a control group may additionally not comprise any other condition, e.g. other gynaecological conditions such as those described above. Viewed another way, a profile of biomarkers may be established from such a control group, which represents a “non-cancer” profile; such a profile may be used as a baseline in a diagnostic test and differences between such a profile and the levels of biomarkers which are determined in a sample from said subject may allow a diagnosis of ovarian cancer to be established. Similarly, reference or control profiles may be established for other groups, such as those discussed above, e.g. for particular stage(s), or types, of ovarian cancer, or for benign ovarian or other gynaecological conditions.

In a more particular embodiment, a control group may comprise subjects who are symptomatic for ovarian cancer but do not have ovarian cancer. Such subjects may, for example, have another gynaecological condition, other than ovarian cancer, for example a benign tumour. Such a control group may allow ovarian cancer in particular to be detected in a subject (i.e. a specific diagnosis of ovarian cancer), and in particular subjects having another gynaecological condition may be used as a control group for detecting whether a subject has ovarian cancer or another gynaecological condition (e.g. a benign tumour); any differences in the level of biomarkers which are determined in a sample from a subject as compared with levels in samples from subjects in the control group would indicate that said subject has ovarian cancer as opposed to another gynaecological condition. Thus, in particular, this may allow ovarian cancer to be detected in a subject, over another gynaecological condition (e.g. a benign tumour) based on differences in the determined level of the biomarkers.

In yet other embodiments, however, the control group may comprise subjects known to have ovarian cancer, and more particularly may comprise subjects having any particular stage of ovarian cancer (stage I to stage IV) discussed above. In such embodiments, similarities between levels of biomarkers determined in a sample from a subject and levels of biomarkers in samples from subjects in a control group may be indicative of ovarian cancer. In particular, a comparison with the levels of biomarkers determined in subjects in control groups having different stages of ovarian cancer may allow the particular stage of ovarian cancer in a subject to be determined, i.e. the cancer may be stratified.

Alternatively, a series of control groups may be used for comparison purposes, in order to allow ovarian cancer to be diagnosed, each representing a different patient cohort having a different ovarian cancer status, and each providing a different “profile” of biomarkers representative of the subjects in each group. The level of biomarkers determined in a sample from a subject may be compared to these various different profiles in order to allow a diagnosis of ovarian cancer to be made. Suitable control groups in such embodiments may include a negative control group (i.e. a healthy control group comprising subjects who do not have ovarian cancer), which allows a “non-cancer” profile to be established as described elsewhere above. In certain embodiments, a control group comprising subjects who do not have ovarian cancer but who may have another gynaecological condition as described above may be used; this may allow a “benign tumour” profile to be established. Furthermore, a positive control group may be provided, comprising subjects who do have ovarian cancer, in order to provide a “cancer profile”. In a further representative embodiments, a number of different positive control groups may be provided, each comprising patients having a different stage and/or type of ovarian cancer, in order to provide a number of different “cancer profiles” representative of different stages and/or types of ovarian cancer. In such embodiments, diagnosis of ovarian cancer may be performed by comparing the determined levels of biomarkers in a sample from a subject with any such profiles, and establishing the profile which is closest to that of the biomarkers in the subject sample.

The levels of biomarkers which are determined in a sample from a subject who has ovarian cancer may, therefore, be compared with the levels of biomarkers determined in “control” samples, and similarity to or divergence from the levels in a sample from a subject and a suitable control sample may be used to detect, monitor or predict ovarian cancer. Where the determined level of a biomarker in a sample from a subject is different to the level of said biomarker in a control sample, the level in the sample from a subject may be higher or lower than the level in the control sample, depending on the particular biomarker in question. Thus, for example, the level of certain biomarkers may be found to be higher in a healthy control group than in subjects who have ovarian cancer; determining a low level of such biomarkers in a sample from a subject may therefore be indicative of ovarian cancer. Conversely, the level of other biomarkers may be found to be higher in subjects who have ovarian cancer than in a healthy control group; determining a high level of such biomarkers in a sample from a subject may therefore be indicative of ovarian cancer. The biomarkers recited herein are biomarkers which were found to have different levels in subjects who have ovarian cancer and subjects in a healthy control group in an initial discovery procedure, and the methods of the present invention encompasses determining increases and decreases in the levels of different biomarkers in order to detect, predict or monitor ovarian cancer according to the methods of the present invention.

Based on levels of biomarkers detected in various control or reference groups models may be constructed for use in detecting, diagnosing, predicting or monitoring ovarian cancer, as discussed. Thus, mathematical models may be constructed. Such models may be used in conjunction with the determinations of biomarker levels in subject samples to make the diagnosis or prediction, or to assess risk or therapy. Statistical and mathematical methods for preparing such models are well known in the art. In practice, algorithms may be used to analyse the data relating to levels in control, or reference or subject samples, and to generate models. The model may take the form of an algorithm that is used to evaluate or assess the determined biomarker levels, or biomarker profiles, from the subject.

Such models may take other variables into account. In other words the models may also incorporate other variables, or co-variates. Such variables may relate to other parameters of the subject, such as age, or lifestyle (e.g. smoking, alcohol consumption, etc) or clinical parameters etc. In particular, the subject's age may be included in the model.

The biomarkers of the present disclosure and invention are proteins. Whilst it is possible to detect a biomarker by detecting expression of a protein (e.g. by detecting RNA, particularly mRNA, or even by detecting DNA or epigenetic changes in DNA, e.g. DNA methylation), the biomarkers may conveniently be detected, according to the present disclosure and invention, at the protein level (namely polypeptide level), i.e. as proteins or polypeptides in the sample. It will be understood that the utility of a protein as a protein biomarker depends on detection of the protein in a sample. It will further be understood that a protein may be detected by detecting a fragment of the protein and not necessarily the whole intact protein.

It is thus particularly contemplated that the biomarkers described herein are proteins or fragments thereof, particularly measurable, or detectable, fragments thereof. Thus, according to certain embodiments, determining the level of a particular biomarker in a sample comprises determining the level of a particular protein or fragment thereof in a sample. Similarly, sets of reagents to determine levels of biomarkers in a sample particularly comprise reagents to determine levels of proteins or fragments thereof in a sample.

The sample according to the present disclosure and invention may be a sample of any body tissue or fluid, including but not restricted to ovarian tissue (i.e. it may be any clinical sample taken from a subject). Indeed, in some preferred embodiments, the sample is not of ovarian tissue. However, according to certain representative embodiments, detection of biomarkers in the methods of the invention (i.e. determining the level of biomarkers) is not performed in situ in an ovarian tissue sample (e.g. an ovarian cancer tissue sample), or not in a ovarian tissue sample in which tissue structure or architecture is preserved. Put another way, according to certain embodiments, detection of biomarkers does not take place directly in an ovarian tissue sample, i.e. the level of the biomarkers is not determined in situ in an ovarian tissue sample. More particularly according to such embodiments, detection of biomarkers is not performed in situ in a fixed and/or permeabilised ovarian tissue sample (such as a tissue slice). It is understood, however, that the present invention relates to the in vitro testing of samples from subjects, and thus reference to determining the level of a biomarker in situ in a sample from a subject does not include determining the level of said biomarker in vivo in a subject, but rather to determining the level of the biomarker in a tissue sample which has previously been taken from a subject, and in which the biomarker may be detected in its natural or native location or site in the tissue, e.g. in a sample as it is found in the sample in nature.

According to certain embodiments, however, the level of biomarkers in an ovarian cancer tissue sample may be determined using techniques e.g. such as those discussed below which do not comprise in situ detection.

In certain embodiments, the subject sample may be obtained or prepared from ovarian tissue samples such as a tissue biopsy, and may thus be a tissue lysate (e.g. a fine needle biopsy) or cells isolated from a tissue biopsy, or may be primary cell cultures e.g. derived from a tissue biopsy, or culture fluid (conditioned medium) in which such cells have been grown.

In certain embodiments, however, the sample does not comprise ovarian tissue, i.e. the level of biomarkers in an ovarian tissue sample is not determined. Thus the sample may be a non-ovarian sample, or more particularly the sample may not be an ovarian tissue sample. In a representative embodiment, the subject sample may be a bodily fluid, preferably blood or a blood fraction such as blood plasma or serum, or may be lymph, cerebrospinal fluid, ascites fluid, peritoneal fluid or urine. In representative examples, the sample is plasma or serum. Thus, according to a particularly representative embodiment, the present invention comprises determining the level of biomarkers in plasma or serum.

In certain embodiments, the methods of the present invention may be performed at two or more time points on a subject, separated by a time-scale of months, e.g. 1, 2, 3, 4, 5, 6 or 9 months, or years, e.g. 1, 2, 3, 4, 5 or 10 years. Testing may also performed regularly on an on-going and recurring basis, separated by any such periods of time. A subject may, therefore be tested for ovarian cancer two, three, four, five or more times according to the methods of the present invention. Changes in levels of biomarkers occurring over time may be considered when performing the methods of the present invention, and may be used to assist or establish a diagnosis of ovarian cancer.

Various methods of the invention may comprise the generation of a report for a doctor of the levels (e.g. relative levels) of the biomarkers. In certain embodiments, such a report may of use to a doctor in detecting, predicting or monitoring ovarian cancer in a subject, i.e. it may indicate the presence or absence or progression or treatment of ovarian cancer in a subject, or may include an indication of the risk to a subject of developing ovarian cancer, e.g. a stratified risk of ovarian cancer.

The methods of the invention may be used in conjunction with other diagnostic tests, such as an X-ray, transvaginal ultrasound, laparoscopic surgical investigation, or pelvic computerized tomography in order to detect, predict or monitor ovarian cancer in a subject. Such tests may be performed in advance of the diagnosis methods of the present invention, and thus the diagnosis methods of the present invention may be used in order to confirm a diagnosis of ovarian cancer, or to provide a more detailed or precise indication of the progression of ovarian cancer in a subject, and/or to confirm whether an identified cyst or tumour is benign or malignant. Alternatively, the diagnosis methods of the present invention may be used to provide a first indication that a subject has ovarian cancer (e.g. as part of a general population screening programme, as outlined above), and additional diagnostic tests may be performed subsequent to such methods in order to confirm a diagnosis of ovarian cancer. In either event, the methods of the present invention may be combined with such methods to provide a diagnosis of ovarian cancer.

The methods of the present invention are highly accurate for determining the presence of ovarian cancer. Advantageously, the methods of the present invention provide a sensitivity and/or specificity each at least 85% or higher, preferably at least 90% or 92%, and most preferably at least 95% or 97%. Embodiments of the invention include methods having a sensitivity of at least 85% and a specificity of at least 55%, 60%, 65%, 70%, 75%, 80%, 85% or 90%. Other embodiments of the method include methods having a specificity of at least 85% and a sensitivity of at least 55%, 60%, 65%, 70%, 75%, 80%, 85% or 90%.

The sensitivity and selectivity of embodiments of the invention may be determined for a population of subjects who have ovarian cancer as compared to a control group, e.g. a healthy control group or a control group comprising subjects symptomatic for ovarian cancer but do not have ovarian cancer. The sensitivity and selectivity of the present invention may be asserted relative to any such suitable control group.

Further, the detection or diagnosis of ovarian cancer according to the methods herein may be combined with treatment of the ovarian cancer. Thus, following detection or diagnosis of ovarian cancer in a subject, an anti-cancer therapy may be administered to the subject. Different treatments for ovarian cancer are known in the art, and any of these, alone or combination may be used. This may include, surgery, chemotherapy, radiotherapy and/or immunotherapy.

Biomarkers are detected in vitro in the methods of the present invention (i.e. the level of biomarkers may be determined in an in vitro assay) using any convenient detection assay. Detection therefore takes place on a sample from a subject, which sample may optionally be processed prior to detection in order to isolate biomarkers and/or make biomarkers available for detection, e.g. by particular assays or techniques.

In certain embodiments, biomarkers may be detected using an immunoassay. Immunoassays may be e.g. homogeneous immunoassays or heterogeneous immunoassays, and may involve, for example, determining the extent to which the a labelled biomarker displaces a biomarker bound to a reagent immobilised on a solid surface (competitive heterogeneous immunoassay) or the extent to which a biomarker may displace a labelled biomarker bound to a reagent immobilised on a solid surface (competitive heterogeneous immunoassay). Alternatively, a biomarker may itself be immobilised on a solid surface and detected using (directly or indirectly) labelled reagents (one-site, non-competitive immunoassays), or may be bound to a reagent immobilised on a solid surface, and detected using a (directly or indirectly) labelled reagent (a two-site, non-competitive immunoassay, or “sandwich” assay). Particular representative immunoassays suitable for the detection of a biomarker include an enzyme-linked immunosorbent assay (ELISA), cloned enzyme donor immunoassay (CEDIA), magnetic immunoassay, radioimmunoassay (RIA). According to certain embodiments, biomarkers are not detected using immunohistochemistry, e.g. immunohistochemical staining combined with microscopy e.g. bright field or dark field microscopy.

In particular or representative embodiments, detection of the biomarker comprises a proximity-based detection assay (a “proximity assay”). Proximity assays are well known in the art and widely described in the literature. Proximity assays based on pairs (or more) of proximity probes each comprising an binding partner capable of binding directly or indirectly to an analyte (e.g. via an intermediate analyte-binding antibody or other binding partner for the analyte) and a nucleic acid domain which interacts with the nucleic acid domain of the other proximity probe(s) to generate a detectable signal when the probes have been bound in proximity (e.g. when the partners of an interacting pair have interacted or bound together) have been developed and commercialised by Olink Bioscience and Olink Proteomics of Uppsala, Sweden. The interaction between the nucleic acid domains of proximity probes may comprise a nucleic acid ligation and/or extension reaction and may be detected by detecting a ligation and/or extension product. The nucleic acid domains themselves may interact (e.g. may be ligated together), or they may template the formation of a ligation and/or extension product from one or more added oligonucleotides.

Particular mention may be made of an in situ proximity ligation assay (PLA) which may be used to detect an interaction in situ in a cell or tissue sample. Such an assay has been developed and is marketed under the Duolink® brand name. Proximity assay are described in U.S. Pat. Nos. 6,878,515, 7,306,904, WO 2007/107743, WO/EP2012/051474 and WO2012/152942.

In a particular embodiment, the biomarkers may be detected using a proximity extension assay (PEA). PEA comprises the simultaneous binding of a pair of proximity probes to a biomarker in proximity. Upon binding of the pair of proximity probes to the biomarker, the nucleic acid domains are capable of interacting and forming a nucleic acid duplex, which may enable at least one of the nucleic acid domains to be extended from its 3′ end. This extension product forms a detectable nucleic acid detection product, optionally following amplification e.g. by PCR. Such an assay has been developed and commercialised by the Applicant. Exemplary PEA methods are described in greater detail in WO 2012/104261 and US 2015/0044674. The named Olink panels described herein allow the multiplexed detection of proteins by PEA, and an exemplary multiplexed detection assay comprising PEA is described in greater detail in the Examples below.

Biomarkers may be detected singly according to the methods described herein, or more preferably multiple biomarker may be detected simultaneously, i.e. detection may comprise multiplexed detection.

In particular representative embodiments, any of the methods of the present invention may be performed using a set of reagents as defined herein. Thus, any one of the methods of the invention may comprise detecting the level of biomarkers in a subject sample (in particular, any two or more biomarkers) using a set of reagents according to the present invention.

The present invention provides sets of reagents to determine the levels of combinations of biomarkers in a subject sample. Sets of reagents for determining the levels of any combination of two or more biomarkers described herein are provided.

In particular, sets of reagents to determine the levels of two or more biomarkers in a sample are provided, wherein the biomarkers are the biomarkers as defined according to any of the methods of the present invention. The biomarkers are as defined according to any embodiment of the methods of the present invention, and in particular, the biomarkers are not solely IL10 and KLK10, IL10 and MUC-16, KLK10 and MUC-16, MUC-16, IL 10 and KLK10, or MUC-16 and KRT19, as outlined above with respect to the methods of the present invention.

The sets of reagents of the present invention comprise two or more reagents to determine the level of two or more biomarkers which are as defined above. According to various embodiments of the present invention, sets of reagents corresponding to any of the combinations of biomarkers outlined above are provided. Put another way, sets of reagents defined herein may comprise reagents which allow the level of any of the combinations of biomarkers outlined above to be determined.

The biomarkers may be:
a) KLK10 and CPE;
b) PTK7 and ICOSLG;
c) SEZ6L2 and ICOSLG;
d) KRT19 and CLEC4D; or
e) IL10 and ICOSLG.

The set of reagents may comprise, for example, reagents to determine the level of any of the combinations of three biomarkers set forth in Table 2.

In more particular embodiments, the biomarkers may be MUC-16 and:
a) TACSTD2, and SPINT1, KRT19 or CLEC4D;
b) TACSTD2, and, ICOSLG, CLEC6A or PROK1;
c) SPINT1, and TACSTD2, ICOSLG or CLEC6A;
d) SPINT1, and ICOSLG, CLEC6A or MSMB;
e) KLK10, and SPINT1, CPE or CLEC6A;
f) PTK7, and SPINT1, CLEC6A or SPINK5;
g) SEZ6L2, and SPINT1, SPINK5 or CLEC6A;
h) KRT19, and SPINT1, ICOSLG or CLEC4D;
i) KRT19, and TACSTD2, SPINT1 or ICOSLG;
j) IL10, and SPINT1, SPINK5 or CLEC6A;
k) SPINT1, and FR-Alpha, TACSTD2 or WFDC2; or
l) TACSTD2, and FR-Alpha, SPINT1 or WFDC2.

Alternatively, the biomarkers may be MUC-16 and:
a) KLK10, and CPE, CLEC6A or TACSTD2;
b) PTK7, and ICOSLG, MSMB or TACSTD2;
c) SEZ6L2, and ICOSLG, CLEC6A or TACSTD2;
d) KRT19, and CLEC4D, ICOSLG or MSMB; or
e) IL10, and ICOSLG, TACSTD2 or CLEC6A.

More particularly, the set of reagents may comprise reagents to determine the levels of biomarkers in a sample, wherein said biomarkers are:
a) MUC-16, TACSTD2, and SPINT1;
b) MUC-16, TACSTD2, and, ICOSLG;
c) MUC-16, SPINT1, and ICOSLG;
d) MUC-16, SPINT1 and TACSTD2;
e) MUC-16, KLK10, and SPINT1;
f) MUC-16, PTK7, and SPINT1;
g) MUC-16, SEZ6L2, and SPINT1;
h) MUC-16, KRT19, and SPINT1;
i) MUC-16, KRT19, and TACSTD2;
j) MUC-16, IL10, and SPINT1;
k) MUC-16, SPINT1, and WFDC2;
l) MUC-16, TACSTD2, and WFDC2;
m) MUC-16, SPINT1, and FR-Alpha; or
n) MUC-16, TACSTD2, and FR-Alpha.

Alternatively, the three biomarkers may be:
a) MUC16, KLK10, and CPE;
b) MUC16, PTK7, and ICOSLG;
c) MUC16, SEZ6L2, and ICOSLG;
d) MUC16, KRT19, and CLEC4D; or
e) MUC16, IL10, and ICOSLG.
   In an aspect, the present invention may further a set of reagents for determining the level of three biomarkers in a sample from said subject, wherein said three biomarkers comprise a first biomarker selected from MUC-16, TACSTD2, and SPINT1 and second and third biomarkers selected from TACSTD2, KRT19, PTK7, SPINT1, MUC-16, KLK10, ICOSLG, IL10, SPINK5, CLEC6A, CLEC4D, CD83, SKAP1 and SEZ6L2, wherein the second and third biomarkers are different from the first biomarker, and from each other, and wherein the biomarkers are not solely MUC-16, IL10 and KLK10.

In particular, in an embodiment the reagents are not solely for determining any three or all four of the biomarkers MUC-16, IL10, KLK10 and KRT19.

In particular embodiments of this aspect, the three biomarkers may include at least one of TACSTD2, PTK7, SPINT1, ICOSLG, SPINK5, CLEC6A, CLEC4D, CD83, SKAP1 and SEZ6L2, or

at least one of PTK7, SPINT1, ICOSLG, SPINK5, CLEC6A, CLEC4D, CD83, SKAP1 and SEZ6L2, or

at least one of TACSTD2, PTK7, ICOSLG, SPINK5, CLEC6A, CLEC4D, CD83, SKAP1 and SEZ6L2, or

at least one of TACSTD2, PTK7, SPINT1, SPINK5, CLEC6A, CLEC4D, CD83, SKAP1 and SEZ6L2, or

at least one of PTK7, ICOSLG, SPINK5, CLEC6A, CLEC4D, CD83, SKAP1 and SEZ6L2, or

at least one of TACSTD2, PTK7, SPINK5, CLEC6A, CLEC4D, CD83, SKAP1 and SEZ6L2, or

at least one of PTK7, SPINT1, SPINK5, CLEC6A, CLEC4D, CD83, SKAP1 and SEZ6L2, or

at least one of PTK7, SPINK5, CLEC6A, CLEC4D, CD83, SKAP1 and SEZ6L2. In alternative embodiments, the set of reagents may comprise reagents to determine the levels of biomarkers in a sample, wherein said biomarkers are:
a) TACSTD2, MUC-16 and SPINT1, ICOSLG, KRT19, KLK10, CLEC6A or SEZ6L2;
b) TACSTD2, ICOSLG and CLEC6A or CLEC4D;
c) TACSTD2, SPINT1 and KRT19, MUC-16 or SPINK5;
d) SPINT1, MUC-16 and TACSTD2, ICOSLG, KRT19 or KLK10;
e) MUC-16, ICOSLG and TACSTD2, CLEC4D, CLEC6A, SPINT1 or SEZ6L2;
f) KRT19, SPINT1 and MUC-16, TACSTD2 or CD83;
g) TACSTD2, KRT19 and MUC-16, SPINT1 or KLK10;
h) TACSTD2, KLK10 and IL10, KRT19, MUC-16 or SKAP1;
i) IL10, MUC-16 and KRT19 or PTK7;
j) SPINT1, SPINK5 and PTK7, MO or SEZ6L2; or
k) TACSTD2, MO and SKAP1.

In further representative embodiments, the set of reagents may comprise reagents to determine the levels of biomarkers in a sample, wherein said biomarkers are:
a) TACSTD2, SPINT1 and MUC-16;
b) SPINT1, MUC-16 and ICOSLG;
c) SPINT1, KRT19 and MUC-16;
d) TACSTD2, SPINT1 and KRT19;
e) TACSTD2, MUC-16 and KRT19;
f) TACSTD2, MUC-16 and ICOSLG;
g) KRT19, MO and MUC-16;
h) SPINT1, MO and SPINK5;
i) MUC-16, TACSTD2 and CLEC6A;
j) TACSTD2, SPINT1 and SPINK5;
k) TACSTD2, KLK10 and KRT19;
l) SPINT1, KLK10 and MUC-16;
m) MUC-16, ICOSLG and CLEC6A;
n) TACSTD2, ICOSLG and CLEC6A;
o) TACSTD2, KLK10 and MO;
p) TACSTD2, MO and SKAP1;
q) MUC-16, ICOSLG and CLEC4D;
r) TACSTD2, ICOSLG and CLEC4D;
s) SPINT1, KRT10 and CD83;
t) TACSTD2, KLK10 and MUC-16;
u) SPINT1, PTK7 and SPINK5;
v) TACSTD2, KLK10 and SKAP1;
w) SEZ6L2, SPINT1 and SPINK5
x) TACSTD2, SEZ6L2 and MUC-16;
y) SEZ6L2, MUC-16 and ICOSLG; or
z) IL10, PTK7 and MUC-16.

More particularly, the set of reagents may comprise reagents to determine the levels of biomarkers in a sample, wherein said biomarkers are:
a) TACSTD2, SPINT1, KRT19 and CLEC4D;
b) TACSTD2, ICOSLG, CLEC6A and PROK1;
c) SPINT1, TACSTD2, ICOSLG and CLEC6A;
d) SPINT1, ICOSLG, CLEC6A and MSMB;
e) KLK10, SPINT1, CPE and CLEC6A;
f) PTK7, SPINT1, CLEC6A and SPINK5;
g) SEZ6L2, SPINT1, SPINK5 and CLEC6A;
h) KRT19, SPINT1, ICOSLG and CLEC4D;
i) KRT19, TACSTD2, SPINT1 and ICOSLG;
j) IL10, SPINT1, SPINK5 and CLEC6A;
k) SPINT1, FR-Alpha, TACSTD2 and WFDC2; or
l) TACSTD2, FR-Alpha, SPINT1 and WFDC2.

Optionally, said sets of reagents may further comprise a reagent to determine the level of MUC-16 in a sample.

Alternatively, the biomarkers may be:
a) KLK10, CPE, CLEC6A and TACSTD2;
b) PTK7, ICOSLG, MSMB and TACSTD2;
c) SEZ6L2, ICOSLG, CLEC6A and TACSTD2;
d) KRT19, CLEC4D, ICOSLG and MSMB; or
e) IL10, ICOSLG, TACSTD2 and CLEC6A.

Optionally, said sets of reagents may further comprise a reagent to determine the level of MUC-16 in a sample.

In further alternative embodiments, the set of reagents may comprise reagents to determine the levels of biomarkers in a sample, wherein said biomarkers are:
a) TACSTD2, SPINT1, KRT19 and CLEC4D;
b) TACSTD2, SPINT1, KRT19 and WFDC2;
c) TACSTD2, SPINT1, KRT19 and FR-Alpha;
d) TACSTD2, ICOSLG, CLEC6A and PROK1;
e) SPINT1, ICOSLG, CLEC6A and MSMB;
f) SPINT1, TACSTD2, ICOSLG and CLEC6A;
g) KLK10, SPINT1, CPE and CLEC6A;
h) PTK7, SPINT1, CLEC6A and SPINK5;
i) SEZ6L2, SPINT1, SPINK5 and CLEC6A;
j) KRT19, SPINT1, ICOSLG and CLEC4D;
k) KRT19, TACSTD2, SPINT1 and ICOSLG; or
l) IL10, SPINT1, SPINK5 and CLEC6A.

Optionally, said sets of reagents may further comprise a reagent to determine the level of MUC-16 in a sample.

Alternatively, the biomarkers may be:
a) KLK10, CPE, CLEC6A and TACSTD2;
b) PTK7, ICOSLG, MSMB and TACSTD2;
c) SEZ6L2, ICOSLG, MSMB and TACSTD2;
d) KRT19, CLEC4D, ICOSLG and MSMB; or
e) IL10, ICOSLG, TACSTD2 and CLEC6A.

Optionally, said sets of reagents may further comprise a reagent to determine the level of MUC-16 in a sample.

In certain embodiments, the set of reagents comprises reagents to determine the levels of MUC-16 and SPINT1, and optionally further comprises reagents to determine the level of any one or more of, preferably in the following order of addition, TACSTD2, CLEC6A, ICOSLG, MSMB, PROK1, WFDC2, KRT19 and FR-alpha. Thus, in a particular representative embodiment, the set of reagents comprises reagents to determine the levels of MUC-16, SPINT1, TACSTD2, CLEC6A, ICOSLG, MSMB, PROK1, WFDC2, KRT19 and FR-alpha in a sample.

In certain embodiments, the set of reagents comprises reagents to determine the levels of SPINT1 and MUC-16, and optionally further comprises reagents to determine the level of any one or more of, preferably in the following order of addition, ICOSLG, CLEC6A, MSMB, TACSTD2, PROK1, WFDC2, KRT19 and FR-alpha. Thus, in a particular representative embodiment, the set of reagents comprises reagents to determine the levels of SPINT1, MUC-16, ICOSLG, CLEC6A, MSMB, TACSTD2, PROK1, WFDC2, KRT19 and FR-alpha.

In certain embodiments, the set of reagents comprises reagents to determine the levels of TACSTD2 and SPINT1 and optionally further comprises reagents to determine the level of any one or more of, preferably in the following order of addition, MUC-16, CLEC6A, ICOSLG, MSMB, PROK1, WFDC2, KRT19 and FR-alpha. Thus, in a particular representative embodiment, the set of reagents comprises reagents to determine the levels of SPINT1, MUC-16, CLEC6A, ICOSLG, MSMB, PROK1, WFDC2, KRT19 and FR-alpha.

In certain embodiments, sets of reagents may be suitable for performing the methods of the present invention.

The reagents may be binding agents having the ability to bind to a biomarker as defined herein or to a fragment thereof. In particular the binding agent may bind specifically to a biomarker or to a fragment thereof. By binding specifically is meant that the binding agent is capable of binding to a biomarker in a manner which distinguishes it from binding to a non-target molecule. Thus, binding to a non-target molecule may be negligible or substantially reduced as compared to binding to its respective biomarker. A binding agent may thus be any agent having a binding affinity for its biomarker i.e. an affinity binding partner for a biomarker.

Reagents of the present invention may thus be selected from proteins or polypeptides such as antibodies or fragments or derivatives thereof, a combinatorially derived polypeptide from phage display or ribosome display or any other peptide display system, or a nucleic acid molecule, such as an aptamer, or combinations thereof.

In a preferred embodiment of the invention, the binding agent is a protein, preferably an antibody or derivative or fragment thereof. Various antibody-like molecules are also known and described in the art and may be used, e.g. affibodies and such like.

In a preferred embodiment, the binding agent is or comprises an antibody. The antibody may be of any convenient or desired species, class or sub-type. Furthermore, the antibody may be natural, derivatised or synthetic. The term “antibody” as used herein thus includes all types of antibody molecules and antibody fragments.

The term “antibody” as used herein can mean an antibody binding fragment or derivative or mimetic thereof, where these fragments, derivatives and mimetics possess the binding affinity for the target analyte. For example, antibody fragments, such as Fv, F(ab)2 and Fab may be prepared by cleavage of the intact protein, e.g. by protease or chemical cleavage. Also of interest are recombinantly or synthetically produced antibody fragments or derivatives, such as single chain antibodies or scFvs, or other antibody derivatives such as chimeric antibodies or CDR-grafted antibodies, where such recombinantly or synthetically produced antibody fragments retain the binding characteristics of the above antibodies, i.e. that they are capable of binding specifically to the target analyte. Such antibody fragments or derivatives generally include at least the VH and VL domains of the subject antibodies, so as to retain the binding characteristics of the subject antibodies. Such antibody fragments, derivatives or mimetics of the subject invention may be readily prepared using any convenient methodology, such as the methodology disclosed in U.S. Pat. Nos. 5,851,829 and 5,965,371; the disclosures of which are herein incorporated by reference. Thus, alternatively expressed, the binding agent may be, or may comprise, a binding protein comprising an antigen-binding domain obtained, derived from or based on antibody binding domain.

In particular representative embodiments, labels may be used to assist the detection of a biomarker in a sample. In an embodiment, the reagent may comprise a binding agent linked (e.g. conjugated to, or in any way associated with) with a label, or more generally a reporter group. The label, or reporter, may be any directly or indirectly signal-giving label or reporter. The label may be a protein or an enzyme, such as a fluorogenic or a chromogenic protein or enzyme (e.g. peroxidase or GFP, RFP, YFP of CFP), or alternatively may be a dye, e.g. a fluorescent dye such as fluorescein, rhodamine or mCherry, or a coloured dye or any other spectroscopically detectable label. In other embodiments, the label may be a radioactive isotope. In further embodiment, the label may be a nucleic acidor or oligonucleotide.

Thus, signal generating moieties may be provided as part of or conjugated to a reagent as described above, i.e. a reagent may directly comprise a signal generating moiety. Alternatively, reagents may be provided which do not comprise or which are not conjugated to a signal generating moiety, and a secondary binding agent, specific for a reagent as hereinbefore described, may be provided, which comprises or is conjugated to a signal generating moiety.

In certain embodiments, the reagents may be coupled to a nucleic acid tag, and detection of a biomarker may comprise detection of the nucleic acid tag, e.g. by quantitative real time PCR (qPCR). The “coupling” or connection as described above may be by any means known in the art, and which may be desired or convenient and may be direct or indirect e.g. via a linking group. For example, the domains (i.e. the binding domain and the nucleic acid domain) may be associated with one another by covalent linkage (e.g. chemical cross-linking) or by non-covalent association e.g., via streptavidin-biotin based coupling (biotin being provided on one domain and streptavidin on the other).

In yet further embodiments, the set of reagents may comprise a pair of reagents for each biomarker (e.g. a pair of binding agents, such as antibodies), each of which may comprise a nucleic acid domain coupled thereto, and which may bind simultaneously to their respective biomarker, or alternatively, the set of reagents may comprise a pair of secondary binding agents each of which may comprise a nucleic acid domain coupled thereto, and which are specific for a reagent or pair of reagents which bind to a biomarker, and may bind simultaneously to said reagent or pair of reagents. In such embodiments, the set of reagents may thus comprise for each biomarker a pair of proximity probes, wherein each proximity probe comprises a binding agent and a nucleic acid domain coupled thereto. The nucleic acid domains may comprise mutually complementary regions which are capable of interacting with one another when the pair of reagents are bound to a biomarker in proximity.

Sets of reagents according to the present invention may optionally provided fixed to a solid support, or may be provided in conjunction with a solid support and means for fixing the reagents thereto. Suitable such solid supports include particles (e.g. beads which may be magnetic or non-magnetic), sheets, gels, filters, membranes, fibres, capillaries, or microtitre strips, tubes, plates or wells etc.

The support may be made of glass, silica, latex or a polymeric material. Suitable are materials presenting a high surface area for binding of the analyte. Such supports may have an irregular surface and may be for example porous or particulate e.g. particles, fibres, webs, sinters or sieves. Particulate materials e.g. beads are useful due to their greater binding capacity, particularly polymeric beads.

Conveniently, a particulate solid support will comprise spherical beads. The size of the beads is not critical, but they may for example be of the order of diameter of at least 1 and preferably at least 2 μm, and have a maximum diameter of preferably not more than 10, and e.g. not more than 6 μm.

Monodisperse particles, that is those which are substantially uniform in size (e.g. size having a diameter standard deviation of less than 5%) have the advantage that they provide very uniform reproducibility of reaction. Representative monodisperse polymer particles may be produced by the technique described in U.S. Pat. No. 4,336,173.

According to a further aspect, the present invention provides a method of detecting biomarkers in a sample, said method comprising determining the level of two or more biomarkers in the sample, wherein the biomarkers are as defined herein. In particular, such a method may be performed on a subject sample, preferably wherein the subject has, or is suspected of having or is at risk of having ovarian cancer. Such methods may optionally be performed using sets of reagents as defined herein.

Sets of reagents according to the present invention may be provided in the form of test kits suitable for the detection of ovarian cancer biomarkers. Such test kits may optionally comprise additional reagents for the detection of biomarkers, such as one or more buffers, enzymes or detection reagents. Optionally said test kits may further comprise instructions for performing such detection or for performing an evaluation of biomarkers to predict the likelihood of ovarian cancer in a subject.

The reagents in a further embodiment are a multi-analyte panel assay comprising reagents to evaluate the expression levels of these biomarker panels.

### Embodiments

Various particular embodiments of the present disclosure are listed below:
1. An in vitro method of detecting, predicting or monitoring ovarian cancer in a subject, comprising determining in a sample from said subject the level of a biomarker selected from the list consisting of TACSTD2, SPINT1, PTK7, SEZ6L2, ICOSLG, SPINK5, CLEC6A, CLEC4D, SKAP1, CD83, PROK1, MSMB and CPE, wherein the level of the biomarker is not determined in situ in an ovarian tissue sample.
2. The method of embodiment 1, wherein said method comprises determining the level of two biomarkers in a sample from said subject, wherein said two biomarkers are:
   a. TACSTD2 and SPINT1;
   b. TACSTD2 and ICOSLG;
   c. TACSTD2 and KRT19;
   d. SPINT1 and ICOSLG;
   e. KLK10 and SPINT1;
   f. PTK7 and SPINT1;
   g. SEZ6L2 and SPINT1;
   h. KRT19 and SPINT1; or
   i. MO and SPINT1.
3. The method of embodiment 1, wherein the biomarkers comprise TACSTD2 or MUC-16 and one or more of ICOSLG, CLEC4D or CLEC6A.
4. The method of embodiment 3, wherein the biomarkers comprise (i) TACSTD2 or MUC-16, (ii) ICOSLG, and (iii) CLEC4D or CLEC6A.
5. An in vitro method of detecting, predicting or monitoring ovarian cancer in a subject, said method comprising determining in a sample from said subject the level of two biomarkers selected from the list consisting of TACSTD2, SPINT1, KLK10, KRT19, MUC-16, PTK7, SEZ6L and IL10 in a subject specimen, wherein
   (i) the biomarker levels determined are not solely of two biomarkers selected from the list consisting of IL10, KLK10 and MUC-16, and are not solely of MUC-16 and KRT19; and
   (ii) when the biomarkers are IL10 and KLK10, IL10 and MUC-16 or KLK10 and MUC-16, the level of at least one other biomarker is determined, wherein the at least one other biomarker is (a) from the list or (b) is not solely a kallikrein, CRP, EGF-R, CA-19-9, MIP-1 and/or ferritin;
   and
   (iii) when the biomarkers are MUC-16 and KRT19, the level of at least one other biomarker is determined, wherein the at least one other biomarker is (a) from the list or (b) is not solely (i) FasL and/or M-CSF; (ii) kallikrein 8, CEA and/or M-CSF; (iii) CEA, M-CSF and/or EGFR; or (iv) ErbB2, wherein the level of the biomarkers are not determined in situ in an ovarian tissue sample.
6. The method of embodiment 5, wherein when the biomarkers are IL10 and KLK10, IL10 and MUC-16, KLK10 and MUC-16, or KRT19 and MUC-16, or optionally when the biomarkers are any two, any three or all four of IL10, KLK10, MUC-16 and KRT19, the level of at least one other biomarker is determined, wherein the at least one other biomarker is selected from TACSTD2, PTK7, SEZ6L, SPINT1, CD83, CD99L2, CLEC4D, CLEC6A, CLMP, CPE, ICOSLG, IRF9, LAMP3, MSMB, PROK1, PTK7, SIT1, SKAP1, SPINK5 and TANK, or from PTK7, SEZ6L, TACSTD2, CD83, CD99L2, CLEC4D, CLEC6A, CLMP, CPE, IRF9, LAMP3, MSMB, PROK1, PTK7, SIT1, SKAP1, SPINK5 and TANK.
7. The method of embodiment 5 or 6, wherein the biomarkers comprise MUC-16 and one of TACSTD2, IL10, KLK10, KRT19, PTK7, SEZ6L and SPINT1.
8. The method of any one of embodiments 5 to 7, further comprising determining the level of at least one additional biomarker, preferably at least two or at least three additional biomarkers, selected from the list consisting of TACSTD2, CD83, CD99L2, CLEC4D, CLEC6A, CLMP, CPE, FR-alpha, ICOSLG, IL10, IRF9, KLK10, KRT19 LAMP3, MSMB, MUC-16, PROK1, PTK7, SEZ6L2, SIT1, SKAP1, SPINK5, SPINT1, TANK and WFDC2.
9. The method of any one of embodiments 5 to 8, wherein said biomarkers are MUC-16 and:
   a) TACSTD2, and SPINT1, KRT19 or CLEC4D;
   b) TACSTD2, and, ICOSLG, CLEC6A or PROK1;
   c) TACSTD2, and FR-Alpha, SPINT1 or WFDC2;
   d) SPINT1, and TACSTD2, ICOSLG or CLEC6A;
   e) SPINT1, and ICOSLG, CLEC6A or MSMB;
   f) KLK10, and SPINT1, CPE or CLEC6A;
   g) PTK7, and SPINT1, CLEC6A or SPINK5;
   h) SEZ6L2, and SPINT1, SPINK5 or CLEC6A;
   i) KRT19, and SPINT1, ICOSLG or CLEC4D;
   j) KRT19, and TACSTD2, SPINT1 or ICOSLG;
   k) IL10, and SPINT1, SPINK5 or CLEC6A; or
   l) SPINT1, and FR-Alpha, TACSTD2 or WFDC2.
10. The method of embodiment 9, wherein the levels of following sets of biomarkers are determined:
   a) MUC-16, TACSTD2, and SPINT1;
   b) MUC-16, TACSTD2, and, ICOSLG;
   c) MUC-16, TACSTD2, and KRT19;
   d) MUC-16, TACSTD2, and WFDC2;
   e) MUC-16, TACSTD2, and FR-Alpha
   f) MUC-16, SPINT1, and ICOSLG;
   g) MUC-16, KLK10, and SPINT1;
   h) MUC-16, PTK7, and SPINT1;
   i) MUC-16, SEZ6L2, and SPINT1;
   j) MUC-16, KRT19, and SPINT1;
   k) MUC-16, IL10, and SPINT1;
   l) MUC-16, SPINT1, and WFDC2; or
   m) MUC-16, SPINT1, and FR-Alpha.
11. The method of any one of embodiments 1 or 5 to 10, wherein said biomarkers are MUC-16 and:
   a) TACSTD2, SPINT1, KRT19 and CLEC4D;
   b) TACSTD2, SPINT1, KRT19 and WFDC2;
   c) TACSTD2, SPINT1, KRT19 and FR-Alpha;
   d) TACSTD2, ICOSLG, CLEC6A and PROK1;
   e) TACSTD2, SPINT1, ICOSLG and CLEC6A;
   f) TACSTD2, KRT19, SPINT1 and ICOSLG;
   g) SPINT1, ICOSLG, CLEC6A and MSMB;
   h) KLK10, SPINT1, CPE and CLEC6A;
   i) PTK7, SPINT1, CLEC6A and SPINK5;
   j) SEZ6L2, SPINT1, SPINK5 and CLEC6A;
   k) KRT19, SPINT1, ICOSLG and CLEC4D; or
   l) IL10, SPINT1, SPINK5 and CLEC6A.
12. An in vitro method of detecting, predicting or monitoring ovarian cancer in a subject, comprising determining the level of three biomarkers in a sample from said subject, wherein said three biomarkers comprise a first biomarker selected from TACSTD2, SPINT1 and MUC-16, and second and third biomarkers selected from SPINT1, TACSTD2, KRT19, PTK7, MUC-16, KLK10, ICOSLG, IL10, SPINK5, CLEC6A, CLEC4D, CD83, SKAP1 and SEZ6L2, wherein the second and third biomarkers are different from the first biomarker, and from each other, and wherein the biomarker levels determined are not solely of the biomarkers MUC-16, IL10 and KLK10, wherein the level of the biomarker is not determined in situ in an ovarian tissue sample.
13. The method of embodiment 12, wherein the three biomarkers are:
   a) TACSTD2, MUC-16 and SPINT1, ICOSLG, KRT19, KLK10, CLEC6A or SEZ6L2;
   b) TACSTD2, ICOSLG and CLEC6A or CLEC4D;
   c) TACSTD2, SPINT1 and KRT19, MUC-16 or SPINK5;
   d) TACSTD2, KRT19 and MUC-16, SPINT1 or KLK10;
   e) TACSTD2, KLK10 and IL10, KRT19, MUC-16 or SKAP1;
   f) TACSTD2, IL10 and SKAP1;
   g) SPINT1, MUC-16 and TACSTD2, ICOSLG, KRT19 or KLK10;
   h) MUC-16, ICOSLG and TACSTD2, CLEC4D, CLEC6A, SPINT1 or SEZ6L2;
   i) KRT19, SPINT1 and MUC-16, TACSTD2 or CD83;
   j) IL10, MUC-16 and KRT19 or PTK7; or
   k) SPINT1, SPINK5 and PTK7, MO or SEZ6L2.
14. The method of embodiment 12 or 13, wherein the three biomarkers are:
   a) TACSTD2, SPINT1 and MUC-16;
   b) TACSTD2, SPINT1 and KRT19;
   c) TACSTD2, MUC-16 and KRT19;
   d) TACSTD2, MUC-16 and ICOSLG;
   e) TACSTD2, MUC-16 and CLEC6A;
   f) TACSTD2, SPINT1 and SPINK5;
   g) TACSTD2, KLK10 and KRT19;
   h) TACSTD2, ICOSLG and CLEC6A;
   i) TACSTD2, KLK10 and MO;
   j) TACSTD2, IL10 and SKAP1;
   k) TACSTD2, ICOSLG and CLEC4D;
   l) TACSTD2, KLK10 and MUC-16;
   m) TACSTD2, KLK10 and SKAP1;
   n) TACSTD2, SEZ6L2 and MUC-16;
   o) SPINT1, MUC-16 and ICOSLG;
   p) SPINT1, KRT19 and MUC-16;
   q) KRT19, MO and MUC-16;
   r) SPINT1, MO and SPINK5;
   s) SPINT1, KLK10 and MUC-16;
   t) MUC-16, ICOSLG and CLEC6A;
   u) MUC-16, ICOSLG and CLEC4D;
   v) SPINT1, KRT10 and CD83;
   w) SPINT1, PTK7 and SPINK5;
   x) SEZ6L2, SPINT1 and SPINK5
   y) SEZ6L2, MUC-16 and ICOSLG; or
   z) IL10, PTK7 and MUC-16.
15. Use of TACSTD2, SPINT1, PTK7, SEZ6L2, ICOSLG, SPINK5, CLEC6A, CLEC4D, SKAP1, CD83, PROK1, MSMB or CPE as a biomarker for ovarian cancer.
16. A set of reagents to determine the levels of biomarkers in a sample, wherein the biomarkers are:
   a. TACSTD2 and SPINT1;
   b. TACSTD2 and ICOSLG;
   c. TACSTD2 and KRT19
   d. SPINT1 and ICOSLG;
   e. KLK10 and SPINT1;
   f. PTK7 and SPINT1;
   g. SEZ6L2 and SPINT1;
   h. KRT19 and SPINT1; or
   i) IL10 and SPINT1.
17. A set of reagents to determine the levels of biomarkers in a sample, wherein the biomarkers comprise a first biomarker selected from TACSTD2, SPINT1 and MUC-16, and second and third biomarkers selected from SPINT1, TACSTD2, KRT19, PTK7, MUC-16, KLK10, ICOSLG, IL10, SPINK5, CLEC6A, CLEC4D, CD83, SKAP1 and SEZ6L2, wherein the second and third biomarkers are different from the first biomarker and from each other, and wherein the biomarkers are not solely MUC-16, IL10 and KLK10.
18. The set of reagents of embodiment 17, wherein the three biomarkers are:
   a) TACSTD2, MUC-16 and SPINT1, ICOSLG, KRT19, KLK10, CLEC6A or SEZ6L2;
   b) TACSTD2, ICOSLG and CLEC6A or CLEC4D;
   c) TACSTD2, SPINT1 and KRT19, MUC-16 or SPINK5;
   d) TACSTD2, KRT19 and MUC-16, SPINT1 or KLK10;
   e) TACSTD2, KLK10 and IL10, KRT19, MUC-16 or SKAP1;
   f) TACSTD2, IL10 and SKAP1;
   g) SPINT1, MUC-16 and TACSTD2, ICOSLG, KRT19 or KLK10;
   h) MUC-16, ICOSLG and TACSTD2, CLEC4D, CLEC6A, SPINT1 or SEZ6L2;
   i) KRT19, SPINT1 and MUC-16, TACSTD2 or CD83;
   j) IL10, MUC-16 and KRT19 or PTK7; or
   k) SPINT1, SPINK5 and PTK7, MO or SEZ6L2.
19. The set of reagents of embodiments 17 or 18, wherein the three biomarkers are:
   a) TACSTD2, SPINT1 and MUC-16;
   b) TACSTD2, SPINT1 and KRT19;
   c) TACSTD2, MUC-16 and KRT19;
   d) TACSTD2, MYC-16 and ICOSLG;
   e) TACSTD2, MUC-16, TACSTD2 and CLEC6A;
   f) TACSTD2, SPINT1 and SPINK5;
   g) TACSTD2, KLK10 and KRD19;
   h) TACSTD2, ICOSLG and CLEC6A;
   i) TACSTD2, KLK10 and MO;
   j) TACSTD2, IL10 and SKAP1;
   k) TACSTD2, ICOSLG and CLEC4D;
   l) TACSTD2, KLK10 and MUC-16;
   m) TACSTD2, KLK10 and SKAP1;
   n) TACSTD2, SEZ6L2 and MUC-16;
   o) SPINT1, MUC-16 and ICOSLG;
   p) SPINT1, KRT19 and MUC-16;
   q) KRT19, MO and MUC-16;
   s) SPINT1, KLK10 and MUC-16;
   t) MUC-16, ICOSLG and CLEC6A;
   u) MUC-16, ICOSLG and CLEC4D;
   v) SPINT1, KRT10 and CD83;
   w) SPINT1, PTK7 and SPINK5;
   x) SEZ6L2, SPINT1 and SPINK5
   y) SEZ6L2, MUC-16 and ICOSLG; or
   z) IL10, PTK7 and MUC-16.
20. The set of reagents of any one of embodiments 16 to 19, wherein said reagents are binding agents capable of binding specifically to a said biomarker or to a fragment thereof, preferably wherein the binding agents are or comprise antibodies, and/or wherein the set of reagents comprises a pair of proximity probes for each biomarker, wherein each proximity probe comprises a binding agent and a nucleic acid domain conjugated thereto, and wherein each pair of proximity probes can bind simultaneously to their respective biomarker.
21. The method of any one of embodiments 1 to 14, wherein:
   A) said method is a method of identifying a subject having ovarian cancer, wherein a change in the levels of the biomarkers, as compared with a control group of patients who do not have cancer, is indicative of ovarian cancer;
   B) said method is a method of identifying a subject at risk of developing ovarian cancer, wherein a change in the levels of the biomarkers, as compared with a control group of patients who do not have cancer, is indicative of a risk of developing ovarian cancer;
   C) said method is a method of assessing the efficacy of ovarian cancer treatment and comprises comparing the biomarker profiles in samples taken from a subject before and after the treatment or during the course of treatment, wherein a change in the biomarker profile over time toward a non-cancer profile or to a stable profile is interpreted as efficacy; or
   D) said method is a method of determining whether a subject potentially is developing cancer and comprises comparing the biomarker profiles in samples taken from a subject at two or more points in time, wherein a change in the biomarker profile toward a cancer profile, is interpreted as a progression toward developing cancer.
22. The method of any one of embodiments 1 to 14 or 21, wherein the sample is a sample from a subject who has, is suspected of having, or is at risk of having or developing ovarian cancer, optionally wherein the subject has symptoms of ovarian cancer.
23. The method of any one of embodiments 1 to 14, 21 or 22, wherein the sample is a sample of body fluid or tissue other than ovarian tissue.
24. The method of any one of embodiments 1 to 14 or 21 to 23, wherein the sample is a sample of body fluid, preferably wherein the sample is blood or a blood-derived sample.
25. The method of any one of embodiments 1 to 14 or 21 to 24, comprising determining whether an ovarian tumour in a subject is malignant or benign, and/or determining the stage of ovarian cancer based on the determined level of the biomarkers, and/or wherein the ovarian cancer is stage I or stage II ovarian cancer, or stage III or stage IV ovarian cancer.

The present invention may be more fully understood with reference to the following non-limiting Figures and Examples, in which:

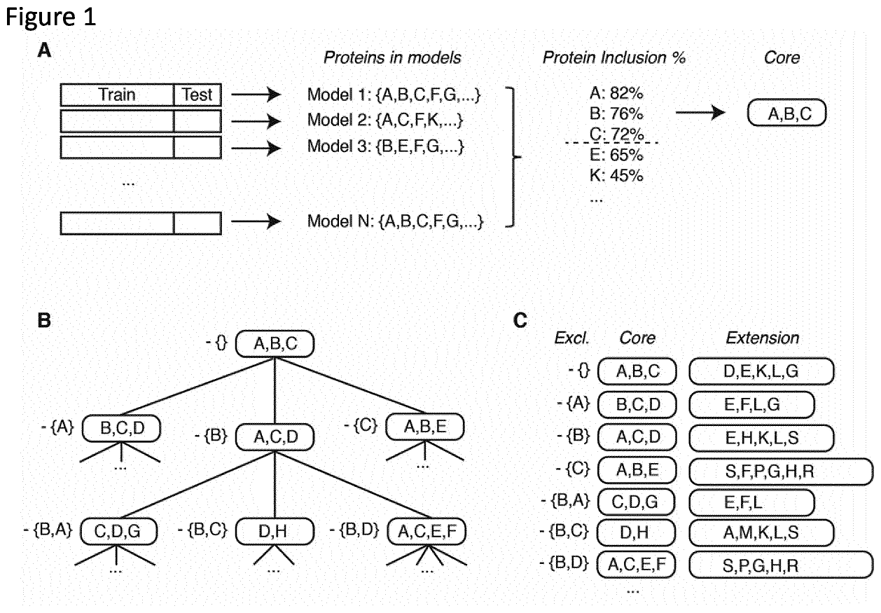

FIG. 1 shows the discovery process for identifying biomarkers and biomarker panels associated with ovarian cancer (A) Repeated model generation over random splits of the data. Proteins present in a sufficient fraction of the models are included into the core. (B) Generation of mutually exclusive cores. Proteins present in the first core (top node) are sequentially withheld from the second round of core discovery, as indicated by the sets to the left of the nodes. Each core of size N generates N new search-branches. (C) The final models are built by adding proteins to each core. The added proteins are chosen with respect to the proteins excluded in the core-discovery. Proteins are added in a stepwise forward selection choosing the protein that explains the highest proportion of remaining variance in the decision.

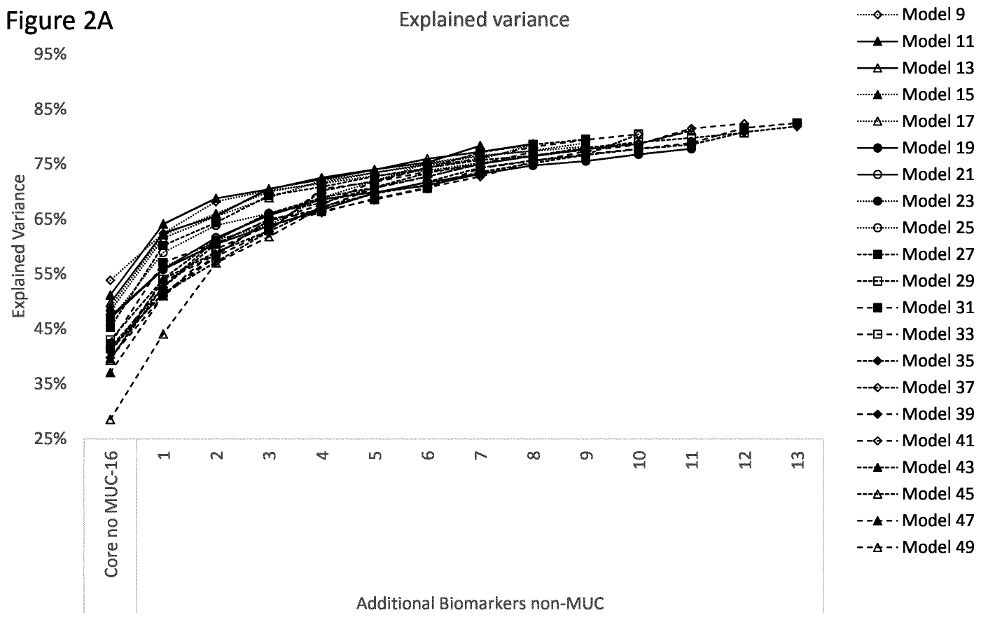

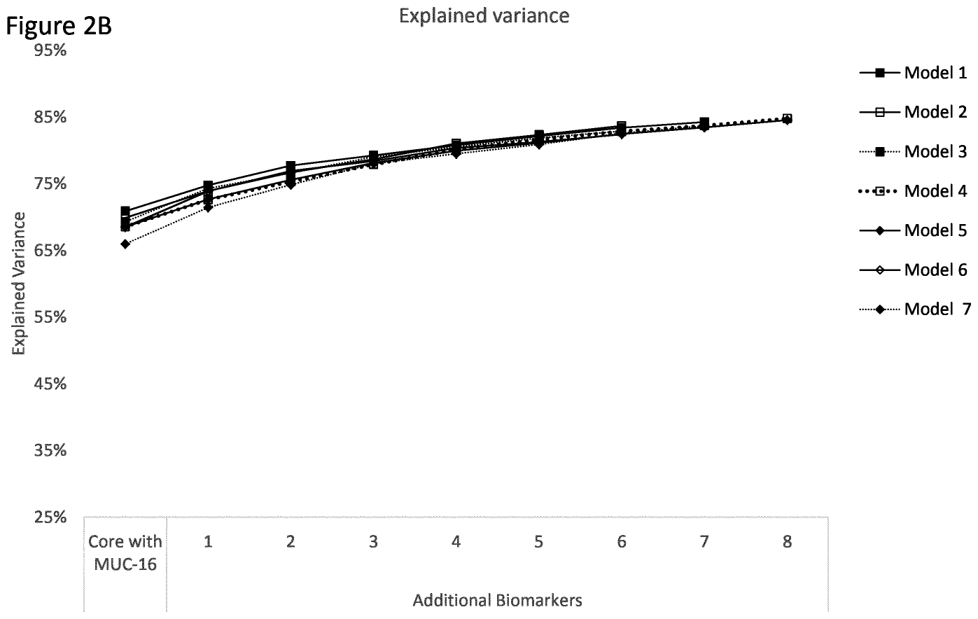

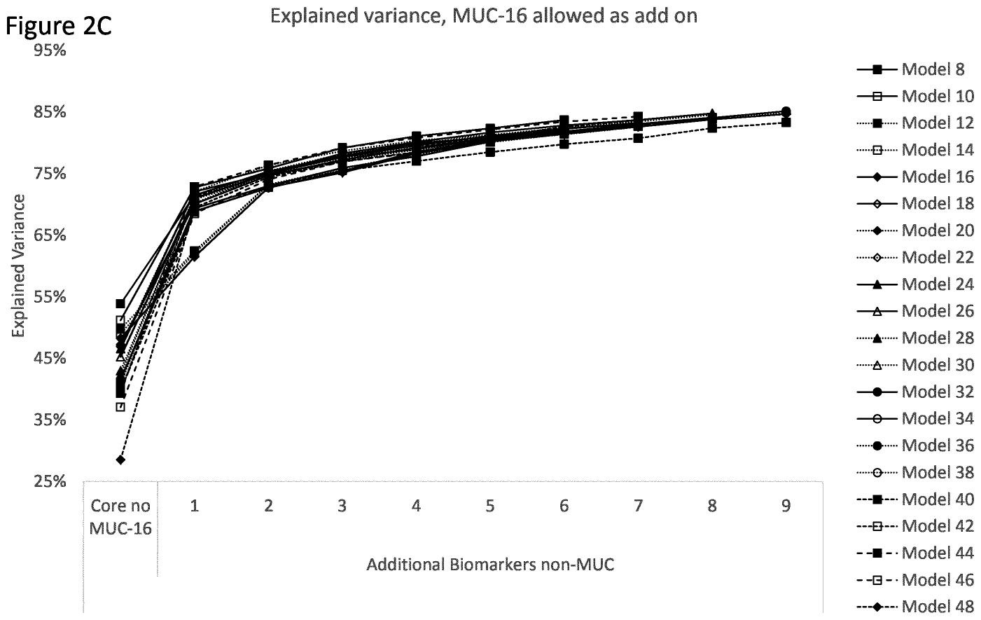

FIG. 2 shows the performance of 49 profiles developed using the biomarkers which were identified as being associated with ovarian cancer. A) Profiles which did not include MUC-16 as the first or second biomarker, or as an add-on. B) Profiles which included MUC-16 as the first or second biomarker. C) Profiles which did not include MUC-16 as the first or second biomarker, but which allowed MUC-16 to be included as a subsequent biomarker.

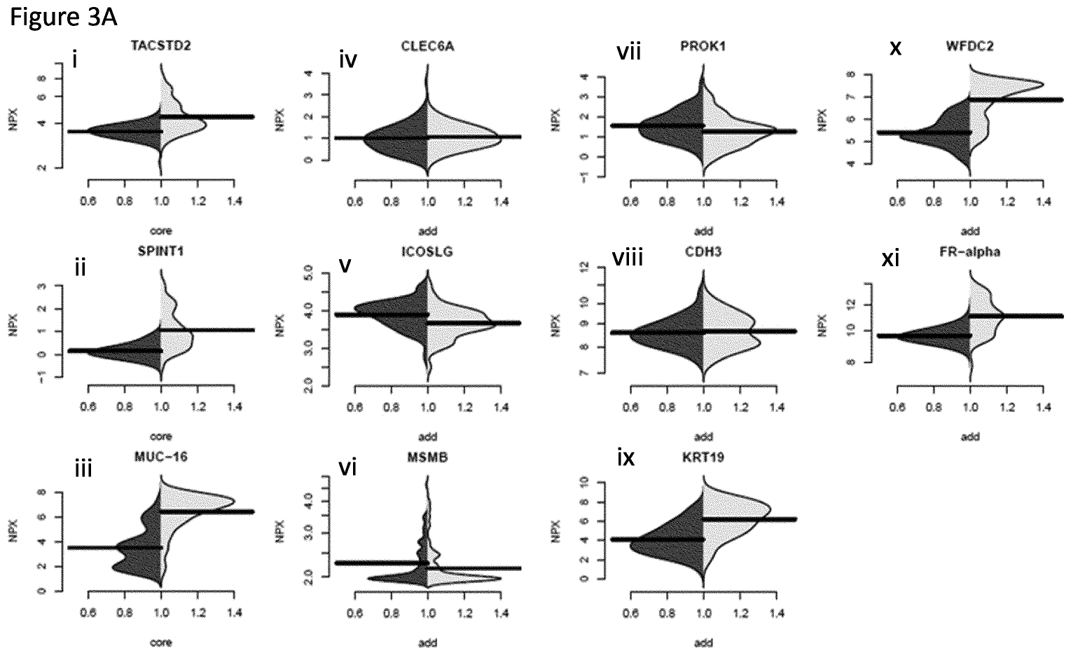

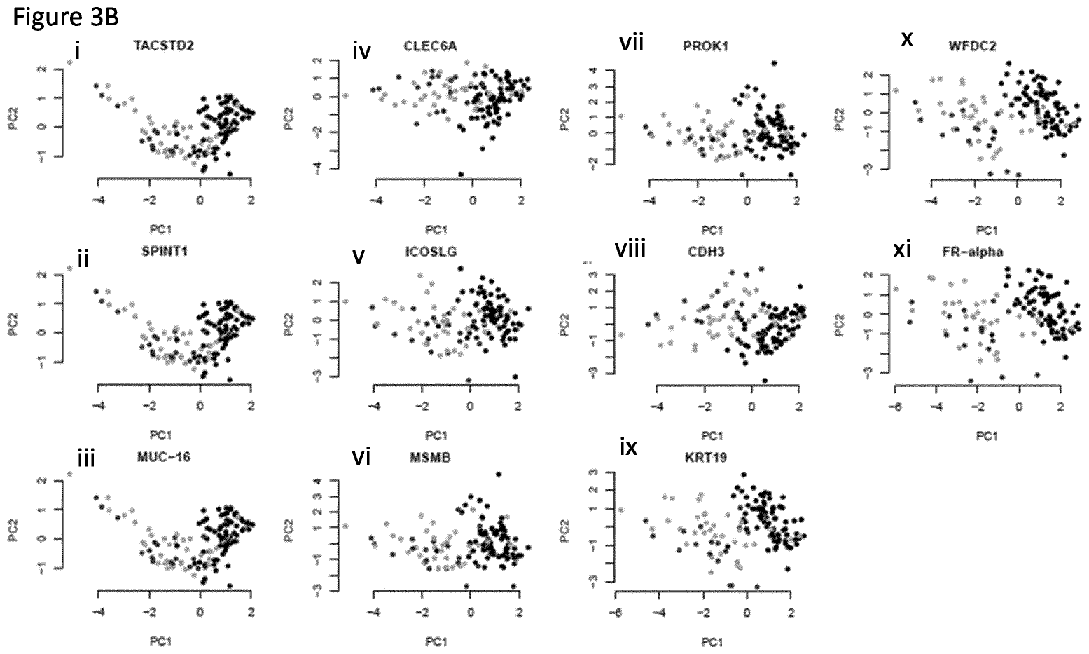

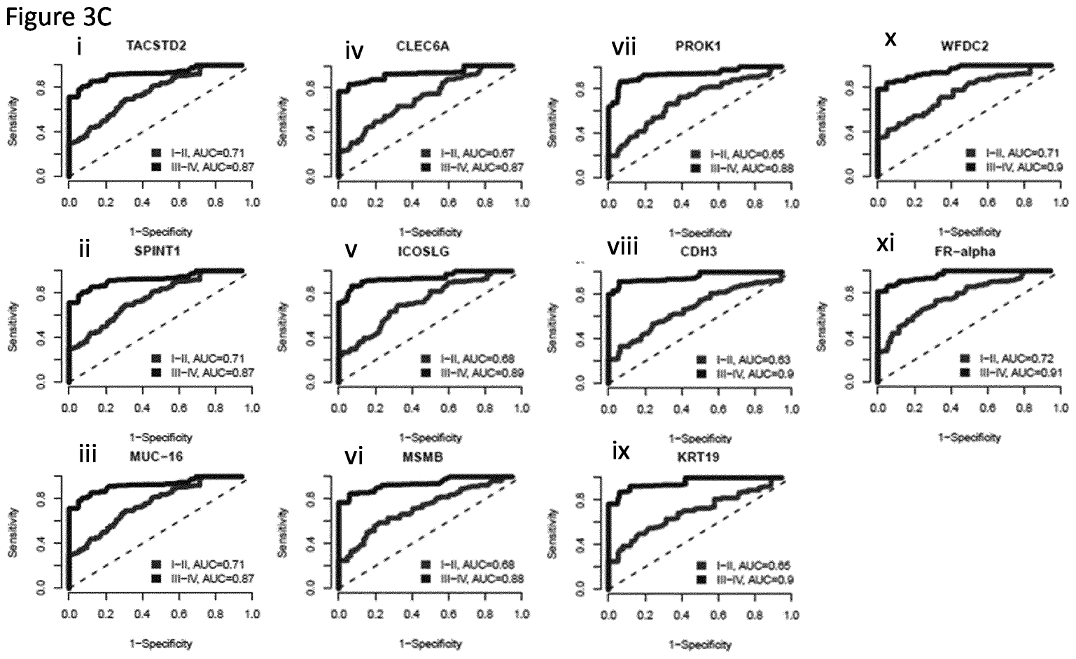

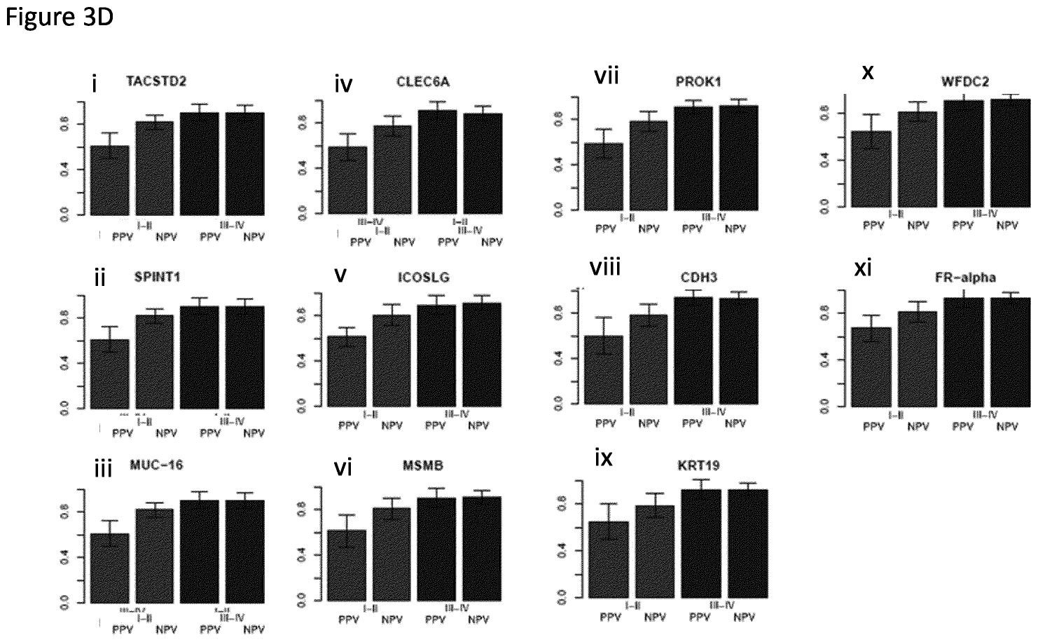

FIG. 3 shows the performance of a model comprising (i) TACSTD2, (ii) SPINT1, (iii) MUC-16, (iv) CLEC6A, (v) ICOSLG, (vi) MSMB, (vii) PROK1, (viii) CDH3, (ix) KRT19, (x) WFDC2 and (xi) FR-alpha. A) Abundance levels (density plots) of biomarkers in subjects with benign tumours (left) and ovarian cancer (right). B) Principle Component Analysis (PCA) after each protein was added after the core (TACSTD2, SPINT1 and MUC-16). C) ROC curves. D) PPV and NPV data for stage I-II and stage III-IV ovarian cancer. PCA, ROC curves and PPV and NPV data for stage I-II and stage III-IV ovarian cancer were re-calculated for each successive biomarker (values for TACSTD2, SPINT1 and MUC-16 were calculated using the same data).

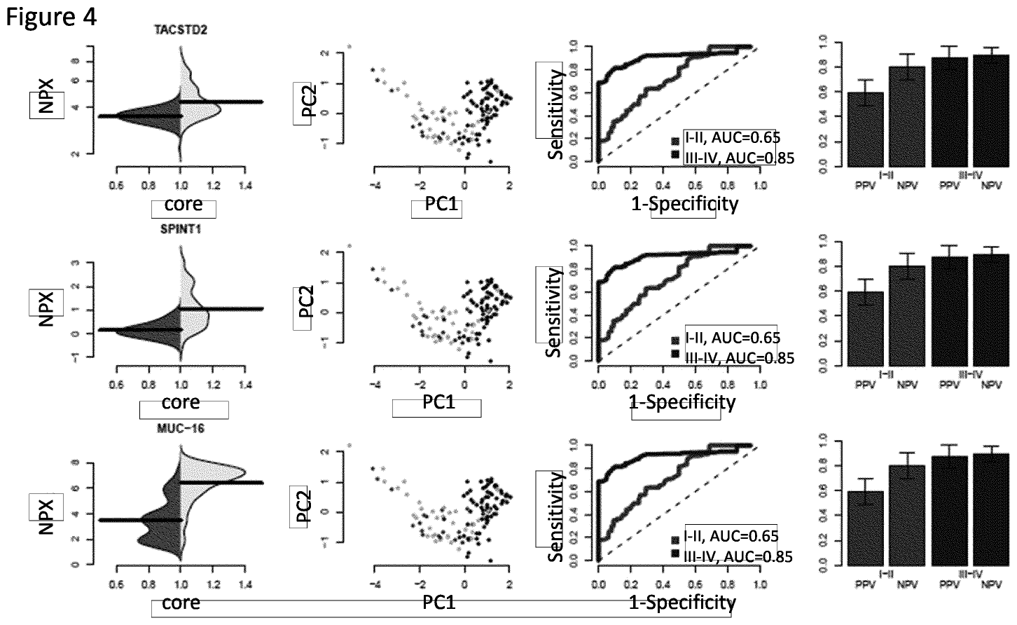

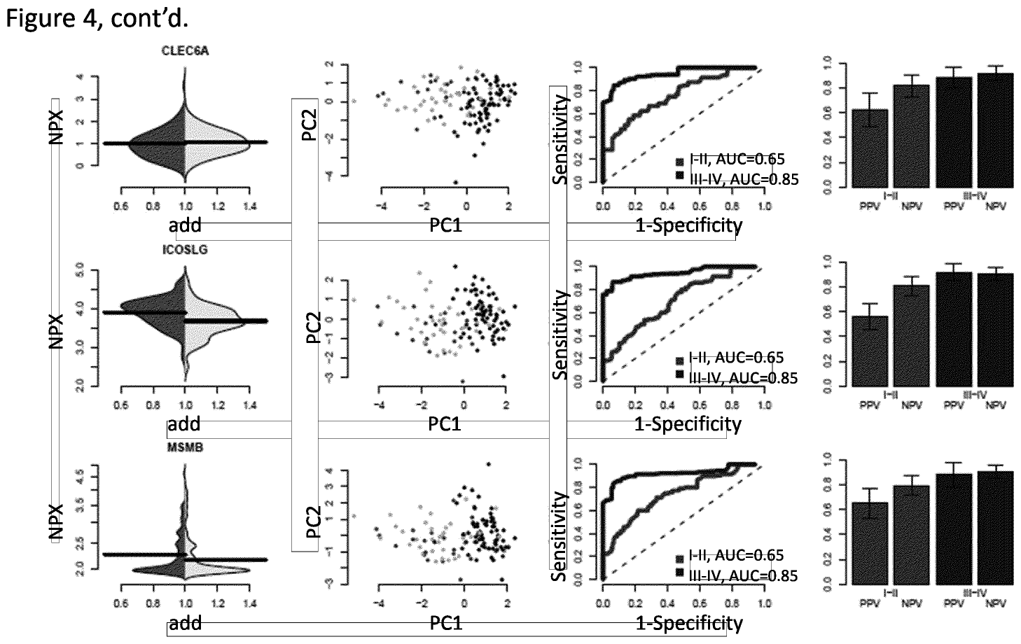

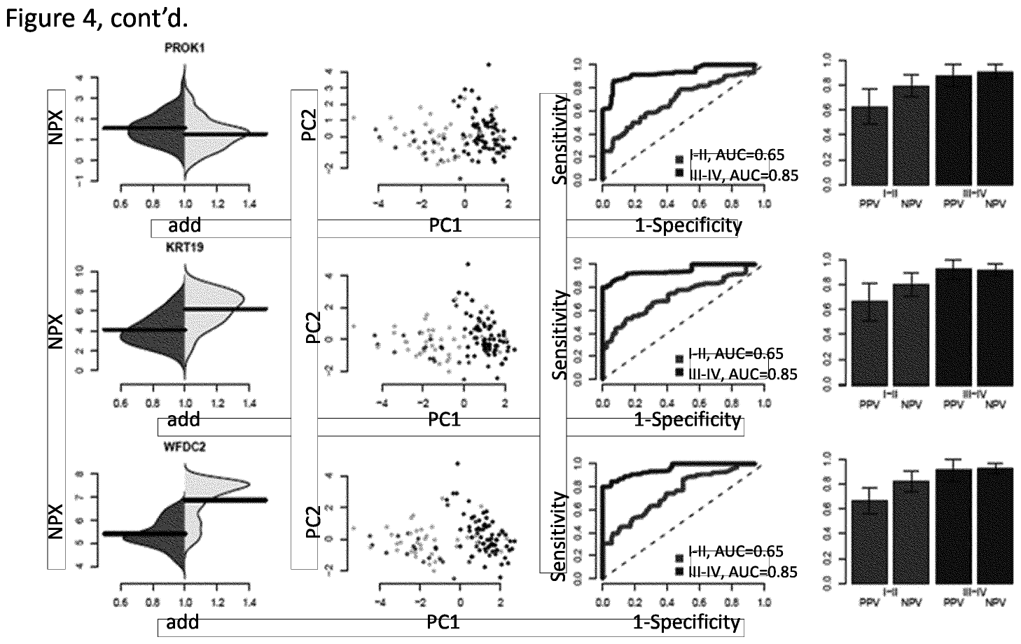

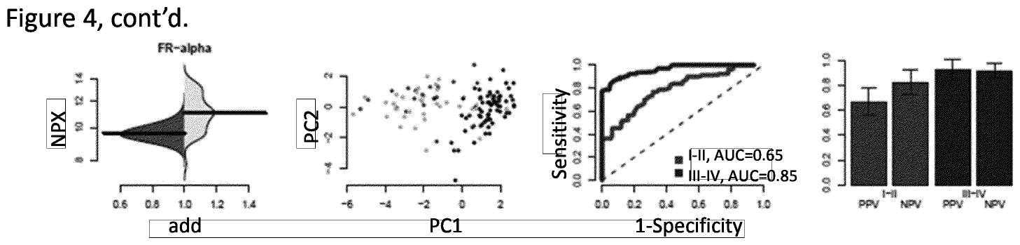

FIG. 4 shows the performance of a model (model 10) supplemented with KRT19, WFDC2 and FR-alpha. Abundance levels of biomarkers in subjects with benign tumours (left) and ovarian cancer (right) are shown. PCA, ROC curves and PPV and NPV data for stage I-II and stage III-IV ovarian cancer were re-calculated for each successive biomarker (values for TACSTD2, SPINT1 and MUC-16 were calculated using the same data).

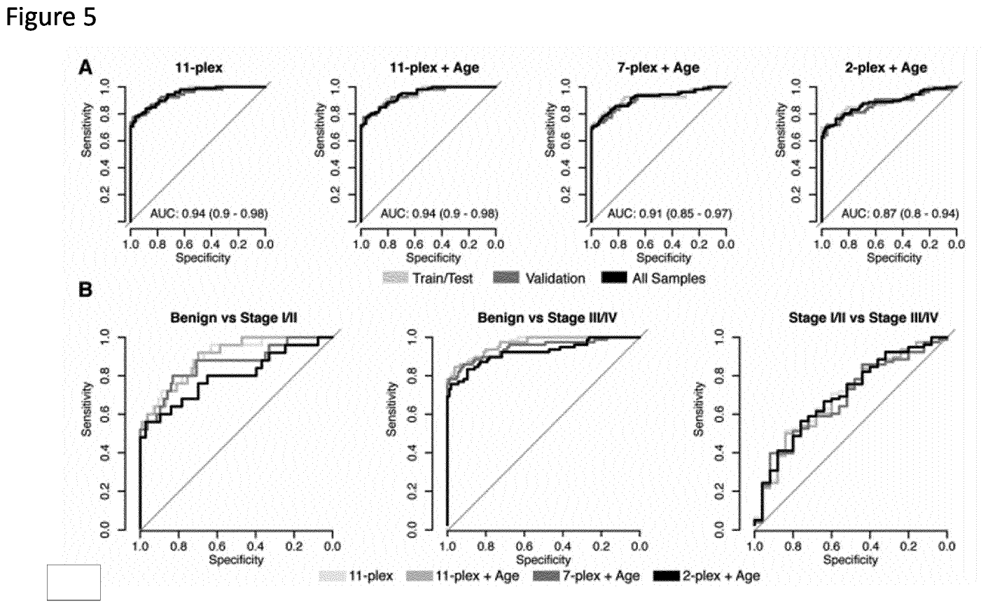

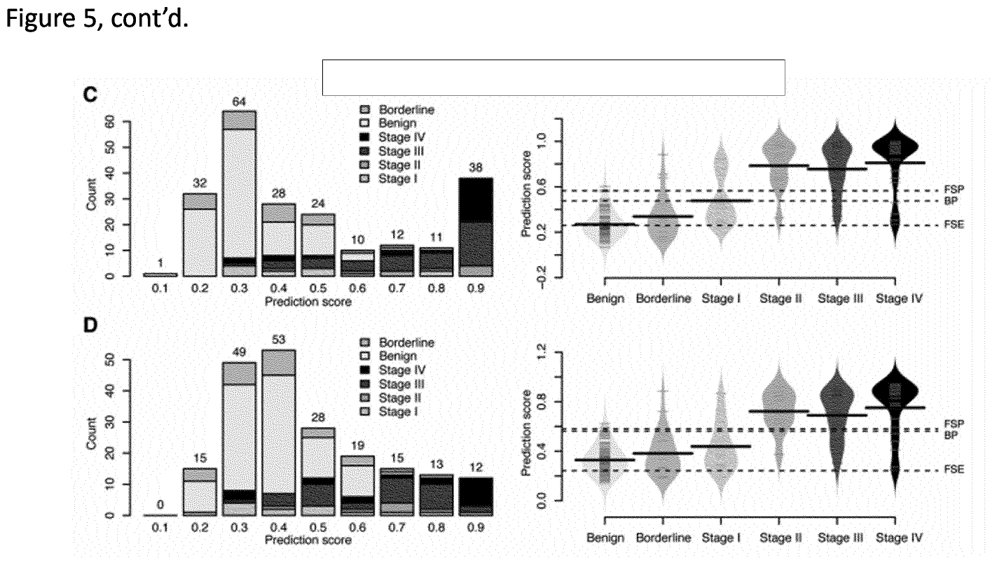

FIG. 5 shows the performance of the final model of Example 4 in the 3^{rd} replication cohort. (A) ROC-curves for the test/training (light grey), validation (dark grey) and final model (black) for each of the 4 models. The AUC is taken from the performance in the validation partition. All models were trained on benign vs malign (stages I-IV) samples. (B) ROC-curves for the 4 final models when evaluated on subsets of stages. (C) Distribution of outcomes in ranges of prediction scores (left) for the ‘11-plex+Age’ model and distribution of prediction scores for each outcome (right). In the right panel, the three cut-offs for ‘best-point (BP)’, ‘focus on sensitivity (FSE, sensitivity 0.98)’ and ‘focus on specificity (FSP, specificity 0.98)’ are illustrated by horizontal dashed lines. The solid black lines indicate mean prediction score in each outcome group. (D) As (C) but for the ‘2-plex+Age’ model.

### EXAMPLES

### Example 1

### Identification of Biomarkers for Ovarian Cancer

### Materials and Methods

Samples Plasma samples of women with ovarian tumours, including benign, either came from the UCAN collection at Uppsala Biobank, Uppsala University, Sweden or the Gynaecology tumour biobank at Sahlgrenska University Hospital, Goteborg, Sweden. All tumours were examined by pathologist specialized in gynaecologic cancers for histology, grade and stage according to International Federation of Gynaecology and Obstetrics (FIGO) standards. All plasma samples were frozen and stored at −70° C. The study was approved by the Regional Ethics Committee in Uppsala (Dnr: 2016/145) and Gothenburg (Dnr: 201-15).

The discovery cohort consisted of 90 patients diagnosed with benign tumours and 79 patients with ovarian cancer stages I-IV. The samples were collected by a trained nurse at time of treatment with sedated patients that have had been fasting as pre-operative preparations declare. The first replication cohort consisted of 71 patients diagnosed with benign tumours and 100 patients with ovarian cancer stages I-IV. The second replication cohort consisted of 77 patients with ovarian cancer stages I-IV. The second replication samples were collected at time of diagnosis by a trained physician in awake patients.

A summary of the cohort statistics is provided in Table 3.

TABLE 3
Cohort statistics.

| | | | No of | Age, | |
|---|---|---|---|---|---|
| Cohort | Origin¹ | Types | Women | mean (SD) | CA-125² |
| Discovery | Gbg | Benign | 90 | 60.0 (16.8) | 16.8 (9.9) |
| | | Tumours | | | |
| | | Stage I-II | 42 | 60.7 (12.4) | 67.6 (72.0) |
| | | Stage III-IV | 37 | 63.8 (14.1) | 327.4 (284.5) |
| 1^{st} | Gbg | Benign | 71 | 60.2 (14.5) | NA |
| Replication | | Tumours | | | |
| | | Stage I-II | 44 | 62.4 (13.7) | NA |
| | | Stage III-IV | 56 | 61.6 (11.3) | NA |
| 2^{nd} | UCAN | Stage I-II | 13 | 55.9 (15.0) | NA |
| Replication | | | | | |
| | | Stage III-IV | 64 | 59.4 (12.0) | NA |

¹UCAN: collection at Uppsala Biobank, Uppsala University, Sweden. Gbg: Gynaecology tumour biobank at Sahlgrenska University Hospital, Göteborg, Sweden
²Measured at clinic [unit], median (median absolute deviation). NA indicates ‘not available’.

### Protein Measurements

The levels of 981 unique proteins were determined by PEA in samples from subjects using multiplexed detection arrays developed by Olink Proteomics. The samples were randomized across chips and normalized for any plate effects using the build in inter-plate controls as by manufacturers recommendations. The PEA gives abundance levels in NPX (Normalized Protein eXpression) that is on log 2-scale. Each assay has a run-time determined lower limit of detection (LOD) defined as three standard deviation above noise level. Here, all assay values below LOD were replaced with the defined LOD-value. Assay characteristics including detection limits calculations, assay performance and validations are available from the manufacturer (www.olink.com).

### Model Generation

First, the discovery set was randomly split into a training set and a test set with 50% of the samples in each, and a linear regression model was generated with the R-package ‘glmnet’ with ‘alpha’=0.9 and optimized using 10-fold cross-validation in the training-set as implemented by the ‘cv.glmnet’-function. This was repeated 50 times with new train/test sets and a core consisting of the proteins present in at least 70% of the generated models was selected. In order to find mutually exclusive cores, the core-generating process was repeated in a recursive manor, excluding one protein at a time from the previous core from the available protein pool. For each newly generated core, the process was then repeated unless the core contained more than a specific number of proteins or had a sensitivity or specificity below a specified cut-off. For each new search, all previously excluded proteins were made unavailable to the current selection. The search was cancelled if more than 20 proteins had been excluded. The core-discovery process is outlined in FIGS. 1A and 1B. For each core, proteins were added creating a final model in a stepwise forward selection. First, the variance in the decision explained by the core was removed by keeping the residuals from a linear model generated with the protein values in the core as input and the decision as output. Then, the variance explained by any other available protein in the adjusted outcome was calculated and the protein explaining the most remaining variance in the decision was added to the model and the contribution of that protein to the explained variance in the decision was adjusted for. This was repeated until the best candidate protein did not explain more than 1% of remaining variance or the total number of proteins in the model exceeded 20 proteins (FIG. 10).

### Results

### Characterization of Plasma Proteins in the Discovery and Replication Cohorts

A total of 552 proteins were characterized in the discovery and replication cohorts using the proximity extension assays (PEA) with the Olink Proseek Cardiometabolic, Cell Regulation, Development, Immune Response, Metabolism and Organ Damage panels (Methods). These measurements complemented a previous study (Enroth et al. Clinical Proteomics 2018, 15: 38) with 460 characterized proteins in the discovery cohort bringing the total number of unique proteins included in the analysis to 981. Forty-two of the 460 proteins have also been quantified in the replication cohorts using the proximity extension assay in two custom 21-plex panels (Enroth et al. ibid). Following quality controls and normalization (Methods), a common set of 593 proteins (42 proteins from the previous 5 panels and 551 from the additional 6 panels) characterized in all samples were used.

### 484 Distinct Predictive Models for Ovarian Cancer

Models were generated using only the discovery data, according to our two-stage strategy. First, mutually exclusive protein cores, consisting of a smaller number of proteins, were selected by repeatedly splitting the data into training and test sets and retaining proteins that were present in at least 70% of the models (Material and Methods, FIG. 1A, B). Additional biomarkers where subsequently added to each core using a stepwise forward selection approach (Material and Methods, FIG. 10). The addition of proteins where terminated when the total model size was 20 proteins, or the next protein to be added did not substantially increase the performance of the model (Material and Methods). Using this strategy, we generated models to distinguish benign tumours from ovarian cancer stages I-II, III-IV and I-IV. For benign tumours versus stages I-II, the core had to be 2-6 proteins in length and have a sensitivity of at least 0.8, or a sensitivity and specificity above 0.6. For stages III-IV, the allowed core size was 2-5, and had to have a sensitivity above 0.8 or a sensitivity and specificity above 0.7. Finally, for stages I-IV the allowed core size was 2-6 proteins, and the models were required to have a sensitivity above 0.8 or a sensitivity and specificity above 0.7. These different parameter settings were used to identify models that can have either high sensitivity or high specificity or both, depending on the final application and to account for the fact that it is much more difficult to separate stages I and II from benign tumours compared to stages III and IV.

The analysis resulted in 484 unique, mutually exclusive, models. MUC-16, which is the clinically most useful single biomarker today, was the most common protein across cores in the 49 shown in Table 5. Our search strategy specifically excludes sets of protein, and 448 of the detected cores did not contain MUC-16. In general, when MUC-16 was not included, the models contained a higher number of proteins (9 to 20) than when it was included (8 to 17). Overall, 371 proteins were included in at least one model. The performance of each of the 49 models and the effect of the addition of each biomarker within each model on the explained variance are shown in FIG. 2A-C.

### Replication of Model Performance

The performance of four models created from the discovery data was then evaluated in two replication cohorts (Models 40, 18, 34 and 48 shown below in Table 6) for stage I-II, stage III-IV, and stage I-IV ovarian cancer. The explained variance for these models is shown in FIG. 2 and Table 4 below, and the maximum explained variance for these models was 83.28%, 82.73%, 82.12% and 82.05%, respectively. Discovery−n=169: 90 benign; 79 ovarian cancer. Replication 1−n=171:71 benign, 100 ovarian cancer. Replication 2−n=148: 71 benign; 77 ovarian cancer. As the second replication cohort lacked patients with benign tumours, the benign tumours from the first replication cohort was used in both replication cohorts. Due to the relativeness of the NPX-scale and that the data in the discovery and replication sets were generated in different laboratory analysis runs, including parts of the data that was generated using a custom-panel, the replication cohorts were split into a test and training set (50-50) and model coefficients were re-determined with the R-package ‘glmnet’. The performance of the models was then estimated in the training set. This was repeated 50 times for each model. The ‘best point’ sensitivity and specificity (defined as the closest (Euclidean distance) point to perfect classification) and AUCs were calculated from the ROC curve. The performance ranges of the models are listed in Table 5.

TABLE 4
marginal increase of explained variance upon addition of biomarkers

| Model | | Biomarkers | | | | | | | | | | | |
|---|---|---|---|---|---|---|---|---|---|---|---|---|---|
| 40 | | SEZ6L2 | IL10 | MUC-16 | CLEC4D | CPE | MSMB | KLK10 | IRF9 | PROK1 | TACSTD2 | CLEC6A | Max |
| | Sum | | 39.76 | 68.8 | 72.79 | 75.49 | 77.01 | 78.47 | 79.76 | 80.71 | 82.36 | 83.28 | 83.28 |
| | ex- | | | | | | | | | | | | |
| | plained | | | | | | | | | | | | |
| | vari- | | | | | | | | | | | | |
| | ance | | | | | | | | | | | | |
| Model | | Biomarkers | | | | | | | | | | | |
| 34 | | SEZ6L2 | PTK7 | MUC-16 | ICOSLG | CLEC6A | MSMB | TACSTD2 | SKAP1 | KLK10 | | | |
| | Sum | | 41.55 | 69.34 | 73 | 75.9 | 77.78 | 80.19 | 81.84 | 82.73 | | | 82.73 |
| | ex- | | | | | | | | | | | | |
| | plained | | | | | | | | | | | | |
| | vari- | | | | | | | | | | | | |
| | ance | | | | | | | | | | | | |
| Model | | Biomarkers | | | | | | | | | | | |
| 18 | | KRT19 | KLK10 | MUC-16 | CLEC4D | ICOSLG | IRF9 | MSMB | TACSTD2 | | | | |
| | Sum | | 42.96 | 71.09 | 75.41 | 78.21 | 79.7 | 80.86 | 82.12 | | | | 82.12 |
| | ex- | | | | | | | | | | | | |
| | plained | | | | | | | | | | | | |
| | vari- | | | | | | | | | | | | |
| | ance | | | | | | | | | | | | |
| Model | | Biomarkers | | | | | | | | | | | |
| 48 | | IL10 | KLK10 | MUC-16 | CPE | IRF9 | CLEC6A | TACSTD2 | PROK1 | | | | |
| | Sum | | 28.53 | 69.49 | 74.39 | 76.96 | 78.48 | 80.58 | 82.05 | | | | 82.05 |
| | ex- | | | | | | | | | | | | |
| | plained | | | | | | | | | | | | |
| | vari- | | | | | | | | | | | | |
| | ance | | | | | | | | | | | | |

TABLE 5
Model performance

| Stage | Model | Cohort | AUC | Sensitivity | Specificity |
|---|---|---|---|---|---|
| I-II | 40 | Discovery | 85% | 83% | 84% |
| | | Replication 1 | 67% | 69% | 70% |
| | | Replication 2 | 81% | NA | NA |
| | 34 | Discovery | 86% | 84% | 86% |
| | | Replication 1 | 66% | 70% | 74% |
| | | Replication 2 | 83% | NA | NA |
| | 18 | Discovery | 90% | 87% | 87% |
| | | Replication 1 | 70% | 67% | 75% |
| | | Replication 2 | 85% | 91% | 82% |
| | 48 | Discovery | 86% | 84% | 86% |
| | | Replication 1 | 69% | 71% | 71% |
| | | Replication 2 | 73% | 79% | 81% |
| III-IV | 40 | Discovery | 96% | 97% | 99% |
| | | Replication 1 | 87% | 87% | 91% |
| | | Replication 2 | 82% | 78% | 85% |
| | 34 | Discovery | 96% | 98% | 99% |
| | | Replication 1 | 87% | 88% | 90% |
| | | Replication 2 | 81% | 80% | 84% |
| | 18 | Discovery | 95% | 98% | 98% |
| | | Replication 1 | 89% | 88% | 92% |
| | | Replication 2 | 77% | 74% | 82% |
| | 48 | Discovery | 96% | 99% | 99% |
| | | Replication 1 | 88% | 87% | 92% |
| | | Replication 2 | 81% | 79% | 84% |
| I-IV | 40 | Discovery | 89% | 87% | 90% |
| | | Replication 1 | 78% | 74% | 84% |
| | | Replication 2 | 77% | 79% | 75% |
| | 34 | Discovery | 91% | 89% | 93% |
| | | Replication 1 | 78% | 77% | 80% |
| | | Replication 2 | 82% | 82% | 81% |
| | 18 | Discovery | 92% | 89% | 91% |
| | | Replication 1 | 82% | 76% | 85% |
| | | Replication 2 | 74% | 73% | 77% |
| | 48 | Discovery | 90% | 87% | 91% |
| | | Replication 1 | 78% | 75% | 84% |
| | | Replication 2 | 78% | 77% | 78% |

### Discussion

### Strategy for Identification of Protein Cores and Additions

The current study was designed to identify mutually exclusive predictive biomarker signatures containing up to 20 proteins differentiating benign conditions from ovarian cancers at different stages. This was done starting from a very large number of plasma proteins. These proteins were not selected based on prior association with ovarian cancer, but because of their availability in high-throughput multiplexed proteomics assays. The prediction models were developed using a discovery cohort, and the performance of the models was then evaluated using two independent replication cohorts. In addition to the hundreds of models obtained using our computerized strategy, we developed one model taking into account protein-specific criteria such as abundance range and sensitivity to haemolysis. Finding combinations of predictive, robust, biomarkers is computationally intensive, and with several hundreds of proteins, exhaustive searches of combinations of up to 20 proteins is not feasible. To this end, we developed a strategy for identification of highly predictive unique signatures using hierarchical exclusion of individual proteins. By design, this lead to the discovery of many signatures that did not contain mucin-16. Overall, the signatures without mucin-16 contained a higher number of proteins than signatures with mucin-16, but there were no clear patterns were either group outperformed the other.

We subsequently evaluated our models in two replication cohorts and found the performance to similar, but somewhat lower than in the discovery set. This either implies that there are underlying differences between the cohorts, such as pre-analytical conditions, or that the models are over-trained with respect to the samples in the discovery cohort. The performance in the test-proportion of the discovery cohort should therefore be considered less certain than the results obtained in the replication cohorts. In our study, the benign tumours and the cancer samples from the 2^{nd} replication cohort differ in pre-analytical context, which could explain part of the lower performance as compared to using the 1^{st} replication cohort. This highlights the importance of understanding the context in which a biomarker test is to be used as compared to the setting used for development of the model.

In summary, we have developed a strategy for identification of protein cores that resulted in mutually exclusive combinations of protein signatures that can separate between benign tumours and ovarian cancers. We also show that broad searches for novel combinations of protein biomarkers that on their own are not necessarily good predictors is a powerful approach for finding relevant biomarkers for disease.

### Example 2

The models shown in Table 6 represent particular combinations of biomarkers according to the present invention. The models were generated following the procedures described in Example 1. Biomarkers for various embodiments of the present invention may therefore be selected from those set forth in the models in Table 6, by selecting any two or more biomarkers (e.g. two, three, four, five, six, seven, eight, nine, ten, eleven or twelve biomarkers) in a model. In particular, the first and second biomarkers in a model may be selected. Optionally, additional biomarkers may also be selected from any one or more of the remaining biomarkers in a model, i.e. from the third biomarker onwards, e.g. by including one or more of the third to the fourth, fifth or sixth biomarkers, or for certain of the models, to the seventh, eighth, ninth, tenth, eleventh or twelfth biomarkers in a model. In particular embodiments, combinations of biomarkers may be selected by selecting additional biomarkers in the order in which they are presented in Table 6, i.e. by including the third biomarker with the first and second biomarkers, and sequentially including the fourth, fifth, sixth biomarkers (and so on) to provide more particular combinations of biomarkers according to the invention. Thus, in certain embodiments, the combination of biomarkers may be provided by selecting the first and second biomarkers, the first to third biomarkers, the first to fourth biomarkers, the first to fifth biomarkers, the first to sixth biomarkers, the first to seventh biomarkers, the first to eighth biomarkers, the first to ninth biomarkers, the first to tenth biomarkers, the first to eleventh biomarkers or the first to twelfth biomarkers as set forth in any of the models shown in Table 6.

TABLE 6

| exemplary panels of biomarkers | | | | | | | |
|---|---|---|---|---|---|---|---|
| Model 1 | TACSTD2 | MUC-16 | ICOSLG | CLEC6A | PROK1 | KLK10 | PROK1 |
| Model 2 | SPINT1 | MUC-16 | ICOSLG | CLEC6A | MSMB | TACSTD2 | |
| Model 3 | KLK10 | MUC-16 | CPE | CLEC6A | TACSTD2 | PROK1 | PROK1 |
| Model 4 | PTK7 | MUC-16 | ICOSLG | MSMB | TACSTD2 | CLEC6A | MSMB |
| Model 5 | SEZ6L2 | MUC-16 | ICOSLG | CLEC6A | TACSTD2 | PROK1 | IRF9 |
| Model 6 | KRT19 | MUC-16 | CLEC4D | ICOSLG | MSMB | TACSTD2 | KLK10 |
| Model 7 | IL10 | MUC-16 | ICOSLG | TACSTD2 | CLEC6A | PROK1 | IRF9 |
| Model 8 | SPINT1 | KRT19 | MUC-16 | CLEC4D | ICOSLG | MSMB | TACSTD2 |
| Model 9 | SPINT1 | KRT19 | CD83 | CD99L2 | MSMB | TANK | PROK1 |
| Model 10 | TACSTD2 | SPINT1 | MUC-16 | CLEC6A | ICOSLG | MSMB | CLMP |
| Model 11 | TACSTD2 | SPINT1 | SPINK5 | SKAP1 | LAMP3 | IL10 | TACSTD2 |
| Model 12 | SPINT1 | PTK7 | SPINK5 | MUC-16 | CLEC6A | ICOSLG | IL10 |
| Model 13 | SPINT1 | PTK7 | SPINK5 | SKAP1 | TACSTD2 | LAMP3 | TACSTD2 |
| Model 14 | SEZ6L2 | SPINT1 | SPINK5 | MUC-16 | CLEC6A | ICOSLG | IL10 |
| Model 15 | SEZ6L2 | SPINT1 | SPINK5 | SKAP1 | TACSTD2 | LAMP3 | ICOSLG |
| Model 16 | SPINT1 | IL10 | SPINK5 | MUC-16 | CLEC6A | TACSTD2 | TANK |
| Model 17 | SPINT1 | IL10 | SPINK5 | SIT1 | TACSTD2 | CLMP | TACSTD2 |
| Model 18 | KRT19 | PTK7 | MUC-16 | CLEC4D | ICOSLG | MSMB | SKAP1 |
| Model 19 | KRT19 | PTK7 | CD83 | MSMB | TANK | TACSTD2 | IRF9 |
| Model 20 | TACSTD2 | KRT19 | MUC-16 | CLEC4D | ICOSLG | MSMB | CLMP |
| Model 21 | TACSTD2 | KRT19 | CD83 | SKAP1 | PTK7 | MSMB | IRF9 |
| Model 22 | SEZ6L2 | KRT19 | MUC-16 | CLEC4D | ICOSLG | MSMB | ICOSLG |
| Model 23 | SEZ6L2 | KRT19 | CLEC4D | FR-alpha | CLMP | IRF9 | PROK1 |
| Model 24 | TACSTD2 | PTK7 | MUC-16 | CLEC6A | ICOSLG | MSMB | TANK |
| Model 25 | TACSTD2 | PTK7 | SKAP1 | CLMP | IL10 | LAMP3 | PROK1 |
| Model 26 | SPINT1 | KLK10 | MUC-16 | CPE | CLEC6A | TACSTD2 | IL10 |
| Model 27 | SPINT1 | KLK10 | SPINK5 | TACSTD2 | SKAP1 | LAMP3 | MSMB |
| Model 28 | KRT19 | KLK10 | MUC-16 | CLEC4D | ICOSLG | IRF9 | SKAP1 |
| Model 29 | KRT19 | KLK10 | CD83 | TANK | WFDC2 | ICOSLG | MSMB |
| Model 30 | TACSTD2 | SEZ6L2 | MUC-16 | ICOSLG | CLEC6A | PROK1 | IL10 |
| Model 31 | TACSTD2 | SEZ6L2 | SKAP1 | SPINK5 | KLK10 | LAMP3 | MSMB |
| Model 32 | IL10 | PTK7 | MUC-16 | ICOSLG | CLEC6A | TACSTD2 | TANK |
| Model 33 | IL10 | PTK7 | CLMP | SIT1 | TACSTD2 | FR-alpha | TACSTD2 |
| Model 34 | SEZ6L2 | PTK7 | MUC-16 | ICOSLG | CLEC6A | MSMB | IL10 |
| Model 35 | SEZ6L2 | PTK7 | SKAP1 | TACSTD2 | CLMP | LAMP3 | TACSTD2 |
| Model 36 | KLK10 | PTK7 | MUC-16 | ICOSLG | SIT1 | MSMB | TANK |
| Model 37 | KLK10 | PTK7 | CLMP | SIT1 | IL10 | TACSTD2 | PROK1 |
| Model 38 | SEZ6L2 | KLK10 | MUC-16 | CPE | CLEC6A | TACSTD2 | IL10 |
| Model 39 | SEZ6L2 | KLK10 | SKAP1 | SPINK5 | TACSTD2 | LAMP3 | KLK10 |
| Model 40 | SEZ6L2 | IL10 | MUC-16 | CLEC4D | CPE | MSMB | KLK10 |
| Model 41 | SEZ6L2 | IL10 | SKAP1 | TACSTD2 | CD83 | CLMP | KLK10 |
| Model 42 | TACSTD2 | IL10 | MUC-16 | ICOSLG | CLEC6A | PROK1 | TANK |
| Model 43 | TACSTD2 | IL10 | SKAP1 | PTK7 | CLMP | LAMP3 | TANK |
| Model 44 | TACSTD2 | KLK10 | MUC-16 | ICOSLG | CLEC6A | PROK1 | IL10 |
| Model 45 | TACSTD2 | KLK10 | SKAP1 | PTK7 | CLMP | TANK | TACSTD2 |
| Model 46 | KRT19 | IL10 | MUC-16 | CLEC4D | ICOSLG | MSMB | CLMP |
| Model 47 | KRT19 | IL10 | CD83 | FR-alpha | IRF9 | ICOSLG | TACSTD2 |
| Model 48 | IL10 | KLK10 | MUC-16 | CPE | IRF9 | CLEC6A | CLMP |
| Model 49 | IL10 | KLK10 | TACSTD2 | SKAP1 | CD83 | PTK7 | |

| | | | | | |
|---|---|---|---|---|---|
| Model 1 | TANK | MSMB | | | |
| Model 2 | | | | | |
| Model 3 | | | | | |
| Model 4 | TANK | | | | |
| Model 5 | KLK10 | | | | |
| Model 6 | | | | | |
| Model 7 | TANK | | | | |
| Model 8 | TACSTD2 | CLEC6A | | | |
| Model 9 | SKAP1 | IL10 | | | |
| Model 10 | | | | | |
| Model 11 | TANK | | | | |
| Model 12 | MSMB | PROK1 | | | |
| Model 13 | CLMP | TANK | | | |
| Model 14 | PROK1 | MSMB | | | |
| Model 15 | CLMP | TANK | | | |
| Model 16 | PROK1 | TANK | | | |
| Model 17 | PROK1 | PTK7 | | | |
| Model 18 | IRF9 | | | | |
| Model 19 | IL10 | CLMP | KLK10 | PROK1 | |
| Model 20 | | | | | |
| Model 21 | TANK | IL10 | KLK10 | PROK1 | |
| Model 22 | TACSTD2 | | | | |
| Model 23 | LAMP3 | | | | |
| Model 24 | TANK | KLK10 | | | |
| Model 25 | SPINK5 | KLK10 | | | |
| Model 26 | | | | | |
| Model 27 | TANK | CLMP | | | |
| Model 28 | TACSTD2 | | | | |
| Model 29 | TACSTD2 | MSMB | PTK7 | CLEC6A | |
| Model 30 | KLK10 | TANK | | | |
| Model 31 | TANK | CLMP | PROK1 | WFDC2 | CLEC6A |
| Model 32 | PROK1 | TANK | | | |
| Model 33 | PROK1 | KLK10 | | | |
| Model 34 | SKAP1 | KLK10 | | | |
| Model 35 | TANK | SPINK5 | KLK10 | WFDC2 | PROK1 |
| Model 36 | PROK1 | CLEC6A | | | |
| Model 37 | PROK1 | FR-alpha | | | |
| Model 38 | | | | | |
| Model 39 | TANK | CLMP | PROK1 | WFDC2 | CLEC6A |
| Model 40 | IRF9 | PROK1 | TACSTD2 | CLEC6A | |
| Model 41 | PROK1 | TANK | WFDC2 | CLEC6A | |
| Model 42 | TANK | | | | |
| Model 43 | SPINK5 | KLK10 | | | |
| Model 44 | MSMB | | | | |
| Model 45 | CD83 | PROK1 | WFDC2 | | |
| Model 46 | IRF9 | | | | |
| Model 47 | | | | | |
| Model 48 | PROK1 | | | | |
| Model 49 | TANK | PROK1 | WFDC2 | | |

### Example 3

### Model Validation

Several factors in addition to the ability to separate cases and controls may influence the choice of the proteins included in a multiplex test, such comparison with established tests, measurable abundance level range, and sensitivity of proteins to haemolysis of red blood cells causing leakage of proteins into the plasma. Taking these limitations into account, we restricted our search to proteins present in models with the highest performance in the discovery cohort. This list of possible additions was filtered by removing proteins sensitive to exposure to hemolysate and proteins that occur in much higher concentrations in human plasma than those in the selected core, and therefore would need to be diluted before assayed with PEA. We then performed model selection as before based solely on the discovery data (benign tumours versus ovarian cancer stages III-IV) and identified a model consisted of 8 proteins. We finally added three proteins (WFDC2, KRT19 and FR-alpha). The protein panel included MUCIN-16, SPINT1, TACSTD2, CLEC6A, ICOSLG, MSMB, PROK1, CDH3, WFDC2, KRT19 and FR-alpha.

The distribution of abundance levels of an initial group of biomarkers in samples from subjects with benign ovarian tumours and subjects with ovarian cancer stages III-IV is shown in FIG. 3A. These biomarkers were found to have deviating levels between the two groups. This is further illustrated by a principle component analysis (PCA) based on each of the biomarkers (FIG. 3B).

The levels of additional biomarkers were found not to differ significantly in abundance between cases and controls when examined separately, but contribute to the separation when examined in combination with the initially identified biomarkers.

The performance of the panel was then evaluated in the two replication cohorts. Models were assembled by sequentially adding biomarkers in the order (i)-(xi). Receiver Operating Characteristic (ROC) curves for benign tumours versus ovarian cancer stages I-II, III-IV and I-IV in the discovery cohort for each biomarker are shown in FIG. 3C. Positive predicted value (PPV) and negative predicted value (NPV) were calculated for each biomarker (FIG. 3D).

In the 1^{st} replication cohort the AUC=0.90, PPV=0.94, sensitivity=0.91 and specificity=0.95 to distinguish benign tumours from ovarian cancer stage III-IV.

A corresponding analysis was performed using the above model lacking CDH3. Results of this analysis are shown in FIG. 4. AUC, sensitivity and specificity data for the discovery cohort and replication cohorts 1 and 2 are shown in Table 7, for the addition of each biomarker in order (starting with TACSTD2, SPINT1, MUC-16, and adding in turn, CLEC6A, ICOSLG, MSMB, PROK1, KRT19, WFDC2 and FR-alpha. Together, this shows that CDH3 can be removed from this model without adversely affecting performance.

TABLE 7
results of model lacking CDH3

| | Dataset | | | | | | | | | |
|---|---|---|---|---|---|---|---|---|---|---|
| Cohort | Stage | Parameter | 1-3 | 4 | 5 | 6 | 7 | 8 | 9 | 10 |
| Explained | — | — | 72.74% | 75.72% | 78.72% | 80.72% | 81.94% | 82.77% | 82.78% | 82.79% |
| variance | | | | | | | | | | |
| Discovery | Benign | AUC | 0.86 | 0.83 | 0.84 | 0.84 | 0.85 | 0.85 | 0.85 | 0.87 |
| | vs I-II | | | | | | | | | |
| | | Mean | 0.85 | 0.82 | 0.81 | 0.81 | 0.83 | 0.83 | 0.83 | 0.84 |
| | | Sensitivity | | | | | | | | |
| | | Stdev | 0.08 | 0.1 | 0.09 | 0.08 | 0.1 | 0.08 | 0.08 | 0.08 |
| | | Sensitivity | | | | | | | | |
| | | Mean | 0.85 | 0.84 | 0.85 | 0.86 | 0.86 | 0.86 | 0.86 | 0.86 |
| | | Specificity | | | | | | | | |
| | | Stdev | 0.08 | 0.1 | 0.11 | 0.1 | 0.1 | 0.09 | 0.09 | 0.08 |
| | | Specificity | | | | | | | | |
| | Benign | AUC | 0.95 | 0.95 | 0.96 | 0.96 | 0.95 | 0.95 | 0.95 | 0.96 |
| | vs III-IV | | | | | | | | | |
| | | Mean | 0.94 | 0.98 | 0.96 | 0.97 | 0.97 | 0.97 | 0.98 | 0.99 |
| | | Sensitivity | | | | | | | | |
| | | Stdev | 0.07 | 0.04 | 0.06 | 0.05 | 0.05 | 0.05 | 0.05 | 0.03 |
| | | Sensitivity | | | | | | | | |
| | | Mean | 0.99 | 0.99 | 0.99 | 0.99 | 0.99 | 0.98 | 0.99 | 0.99 |
| | | Specificity | | | | | | | | |
| | | Stdev | 0.03 | 0.02 | 0.02 | 0.02 | 0.02 | 0.03 | 0.02 | 0.02 |
| | | Specificity | | | | | | | | |
| | Benign | AUC | 0.88 | 0.89 | 0.91 | 0.89 | 0.89 | 0.89 | 0.89 | 0.89 |
| | vs I-IV | | | | | | | | | |
| | | Mean | 0.85 | 0.86 | 0.87 | 0.87 | 0.86 | 0.86 | 0.85 | 0.86 |
| | | Sensitivity | | | | | | | | |
| | | Stdev | 0.06 | 0.06 | 0.08 | 0.05 | 0.08 | 0.05 | 0.06 | 0.07 |
| | | Sensitivity | | | | | | | | |
| | | Mean | 0.9 | 0.92 | 0.91 | 0.91 | 0.91 | 0.92 | 0.92 | 0.92 |
| | | Specificity | | | | | | | | |
| | | Stdev | 0.07 | 0.05 | 0.06 | 0.06 | 0.06 | 0.06 | 0.06 | 0.06 |
| | | Specificity | | | | | | | | |
| Replication 1 | Benign | AUC | 0.68 | 0.71 | 0.66 | 0.69 | 0.65 | 0.68 | 0.7 | 0.74 |
| | vs I-II | | | | | | | | | |
| | | Mean | 0.72 | 0.75 | 0.72 | 0.69 | 0.7 | 0.68 | 0.75 | 0.76 |
| | | Sensitivity | | | | | | | | |
| | | Stdev | 0.14 | 0.11 | 0.12 | 0.11 | 0.13 | 0.14 | 0.13 | 0.12 |
| | | Sensitivity | | | | | | | | |
| | | Mean | 0.69 | 0.71 | 0.67 | 0.75 | 0.72 | 0.76 | 0.73 | 0.75 |
| | | Specificity | | | | | | | | |
| | | Stdev | 0.1 | 0.11 | 0.11 | 0.1 | 0.13 | 0.14 | 0.11 | 0.1 |
| | | Specificity | | | | | | | | |
| | Benign | AUC | 0.85 | 0.9 | 0.89 | 0.87 | 0.88 | 0.89 | 0.9 | 0.91 |
| | vs III-IV | | | | | | | | | |
| | | Mean | 0.86 | 0.9 | 0.89 | 0.88 | 0.88 | 0.9 | 0.89 | 0.9 |
| | | Sensitivity | | | | | | | | |
| | | Stdev | 0.07 | 0.06 | 0.06 | 0.06 | 0.07 | 0.06 | 0.06 | 0.08 |
| | | Sensitivity | | | | | | | | |
| | | Mean | 0.9 | 0.91 | 0.93 | 0.91 | 0.91 | 0.94 | 0.93 | 0.94 |
| | | Specificity | | | | | | | | |
| | | Stdev | 0.08 | 0.07 | 0.06 | 0.07 | 0.06 | 0.06 | 0.07 | 0.07 |
| | | Specificity | | | | | | | | |
| | Benign | AUC | 0.8 | 0.78 | 0.79 | 0.79 | 0.79 | 0.79 | 0.82 | 0.82 |
| | vs I-IV | | | | | | | | | |
| | | Mean | 0.8 | 0.78 | 0.78 | 0.77 | 0.79 | 0.77 | 0.8 | 0.81 |
| | | Sensitivity | | | | | | | | |
| | | Stdev | 0.06 | 0.07 | 0.08 | 0.08 | 0.06 | 0.08 | 0.07 | 0.07 |
| | | Sensitivity | | | | | | | | |
| | | Mean | 0.82 | 0.79 | 0.79 | 0.84 | 0.82 | 0.84 | 0.84 | 0.84 |
| | | Specificity | | | | | | | | |
| | | Stdev | 0.07 | 0.09 | 0.08 | 0.08 | 0.08 | 0.08 | 0.07 | 0.08 |
| | | Specificity | | | | | | | | |
| Replication 2 | Benign | AUC | 0.76 | 0.83 | 0.79 | 0.78 | 0.73 | 0.77 | 0.75 | 0.73 |
| | vs I-II | | | | | | | | | |
| | | Mean | 0.84 | 0.88 | NA | 0.83 | 0.8 | 0.81 | NA | 0.78 |
| | | Sensitivity | | | | | | | | |
| | | Stdev | 0.17 | 0.18 | NA | 0.16 | 0.18 | 0.18 | NA | 0.21 |
| | | Sensitivity | | | | | | | | |
| | | Mean | 0.8 | 0.84 | NA | 0.81 | 0.78 | 0.82 | NA | 0.78 |
| | | Specificity | | | | | | | | |
| | | Stdev | 0.14 | 0.13 | NA | 0.14 | 0.17 | 0.19 | NA | 0.17 |
| | | Specificity | | | | | | | | |
| | Benign | AUC | 0.84 | 0.85 | 0.83 | 0.81 | 0.84 | 0.83 | 0.83 | 0.86 |
| | vs III-IV | | | | | | | | | |
| | | Mean | 0.82 | 0.8 | 0.81 | 0.78 | 0.82 | 0.82 | 0.8 | 0.83 |
| | | Sensitivity | | | | | | | | |
| | | Stdev | 0.08 | 0.09 | 0.1 | 0.08 | 0.08 | 0.08 | 0.08 | 0.09 |
| | | Sensitivity | | | | | | | | |
| | | Mean | 0.9 | 0.91 | 0.88 | 0.88 | 0.91 | 0.87 | 0.87 | 0.88 |
| | | Specificity | | | | | | | | |
| | | Stdev | 0.08 | 0.07 | 0.07 | 0.07 | 0.06 | 0.08 | 0.09 | 0.08 |
| | | Specificity | | | | | | | | |
| | Benign | AUC | 0.81 | 0.81 | 0.79 | 0.81 | 0.78 | 0.81 | 0.83 | 0.83 |
| | vs I-IV | | | | | | | | | |
| | | Mean | 0.79 | 0.79 | 0.76 | 0.78 | 0.74 | 0.77 | 0.81 | 0.81 |
| | | Sensitivity | | | | | | | | |
| | | Stdev | 0.08 | 0.08 | 0.08 | 0.08 | 0.1 | 0.08 | 0.09 | 0.09 |
| | | Sensitivity | | | | | | | | |
| | | Mean | 0.85 | 0.85 | 0.85 | 0.82 | 0.82 | 0.84 | 0.83 | 0.82 |
| | | Specificity | | | | | | | | |
| | | Stdev | 0.08 | 0.09 | 0.1 | 0.1 | 0.07 | 0.09 | 0.08 | 0.08 |
| | | Specificity | | | | | | | | |

### Example 4

### Further Study of 11-Plex Model of Example 3 and Development of 7-Plex Model Based Thereon

The model of Example 3 based on the 11-plex biomarker panel provided a proof-of concept model which was studied and developed further. In an extension of Examples 1 and 3, the “11-plex” model was implemented as a custom multiplex PEA assay reporting in absolute concentrations and the performance of this model was validated in a third, independent, replication cohort.

### Materials and Methods (Additional to Example 1)

### Samples

The third replication cohort consisted of 106 patients with benign conditions, 28 with borderline diagnosis and 103 with ovarian cancer stages I-IV. All samples from the third replication cohort were collected at time of diagnosis, from awake patients, by a trained nurse.

### Protein Measurements

The proteins from the proof-of-concept model (of Example 3) were quantified using a custom 11-plex assay in the analysis of the third replication cohort. Description of the development process for combining protein assays into custom multiplexed reactions and the technology behind having final readout in absolute concentrations has been published earlier (Assarsson, E. & Lundberg, M. Development and validation of customized PEA biomarker panels with clinical utility. Advancing precision medicine: Current and future proteogenomic strategies for biomarker discovery and development (Science/AAAS, Washington, D.C., 2017), 32-36 (2017). In brief, normal and disease state occurring protein concentration ranges in circulating plasma are accounted for and the dynamic range for each individual assay optimized to take this into account. In addition, standard curves for all individual proteins have to be established by analysis of a wide range of recombinant antigen concentrations. In the final test, triplicate measures of calibrators at 4 known concentrations (blank, low, mid and high concentrations) of each protein were included in each run. These were used for normalization and the normal PEA-readout (NPX) and to estimate absolute concentrations by comparing to established standard curves. Here, each sample in the third replication cohort was run in duplicates or triplicates and a mean value over the replicates was used in the analyses. If all readouts were below or above the limits of detections, no mean-value was calculated but instead replaced with the LOD-values as described above. The data was then transformed to log 2-scale.

### Fixation of the Models in Absolute Concentration

The third replication cohort and was first split into two equal parts, a training set and a validation set, in terms of size and proportion of benign and malign (stages I-IV) samples. A linear regression model was then trained employing fivefold cross-validation using the training part only. The models were trained using the ‘cv.glmnet’ with alpha=0. The performance of the model was then evaluated on the validation set. Difference in performance (AUC) from the training and validation was evaluated by a DeLong-test as implemented in the R-package ‘pRoc’. When no difference in performance was detected between the training and validation sets, a final model was generated fivefold cross-validation as above using all samples with benign or malign (stages I-IV) status. Model coefficients was extracted from the cv-stage at a λ within one standard error of the minimum (‘lambda.1se’ in ‘cv.glmnet’).

### Results

### Validation of Proof-of-Concept Model

In order to validate the performance of the 11-protein proof-of-concept model we then developed a custom PEA-assay that measured the 11 proteins and used this to characterize protein abundance levels in a third replication cohort (Table 8). Here, calibration samples (see Methods for details) were included in the custom assay in order to have the final readout in absolute protein concentrations rather than NPX. Concentration ranges of the custom assay and performance measures are given in Table 9. The third replication cohort was first split into two equal parts, a training set and a validation set, in terms of size and proportion of benign and malignant (stages I-IV) tumors. A linear regression model was then trained, employing fivefold cross-validation using the training part only. In the training-set this model achieved an AUC of 0.94 (%95 CI 0.91-0.98) in separating benign from stages I-IV (malignant), and a similar performance was observed in the validation set (AUC=0.94, %95 CI 0.90-0.98, FIG. 5A). Since the performance in the validation set was highly similar to the training set with no statistical difference (DeLongs's test, p-value=0.89), a final model was generated using fivefold cross-validation with the entire third replication cohort in order to capture as much variation as possible. This model (Table 9) achieved an AUC of 0.95 (%95 CI 0.92-0.97) with a sensitivity of 0.78 at a specificity of 0.96 at the point closest to perfect classification. Next, we trained a model using the 11 proteins and age at diagnosis (Table 9, FIG. 5A). As before, there was no difference in AUC for the training and validation sets (DeLongs's test, p-value=0.96) and using the whole cohort, this model achieved an AUC of 0.94 (%95 CI 0.92-0.97) with a sensitivity of 0.78 at a specificity of 0.95 at the point closest to perfect classification. This was determined at a cut-off of 0.4758. We also recorded cut-offs for focus on sensitivity or specificity over 0.98. With this focus, the model achieved sensitivity and specificity of 0.98/0.58 or 0.73/0.98 at cut-offs of 0.2599 and 0.5647 respectively (Table 8). We also trained a model based on WFDC2, Mucin-16 and age at diagnose for comparison and a model based on age and 7 biomarkers (MUCIN-16, TACSTD2, MSMB, PROK1, WFDC2, KRT19 and FR-alpha) that excluded the proteins with the highest technical variation in our custom-assay. In both these models there was no difference in performance between the training and validation proportions of the data (DeLong's test p-values=0.75 and 0.89 respectively) and again, final models were created based on the entire cohort. Performance measures for all 4 models based on the custom assay are available in Table 10. In general, the models trained on benign vs malignant (stages I-IV) tumors are better at separating late stages (stages III-IV, AUC-range 0.92-0.97) than early (stages I-II, AUC-range 0.80-90) and has poorer performance separating stages I-II from stages III-IV (AUC-range 0.68-0.70, FIG. 5B, Table 10).

Finally, we included also samples from the third replication cohort that had been diagnosed with borderline ovarian cancer and plotted the prediction scores from the 11 proteins plus age model alongside of the benign and malignant samples (FIG. 5C). From FIG. 5C left panel it is clear that only samples with stages III or higher have prediction scores above 0.9 while all benign or borderline samples have a score lower than 0.2. As compared to the use of the WFDC2, MUCIN-16 plus age model (FIG. 5D, left panel), there is a more than 2-fold increase in the number of women that fall in these prediction score categories, i.e. above 0.9 or below 0.2. This is also illustrated by FIG. 5CD, right panels, where the distribution of prediction scores for each diagnose is shown. The cut-offs used for “best point”, high sensitivity or high specificity are also illustrated by horizontal lines. The prediction scores for the borderline samples falls between the benign and stage I samples (FIG. 5C, right panel) and there is not obvious cut-off that for distinguishing these from either the benign or malignant tumor samples.

### Discussion

The performance of the final, proof-of-concept, model is slightly better in the third replication cohort, where the AUC was 0.94 (%95 CI 0.90-0.98) in the validation-proportion, as compared to the performance in the test-proportion of the discovery and first two replication cohorts, where the model had AUCs ranging from 0.82-0.88. This could be due to the wider dynamic range of the custom assay, but indicates that the performance of the model is robust. A second contributing factor could be that cases here are compared to a group diagnosed with benign tumors, representing heterogenous conditions. In the third replication cohort used here, the most common benign tumors were diagnosed as ‘Serous cyst’ (29.2%), ‘Mucinous cyst’ (20.8%), ‘Simple cyst’ (17.0%), ‘Stromal cyst’ (11.3%), ‘Teratoma cyst’ (11.3%), ‘Endometriosis’ (6.6%) and finally, ‘Myoma cyst’ (3.8%). This highlights the importance of understanding the context in which a biomarker test is to be used as compared to the setting used for development of the model.

TABLE 8
Performance of the custom assay in the independent replication cohort

| | Full model¹ | | | | MUCIN-16 and WFDC2 and Age | | | |
|---|---|---|---|---|---|---|---|---|
| | Benign vs | Benign vs | Benign vs | Stage I-II vs | Benign vs | Benign vs | Benign vs | Stage I-II vs |
| | Malign | Stage I-II | Stage III-IV | Stage III-IV | Malign | Stage I-II | Stage III-IV | Stage III-IV |
| AUC | 0.94 | 0.89 | 0.96 | 0.69 | 0.88 | 0.8 | 0.92 | 0.7 |
| | (0.92-0.97) | (0.83-0.96) | (0.94-0.99) | (0.57-0.81) | (0.84-0.93) | (0.68-0.91) | (0.88-0.97) | (0.58-0.81) |
| PPV² | 0.94 | 0.75 | 0.93 | 0.81 | 0.96 | 0.82 | 0.95 | 0.81 |
| NPV² | 0.81 | 0.91 | 0.89 | 0.45 | 0.76 | 0.9 | 0.84 | 0.37 |
| BPcut³ | 0.4758 | 0.4758 | 0.4758 | 0.4758 | 0.5625 | 0.5625 | 0.5625 | 0.5625 |
| BPse | 0.78 | 0.60 | 0.85 | 0.85 | 0.70 | 0.56 | 0.76 | 0.76 |
| | (0.70-0.86) | (0.40-0.80) | (0.76-0.92) | (0.76-0.92) | (0.60-0.78) | (0.36-0.76) | (0.67-0.85) | (0.65-0.86) |
| BPsp | 0.95 | 0.95 | 0.95 | 0.4 | 0.97 | 0.97 | 0.97 | 0.44 |
| | (0.91-0.99) | (0.91-0.99) | (0.91-0.99) | (0.20-0.60) | (0.93-1.00) | (0.93-1.00) | (0.93-1.00) | (0.24-0.64) |
| FSEcut³ | 0.2599 | 0.2599 | 0.2599 | 0.2599 | 0.243 | 0.243 | 0.243 | 0.243 |
| FSEse | 0.98 | 0.96 | 1.00 | 1.00 | 0.98 | 0.96 | 1.00 | 1.00 |
| | (0.95-1.00) | (0.88-1.00) | (1.00-1.00) | (1.00-1.00) | (0.95-1.00) | (0.88-1.00) | (1.00-1.00) | (1.00-1.00) |
| FSEsp | 0.58 | 0.58 | 0.58 | 0.04 | 0.25 | 0.25 | 0.25 | 0.04 |
| | (0.49-0.68) | (0.50-0.68) | (0.49-0.68) | (0.00-0.12) | (0.16-0.33) | (0.17-0.33) | (0.17-0.33) | (0.00-0.12) |
| FSPcut³ | 0.5647 | 0.5647 | 0.5647 | 0.5647 | 0.5805 | 0.5805 | 0.5805 | 0.5805 |
| FSPse | 0.73 | 0.56 | 0.79 | 0.79 | 0.68 | 0.48 | 0.76 | 0.76 |
| | (0.64-0.80) | (0.36-0.76) | (0.71-0.88) | (0.71-0.88) | (0.58-0.76) | (0.28-0.68) | (0.67-0.85) | (0.67-0.85) |
| FSPsp | 0.98 | 0.98 | 0.98 | 0.44 | 0.98 | 0.98 | 0.98 | 0.52 |
| | (0.95-1.00) | (0.95-1.00) | (0.95-1.00) | (0.24-0.64) | (0.95-1.00) | (0.95-1.00) | (0.95-1.00) | (0.32-0.72) |

¹Proof-of-concept model plus age.
²Performances when cut-off is chosen at the best point (BP, closest point on ROC-curve to perfect classification).
³Cut-off thresholds calculated in the Benign vs. Malign models and applied to difference subgroups. The BPcut is taken at the point on the ROC-curve closest to perfect performace. The FSEcut is taken from point with highest specificity when requiring at least 0.98 sensitivity. The FSPcut is taken from point with highest sensitivity when requiring at least 0.98 specificity. All cells: numbers in parentheses represent 95% confidence intervals

TABLE 9

| | Custom Assay Performance Measures | | | | | | |
|---|---|---|---|---|---|---|---|
| | CV | | | | | | |
| | | Infra | Infra | | | | Model |
| | Inter | Avg. | Max | | Assay range | | characteristics |
| variable | (%) | (%) | (%) | unit | LLOQ | ULOQ | unit |
| (Intercept) | | | | | | | dimensionless |
| PROK1 | 5 | 6 | 17 | pg/ml | 15.92029 | 16883.06305 | log2(pg/mL) |
| WFDC2 | 5 | 7 | 20 | pg/ml | 395.26602 | 181210.62850 | log2(pg/mL) |
| KRT19 | 2 | 4 | 7 | U/ml | 74600.71807 | 68900244.47955 | log2(U/m1) |
| FR.alpha | 3 | 6 | 11 | pg/ml | 7.79496 | 163421.40351 | log2(pg/mL) |
| ICOSLG | 4 | 6 | 16 | pg/ml | 62.41692 | 55648.31473 | log2(pg/mL) |
| CDH3 | 3 | 6 | 16 | pg/ml | 278.80868 | 638185.76563 | log2(pg/mL) |
| MSMB | 4 | 5 | 14 | pg/ml | 323.21595 | 177291.40831 | log2(pg/mL) |
| CLEC6A | 5 | 9 | 20 | pg/ml | 2.06011 | 13291.04962 | log2(pg/mL) |
| TACSTD2 | 2 | 5 | 11 | pg/ml | 3.86544 | 33922.31855 | log2(pg/mL) |
| MUC.16 | 5 | 7 | 24 | mU/m1 | 2346.83121 | 2288168.66936 | log2(mU/ml) |
| SPINT1 | 11 | 10 | 28 | pg/ml | 1347.89124 | 193069.95222 | log2(pg/mL) |
| ageAtDiag | | | | | | | years |
| | | Model characteristics | | | | | |
| | | Sample range in training | | | | | |
| | variable | low | high | 11-plex | 11-plex + Age | 7-plex + Age | 2-plex + Age |
| | (Intercept) | | | 0.368297347 | 0.064756779 | −12.30005072 | −9.369273745 |
| | PROK1 | 4.317450965 | 9.623530867 | −0.373965688 | −0.355189031 | −0.367641882 | |
| | WFDC2 | 10.40270848 | 16.99555459 | 0.295831446 | 0.287713864 | 0.302906037 | 0.409074851 |
| | KRT19 | 18.84105774 | 26.03800577 | 0.257954212 | 0.253974658 | 0.278052005 | |
| | FR.alpha | 9.348167319 | 15.81082719 | 0.208467863 | 0.204150142 | 0.201552918 | |
| | ICOSLG | 11.80111643 | 13.45897223 | −1.111253263 | −1.103303467 | | |
| | CDH3 | 14.01276568 | 19.2836169 | −0.119382413 | −0.116724712 | | |
| | MSMB | 8.924573415 | 15.45268003 | −0.091353541 | −0.105847092 | −0.137883386 | |
| | CLEC6A | 1.842180353 | 5.795463844 | −0.205347449 | −0.215965658 | | |
| | TACSTD2 | 6.63744607 | 11.17277258 | 0.175511303 | 0.17754229 | 0.193935338 | |
| | MUC.16 | 11.95268078 | 21.12576197 | 0.145892175 | 0.148708579 | 0.163795221 | 0.218757952 |
| | SPINT1 | 12.61474137 | 17.55876413 | 0.334760093 | 0.338463976 | | |
| | ageAtDiag | 21 | 94 | | 0.006271834 | 0.005683631 | 0.009856458 |

TABLE 10

| | | | | | | | Difference in | | |
|---|---|---|---|---|---|---|---|---|---|
| | Performance Measure | | | | | | performance (AUC) Training | | |
| Model | AUC | cutoff | Sensitivity | Specificity | PPV | NPV | test | statistica | p-value |
| 11-plex | | | | | | | | | |
| Training | 0.94 (0.91-0.98) | | | | | | | | |
| bp | | 0.4174 | 0.81 (0.70-0.91) | 0.92 (0.85-0.98) | 0.91 | 0.83 | | | |
| fse | | 0.2634 | 0.98 (0.94-1.00) | 0.57 (0.43-0.70) | 0.69 | 0.97 | | | |
| fsp | | 0.5818 | 0.74 (0.60-0.85) | 0.98 (0.94-1.00) | 0.97 | 0.79 | | | |
| Validation | 0.94 (0.9-0.98) | | | | | | DeLong's | 0.1349293 | 0.8927984 |
| bp | | 0.4174 | 0.85 (0.75-0.94) | 0.87 (0.77-0.96) | 0.87 | 0.85 | | | |
| fse | | 0.2634 | 0.96 (0.91-1.00) | 0.55 (0.42-0.68) | 0.68 | 0.94 | | | |
| fsp | | 0.5818 | 0.72 (0.58-0.83) | 1 (1.00-1.00) | 1 | 0.78 | | | |
| Combined | 0.95 (0.92-0.97) | | | | | | | | |
| bp | | 0.4729 | 0.78 (0.70-0.86) | 0.96 (0.92-0.99) | 0.95 | 0.82 | | | |
| fse | | 0.2756 | 0.98 (0.95-1.00) | 0.63 (0.54-0.72) | 0.73 | 0.97 | | | |
| fsp | | 0.5408 | 0.74 (0.65-0.81) | 0.98 (0.95-1.00) | 0.97 | 0.79 | | | |
| Benign_vs_StageI/II | 0.9 (0.83-0.96) | | | | | | | | |
| bp | | 0.4729 | 0.60 (0.40-0.80) | 0.96 (0.92-0.99) | 0.79 | 0.91 | | | |
| fse | | 0.2756 | 0.96 (0.88-1.00) | 0.63 (0.54-0.72) | 0.38 | 0.99 | | | |
| fsp | | 0.5408 | 0.56 (0.36-0.76) | 0.98 (0.95-1.00) | 0.88 | 0.9 | | | |
| Benign_vs_StageIII/IV | 0.97 (0.94-0.99) | | | | | | | | |
| bp | | 0.4729 | 0.85 (0.76-0.92) | 0.96 (0.92-0.99) | 0.94 | 0.89 | | | |
| fse | | 0.2756 | 1.00 (1.00-1.00) | 0.63 (0.54-0.72) | 0.67 | 1 | | | |
| fsp | | 0.5408 | 0.81 (0.73-0.90) | 0.98 (0.95-1.00) | 0.97 | 0.87 | | | |
| StageI/II_vs_StageIII/IV | 0.7 (0.58-0.81) | | | | | | | | |
| bp | | 0.4729 | 0.85 (0.76-0.92) | 0.4 (0.20-0.60) | 0.81 | 0.45 | | | |
| fse | | 0.2756 | 1.00 (1.00-1.00) | 0.04 (0.00-0.12) | 0.76 | 1 | | | |
| fsp | | 0.5408 | 0.81 (0.72-0.88) | 0.44 (0.24-0.64) | 0.82 | 0.42 | | | |
| 11-plex + Age | | | | | | | | | |
| Training | 0.94 (0.9-0.98) | | | | | | | | |
| bp | | 0.4206 | 0.81 (0.70-0.91) | 0.92 (0.85-0.98) | 0.91 | 0.83 | | | |
| fse | | 0.2536 | 0.98 (0.94-1.00) | 0.55 (0.42-0.68) | 0.68 | 0.97 | | | |
| fsp | | 0.5902 | 0.74 (0.60-0.85) | 0.98 (0.94-1.00) | 0.97 | 0.79 | | | |
| Validation | 0.94 (0.9-0.98) | | | | | | DeLong's | 0.04909646 | 0.9608891 |
| bp | | 0.4206 | 0.85 (0.75-0.94) | 0.87 (0.77-0.96) | 0.87 | 0.85 | | | |
| fse | | 0.2536 | 0.98 (0.94-1.00) | 0.53 (0.40-0.66) | 0.68 | 0.97 | | | |
| fsp | | 0.5902 | 0.72 (0.58-0.83) | 1 (1.00-1.00) | 1 | 0.78 | | | |
| Combined | 0.94 (0.92-0.97) | | | | | | | | |
| bp | | 0.4758 | 0.78 (0.70-0.86) | 0.95 (0.91-0.99) | 0.94 | 0.81 | | | |
| fse | | 0.2599 | 0.98 (0.95-1.00) | 0.58 (0.49-0.68) | 0.7 | 0.97 | | | |
| fsp | | 0.5647 | 0.73 (0.64-0.80) | 0.98 (0.95-1.00) | 0.97 | 0.78 | | | |
| Benign_vs_StageI/II | 0.89 (0.83-0.96) | | | | | | | | |
| bp | | 0.4758 | 0.60 (0.40-0.80) | 0.95 (0.91-0.99) | 0.75 | 0.91 | | | |
| fse | | 0.2599 | 0.96 (0.88-1.00) | 0.58 (0.50-0.68) | 0.35 | 0.98 | | | |
| fsp | | 0.5647 | 0.56 (0.36-0.76) | 0.98 (0.95-1.00) | 0.88 | 0.9 | | | |
| Benign_vs_StageIII/IV | 0.96 (0.94-0.99) | | | | | | | | |
| bp | | 0.4758 | 0.85 (0.76-0.92) | 0.95 (0.91-0.99) | 0.93 | 0.89 | | | |
| fse | | 0.2599 | 1.00 (1.00-1.00) | 0.58 (0.49-0.68) | 0.64 | 1 | | | |
| fsp | | 0.5647 | 0.79 (0.71-0.88) | 0.98 (0.95-1.00) | 0.97 | 0.87 | | | |
| StageI/II_vs_StageIII/IV | 0.69 (0.57-0.81) | | | | | | | | |
| bp | | 0.4758 | 0.85 (0.76-0.92) | 0.4 (0.20-0.60) | 0.81 | 0.45 | | | |
| fse | | 0.2599 | 1.00 (1.00-1.00) | 0.04 (0.00-0.12) | 0.76 | 1 | | | |
| fsp | | 0.5647 | 0.79 (0.71-0.88) | 0.44 (0.24-0.64) | 0.82 | 0.41 | | | |
| 7-plex + Age | | | | | | | | | |
| Training | 0.92 (0.86-0.97) | | | | | | | | |
| bp | | 0.3731 | 0.85 (0.75-0.94) | 0.87 (0.77-0.94) | 0.87 | 0.85 | | | |
| fse | | 0.2052 | 0.98 (0.94-1.00) | 0.26 (0.15-0.40) | 0.57 | 0.93 | | | |
| fsp | | 0.5691 | 0.74 (0.60-0.85) | 0.98 (0.94-1.00) | 0.97 | 0.79 | | | |
| Validation | 0.91 (0.85-0.97) | | | | | | DeLong's | 0.1374334 | 0.8908199 |
| bp | | 0.3731 | 0.87 (0.77-0.94) | 0.75 (0.64-0.87) | 0.78 | 0.85 | | | |
| fse | | 0.2052 | 0.96 (0.91-1.00) | 0.23 (0.11-0.34) | 0.55 | 0.86 | | | |
| fsp | | 0.5691 | 0.74 (0.60-0.85) | 0.94 (0.87-1.00) | 0.93 | 0.78 | | | |
| Combined | 0.91 (0.87-0.95) | | | | | | | | |
| bp | | 0.4621 | 0.78 (0.70-0.86) | 0.92 (0.86-0.96) | 0.9 | 0.81 | | | |
| fse | | 0.2119 | 0.98 (0.95-1.00) | 0.24 (0.16-0.32) | 0.56 | 0.93 | | | |
| fsp | | 0.5837 | 0.71 (0.62-0.79) | 0.98 (0.95-1.00) | 0.97 | 0.77 | | | |
| Benign_vs_StageI/II | 0.86 (0.77-0.95) | | | | | | | | |
| bp | | 0.4621 | 0.60 (0.40-0.80) | 0.92 (0.86-0.96) | 0.62 | 0.91 | | | |
| fse | | 0.2119 | 1.00 (1.00-1.00) | 0.24 (0.16-0.32) | 0.24 | 1 | | | |
| fsp | | 0.5837 | 0.52 (0.32-0.72) | 0.98 (0.95-1.00) | 0.87 | 0.9 | | | |
| Benign_vs_StageIII/IV | 0.94 (0.91-0.98) | | | | | | | | |
| bp | | 0.4621 | 0.86 (0.77-0.92) | 0.92 (0.86-0.96) | 0.88 | 0.9 | | | |
| fse | | 0.2119 | 0.99 (0.96-1.00) | 0.24 (0.15-0.32) | 0.49 | 0.96 | | | |
| fsp | | 0.5837 | 0.78 (0.69-0.87) | 0.98 (0.95-1.00) | 0.97 | 0.86 | | | |
| StageI/II_vs_StageIII/IV | 0.68 (0.56-0.8) | | | | | | | | |
| bp | | 0.4621 | 0.86 (0.77-0.94) | 0.4 (0.20-0.60) | 0.82 | 0.48 | | | |
| fse | | 0.2119 | 0.99 (0.96-1.00) | 0 (0.00-0.00) | 0.75 | 0 | | | |
| fsp | | 0.5837 | 0.78 (0.69-0.87) | 0.48 (0.28-0.68) | 0.82 | 0.41 | | | |
| 2-plex + Age | | | | | | | | | |
| Training | 0.89 (0.83-0.95) | | | | | | | | |
| bp | | 0.607 | 0.70 (0.57-0.81) | 1 (1.00-1.00) | 1 | 0.77 | | | |
| fse | | 0.2066 | 0.98 (0.94-1.00) | 0.25 (0.13-0.36) | 0.57 | 0.93 | | | |
| fsp | | 0.5982 | 0.70 (0.57-0.81) | 0.98 (0.94-1.00) | 0.97 | 0.76 | | | |
| Validation | 0.87 (0.8-0.94) | | | | | | DeLong's | 0.3221856 | 0.7476346 |
| bp | | 0.607 | 0.70 (0.57-0.81) | 0.96 (0.91-1.00) | 0.95 | 0.76 | | | |
| fse | | 0.2066 | 0.98 (0.94-1.00) | 0.19 (0.09-0.30) | 0.55 | 0.91 | | | |
| fsp | | 0.5982 | 0.72 (0.58-0.83) | 0.96 (0.91-1.00) | 0.95 | 0.77 | | | |
| Combined | 0.88 (0.84-0.93) | | | | | | | | |
| bp | | 0.5625 | 0.70 (0.60-0.78) | 0.97 (0.93-1.00) | 0.96 | 0.76 | | | |
| fse | | 0.243 | 0.98 (0.95-1.00) | 0.25 (0.16-0.33) | 0.57 | 0.93 | | | |
| fsp | | 0.5805 | 0.68 (0.58-0.76) | 0.98 (0.95-1.00) | 0.97 | 0.75 | | | |
| Benign_vs_StageI/II | 0.8 (0.68-0.91) | | | | | | | | |
| bp | | 0.5625 | 0.56 (0.36-0.76) | 0.97 (0.93-1.00) | 0.82 | 0.9 | | | |
| fse | | 0.243 | 0.96 (0.88-1.00) | 0.25 (0.17-0.33) | 0.23 | 0.96 | | | |
| fsp | | 0.5805 | 0.48 (0.28-0.68) | 0.98 (0.95-1.00) | 0.86 | 0.89 | | | |
| Benign_vs_StageIII/IV | 0.92 (0.88-0.97) | | | | | | | | |
| bp | | 0.5625 | 0.76 (0.67-0.85) | 0.97 (0.93-1.00) | 0.95 | 0.84 | | | |
| fse | | 0.243 | 1.00 (1.00-1.00) | 0.25 (0.17-0.33) | 0.49 | 1 | | | |
| fsp | | 0.5805 | 0.76 (0.67-0.85) | 0.98 (0.95-1.00) | 0.97 | 0.85 | | | |
| StageI/II_vs_StageIII/IV | 0.7 (0.58-0.81) | | | | | | | | |
| bp | | 0.5625 | 0.76 (0.65-0.86) | 0.44 (0.24-0.64) | 0.81 | 0.37 | | | |
| fse | | 0.243 | 1.00 (1.00-1.00) | 0.04 (0.00-0.12) | 0.76 | 1 | | | |
| fsp | | 0.5805 | 0.76 (0.67-0.85) | 0.52 (0.32-0.72) | 0.83 | 0.41 | | | |

Note1: AUCs, Sensitivities and Specificities are reported as estimate (95% confidence interval)
Note2: PPV - Positive Predictive Value, NPV - Negative Predictive Value
Note 3: ′bp′, ′fse′ and ′fsp′ represent model-cutoffs taken at ″best point″ (closest point on ROC-curve to perfect classification) or with focus on (atleast 0.98) sensitivity (′fse′) or specificity (′fsp′).

1. An in vitro method of detecting, predicting or monitoring ovarian cancer in a subject, comprising determining in a sample from said subject the levels of the biomarkers in a panel comprising: TACSTD2, PROK1, MSMB, MUC-16, WFDC2, FR-alpha, and KRT19.

2. The method of claim 1, further comprising determining the levels of one or more of the following biomarkers: SPINT1, ICOSLG, CLEC6A, or CDH3.

3. The method of claim 1 or claim 2, wherein the levels of the following panels of biomarkers are determined:
(a) TACSTD2, PROK1, MSMB, MUC-16, WFDC2, FR-alpha, KRT19, and SPINT1; or
(b) TACSTD2, PROK1, MSMB, MUC-16, WFDC2, FR-alpha, KRT19, SPINT1 and ICOSLG; or
(c) TACSTD2, PROK1, MSMB, MUC-16, WFDC2, FR-alpha, KRT19, SPINT1, ICOSLG and CLEC6A; or
(d) TACSTD2, PROK1, MSMB, MUC-16, WFDC2, FR-alpha, KRT19, SPINT1, ICOSLG, CLEC6A and CDH3.

4. An in vitro method of detecting, predicting or monitoring ovarian cancer in a subject, comprising determining in a sample from said subject the level of two or more biomarkers selected from the list consisting of TACSTD2, PROK1, MSMB, MUC-16, WFDC2, FR-alpha, and KRT19, wherein at least one biomarker is selected from TACSTD2, PROK1 or MSMB.

5. The method of claim 4, wherein the biomarker PROK1 is selected.

6. The method of claim 4 or claim 5, wherein the biomarkers comprise at least TACSTD2, PROK1 and MSMB.

7. The method of any one of claims 4 to 6, wherein the levels of at least 3 or at least 4 or at least 5 biomarkers are determined.

8. The method of any one of claims 4 to 7, wherein the biomarkers comprise MUC-16, and optionally one or more of WFDC2, FR-alpha or KRT19.

9. The method of any one of claims 4 to 8, further comprising determining the levels of one or more of the following biomarkers: SPINT1, ICOSLG, CLEC6A, or CDH3.

10. The method of any one of claims 1 to 9, wherein the level of the biomarker is not determined in situ in an ovarian tissue sample.

11. The method of any one of claims 1 to 10, wherein based on the determined biomarker levels a biomarker profile for the subject is determined.

12. The method of claim 11, wherein the biomarker profile is compared to a control or reference biomarker profile.

13. The method of any one of claims 1 to 12, wherein:
(a) said method is a method of identifying a subject having ovarian cancer, wherein a change in the levels of the biomarkers, as compared with a control group of patients who do not have cancer, is indicative of ovarian cancer;
(b) said method is a method of identifying a subject at risk of developing ovarian cancer, wherein a change in the levels of the biomarkers, as compared with a control group of patients who do not have cancer, is indicative of a risk of developing ovarian cancer;
(c) said method is a method of assessing the efficacy of ovarian cancer treatment and comprises comparing the biomarker profiles in samples taken from a subject before and after the treatment or during the course of treatment, wherein a change in the biomarker profile over time toward a non-cancer profile or to a stable profile is interpreted as efficacy; or
(d) said method is a method of determining whether a subject potentially is developing cancer and comprises comparing the biomarker profiles in samples taken from a subject at two or more points in time, wherein a change in the biomarker profile toward a cancer profile, is interpreted as a progression toward developing cancer.

14. The method of any one of claims 1 to 13, wherein the sample is a sample from a subject who has, is suspected of having, or is at risk of having or developing ovarian cancer, optionally wherein the subject has symptoms of ovarian cancer.

15. The method of any one of claims 1 to 14, wherein the levels of biomarkers are determined by determining the levels of biomarker proteins, or of a protein fragment thereof.

16. The method of any one of claims 1 to 15, wherein the sample is a sample of body fluid or tissue other than ovarian tissue.

17. The method of any one of claims 1 to 15, wherein the sample is blood or a blood-derived sample.

18. The method of claim 16, wherein the sample is serum or plasma.

19. The method of any one of claims 1 to 17, wherein the levels of the biomarkers are determined using a proximity extension assay with proximity probes directed to detecting biomarker proteins.

20. The method of any one of claims 1 to 19, comprising determining whether an ovarian tumour in a subject is malignant or benign, and/or determining the stage of ovarian cancer based on the determined level of the biomarkers, and/or wherein the ovarian cancer is stage I or stage II ovarian cancer, or stage III or stage IV ovarian cancer.

21. Use of the combination of the proteins TACSTD2, PROK1, MSMB, MUC-16, WFDC2, FR-alpha, and KRT19 as biomarkers for ovarian cancer, optionally further including one or more of the biomarkers SPINT1, ICOSLG, CLEC6A, or CDH3.

22. A set of reagents to determine the levels of biomarkers in a sample, wherein the biomarkers comprise: TACSTD2, PROK1, MSMB, MUC-16, WFDC2, FR-alpha, and KRT19.

23. A set of reagents to determine the levels of biomarkers in a sample, wherein the biomarkers comprise two or more biomarkers selected from the list consisting of TACSTD2, PROK1, MSMB, MUC-16, WFDC2, FR-alpha, and KRT19, and wherein at least one biomarker is TACSTD2, PROK1 or MSMB.

24. The set of biomarkers of claim 22 or claim 23, wherein the biomarkers are as defined in any one of claims 2 to 3 or 5 to 9.

25. The set of reagents of any one of claims 21 to 24, wherein said reagents are binding agents capable of binding specifically to a said biomarker protein or to a fragment thereof.

26. The set of reagents of claim 25, wherein the binding agents are or comprise antibodies.

27. The set of reagents of claim 25 or claim 26, wherein the set of reagents comprises a pair of proximity probes for each biomarker protein, wherein each proximity probe comprises a binding domain and a nucleic acid domain conjugated thereto, and wherein each pair of proximity probes can bind simultaneously to their respective biomarker, or to a primary binding partner which is bound to the respective biomarker.
